(19) United States
(12) Patent Application Publication
(10) Pub. No.: US 2024/0360514 A1
(43) Pub. Date: Oct. 31, 2024

(54) METHODS OF PREDICTING LONG-TERM OUTCOME IN KIDNEY TRANSPLANT PATIENTS USING PRE-TRANSPLANTATION KIDNEY TRANSCRIPTOMES

(71) Applicant: University of Maryland, Baltimore, Baltimore, MD (US)

(72) Inventors: Valeria R. Mas, Ellicott City, MD (US); Daniel G. MALUF, Ellicott City, MD (US)

(73) Assignee: University of Maryland, Baltimore, Baltimore, MD (US)

(21) Appl. No.: 18/685,744

(22) PCT Filed: Aug. 30, 2022
(86) PCT No.: PCT/US2022/042026

§ 371 Date: Feb. 22, 2024

Related U.S. Application Data
(60) Provisional application No. 63/324,407, filed on Mar. 28, 2022.
(60) Provisional application No. 63/238,310, filed on Aug. 30, 2021.

(51) Int. Cl. C12Q 1/6883; C12Q 1/6806; C12Q 1/6851

(52) U.S. Cl. CPC C12Q 1/6883; C12Q 1/6851; C12Q 1/6806; C12Q 2600/118; C12Q 2600/158

(57) ABSTRACT

The first genome-wide, large-cohort study to demonstrate donor kidney transcriptomes can capture intrinsic organ quality and carry significant predictive weight for 24-month transplant function is disclosed. These findings shift the paradigm of understanding longer-term kidney transplant outcomes away from recipient factors/post-transplant events and towards intrinsic donor organ quality, which can be captured by molecular techniques. The combined predictive equation provided herein, using both clinical and biological data, can more accurately predict 24-month outcomes as compared to the current established scoring system (KDPI) in an external patient cohort.

### STATEMENT OF FEDERALLY SPONSORED RESEARCH AND DEVELOPMENT

This invention was made with government support under the Grant Numbers DK109581 and DK122682 awarded by the National Institutes of Health. The government has certain rights in the invention.

### TECHNICAL FIELD

The invention relates to methods for assaying transplant organ quality and predicting long-term transplant success.

### BACKGROUND OF INVENTION

Kidney transplantation (KT) significantly improves overall quality-of-life and survival for patients with end-stage renal disease (ESRD), however sustaining long-term allograft survival remains an ongoing challenge. [1] Also, a continuing shortage of donor organs has resulted in the increased use of marginal donor kidneys, complicating the development of objective markers for use in evaluating organ quality prior to transplantation. [2-4]

Currently, the evaluation of donor organ quality largely depends on the Kidney Donor Profile Index (KDPI), a numerical score that combines 10 donor characteristics with histological evaluations of core biopsies collected prior to transplantation. [5,6] The use of histology to predict short-term function was introduced nearly two decades ago when investigators reported that severe glomerulosclerosis increases the risk of delayed graft function (DGF) and poor 6-month outcomes. [7] However, histological scores at transplant time showed no correlation to long-term allograft survival. Histological evaluation has been widely disputed due to concerns related to bias and inter-observer discrepancies, yet this practice continues to be a standard of care in most US medical centers. [6] Thus far, clinical characteristics and histological findings have not allowed for a robust prediction of post-transplant function. [2,5-8]

Recent advances in transcriptomic technology have improved the diagnosis and management of human diseases. A transcriptomic profile serves as a snapshot of the temporary cell state and thus, its analysis can provide detailed and personalized information on the biological responses to injury. [9] Adapting transcriptome analysis for use in pre-transplantation analysis of donor organs may allow for the development of improved means for evaluation of donor organ quality. This would address the critical need for molecular tools that can accurately predict functional outcomes for kidney transplant patients and present a unique opportunity for molecular evaluations to assist in KT outcome prediction. [8]

The present invention is directed to these and other important goals.

### BRIEF SUMMARY OF INVENTION

With the development of novel prognostic tools derived from omics technologies, transplant medicine is entering the era of precision medicine, allowing surgeons to assay organs intended for transplant prior to transfer into a recipient. Such assaying can be used to determine the relative health of the organ as well as predict the probability that the organ will continue to function in the recipient for months or years once it has been transferred.

Currently, there are no established predictive biomarkers for post-transplant kidney function. The present invention addresses this deficiency. As further defined herein, the present invention is based on the results of a prospective multicenter study that led to the development and validation of a multivariable model, combining baseline clinical characteristics and transcriptomic (biological) data, that predicts posttransplant kidney function and that can be easily transferred to clinical settings. The prediction of long-term outcomes in patients receiving a kidney transplant has the potential to allow for early interventions to prevent or ameliorate progression to graft dysfunction, revealing a critical opportunity for transcriptomics to become a canon of contemporary transplant medicine.

In a first embodiment, the present invention is directed to a method of evaluating a kidney, comprising (a) obtaining a tissue sample from a kidney, (b) measuring expression levels of one or more predictive genes in said sample, (c) measuring expression levels of one or more housekeeping genes in said sample, and (d) calculating differences in expression levels measured for the one or more predictive genes versus expression levels measured for the one or more housekeeping genes, thereby evaluating a kidney.

In a second embodiment, the invention is directed to a method of evaluating functioning of a kidney, comprising (a) obtaining a tissue sample from a kidney, (b) measuring expression levels of one or more predictive genes in said sample, (c) measuring expression levels of one or more housekeeping genes in said sample, and (d) calculating differences in expression levels measured for the one or more predictive genes versus expression levels measured for the one or more housekeeping genes, thereby evaluating functioning of a kidney.

In certain aspects of this embodiment, the one or more predictive genes are associated with functional aspects of a kidney.

In a third embodiment, the invention is directed to a method of grading a kidney, comprising (a) obtaining a tissue sample from a kidney, (b) measuring expression levels of one or more predictive genes in said sample, (c) measuring expression levels of one or more housekeeping genes in said sample, (d) calculating differences in expression levels measured for the one or more predictive genes versus expression levels measured for the one or more housekeeping genes, and (e) comparing the differences calculated in (d) to standard values previously established for one or more other kidneys, thereby grading of a kidney.

In a fourth embodiment, the invention is directed to a method of determining a graft function risk score for a kidney, comprising (a) obtaining a tissue sample from a kidney, (b) measuring expression levels of one or more predictive genes in said sample, (c) measuring expression levels of one or more housekeeping genes in said sample, (d) calculating differences in expression levels measured for the one or more predictive genes versus expression levels measured for the one or more housekeeping genes, and (e) calculating a graft function risk score based on the differences calculated in (d), wherein the graft function risk score is calculated using the following formula (I)

$$graft function risk score = {b}_{0} + {b}_{1} ( {X}_{1} ) + {b}_{2} ( {X}_{2} ) + … {b}_{p} ( {X}_{p} ) \qquad ( I )$$

wherein b₀ is the intercept in the logistic regression model, wherein each b₁₋ₚ is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample.

In a specific aspect of this fourth embodiment, the invention is directed to a method of determining a graft function risk score for a kidney, comprising (a) obtaining a tissue sample from a kidney, (b) measuring expression levels of 13 predictive genes in said sample, (c) measuring expression levels of two housekeeping genes in said sample, (d) calculating differences in expression levels measured for each of the 13 predictive genes versus the mean value of expression levels measured for the two housekeeping genes, and (e) calculating a graft function risk score based on the differences calculated in (d), wherein the graft function risk score is calculated using the following formula (II)

$$graft function risk score = - 4.544 + 0.29 ( Δ Ct BCHE ) + 0.023 ( Δ Ct FKBP 4 ) - 0.981 ( Δ Ct GYPC ) - 0.105 ( Δ Ct HLA - DQB 1 ) - 0.327 ( Δ Ct HNRNPH 3 ) + 0.039 ( Δ Ct IGHD ) + 0.975 ( Δ Ct NUDT 4 ) + 0.717 ( Δ Ct RBM 8 A ) - 2.182 ( Δ Ct RHOQ ) + 0.112 ( Δ Ct SQLE ) + 1.073 ( Δ Ct STK 24 ) + 0.171 ( Δ Ct TRADD ) + 0.378 ( Δ Ct ZNFI 85 ) + 0.057 ( donor age ) + 0.004 ( donor BMI ) + 0.586 ( donor race indicator variable ) \qquad ( II )$$

wherein the donor race indicator variable=0 for Caucasian and 1 for all other races, wherein the 13 predictive genes are BCHE, FKBP4, GYPC, HLA-DQB1, IINRNPH3, IGHD, NUDT4, RBM8A, RHOQ, SQLE, SiK24, RADD, and ZNFT185, wherein the two housekeeping genes are ACTB and GAPDH, and wherein each ΔCt in formula (II) represents the calculated difference in expression level of indicated predictive genes versus the mean value of expression levels for the two housekeeping genes for each of the 13 predictive genes.

In a fifth embodiment, the invention is directed to a method of converting the graft function risk score discussed in the embodiments above into a probability score for a 0.0-1.0 probability scale, wherein the probability score is calculated using the following formula (III)

$$Probability score = \frac{{e}^{( {b}_{0} + {b}_{1} {X}_{1} + {b}_{2} {X}_{2} + ⋯ {b}_{p} {X}_{p} )}}{1 + {e}^{( {b}_{0} + {b}_{1} {X}_{1} + {b}_{2} {X}_{2} + ⋯ {b}_{p} {X}_{p} )}} \qquad ( III )$$

wherein b₀ is the intercept in the logistic regression model, wherein each b₁₋ₚ, is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample, and e=2.71828.

In non-limiting examples of the relevant embodiments of the invention as set forth herein, the predictive genes may be, but are not limited to, one or more of BCHE, FKBP4, GYPC, HLA-DQB1, HNRNPH3, IGHD, NUDT4, RBM8A, RHOQ, SQLE, STK24, TRADD, and ZNF185. In certain aspects, the predictive genes may be each BCHE, FKBP4, GYPC, HLA-DQB1, HNRNVPH3, IGHD, NUDT4, RBM8A, RHOQ, SQLE, STK24, TRADD, and ZNF185.

In non-limiting examples of the relevant embodiments of the invention as set forth herein, the housekeeping genes may be, but are not limited to, one or more of ACTB and GAPDH. In certain aspects, the housekeeping genes may be each of ACTB and GAPDH

In each of the relevant embodiments and aspects of the invention as set forth herein, the kidney may be a donor kidney.

In each of the relevant embodiments and aspects of the invention as set forth herein, the expression levels of the genes may be measured using qPCR.

In each of the relevant embodiments and aspects of the invention as set forth herein, the calculated differences in expression levels measured for the one or more predictive genes versus expression levels measured for the one or more housekeeping genes, is calculated using the mean value of expression levels measured for the housekeeping genes when the expression levels of two or more housekeeping genes are measured.

In each of the relevant embodiments and aspects of the invention as set forth herein, the graft function risk score may be one consideration in a decision of whether to transplant the kidney into a transplant recipient.

In each of the relevant embodiments and aspects of the invention as set forth herein, the graft function risk score may be used to predict whether the kidney will continue to function for at least 24 months in a transplant recipient receiving the kidney.

In aspects of the fifth embodiment, the probability score may be the probability that the kidney will continue to function for at least 24 months in a transplant recipient receiving the kidney.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Volcano plot showing fold changes and the adjusted p-values for all differentially expressed genes between groups at pre-transplantation (A.). The red dots represent down-regulated genes and blue dots represent up-regulated genes in low-functioning kidneys (B.) Heatmap of top enriched biological pathways in low-functioning kidneys, colored by p-values. Grey values indicate no detected expression patterns.

FIG. 2. Plot of the 55 genes listed by their variable importance in predicting 24-month function for the gene expression (GE) model (A.). Plot of the 52 variables (49 genes+3 donor characteristics) in order of variable importance used in predicting 24-month function for the gene expression+donor characteristics (G+D) model (B.).

FIG. 3. Area under the receiver operating characteristic (AUROC) curves for the training data for the donor characteristics (DC) model, gene expression (GE) model, gene expression+donor characteristics (G+D) model, and the KDPI model in predicting high vs. low eGFR group 24-months posttransplant. The diagonal line represents performance of a chance model.

FIG. 4. Area under the receiver operating characteristic (AUROC) curves for the validation set for the KDPI, donor characteristics (age, race, BMI), 14 genes alone, and 14 genes+3 donor characteristics in predicting high vs. low eGFR group 24-months post-transplantation. The diagonal line represents performance of a chance model.

FIG. 5. Probability score (derived from predictive equation) of each patient in the validation set (n=96) separated by 24-month outcome group (A.). Dotted horizontal line at 0.306 represents Youden's index. Mean and standard deviation bars displayed. Green represents high and red represents low 24-month function. KDPI score for each patient in the validation set separated by 24-month outcome group (B.). Dotted horizontal line at 52 represents Youden's index (where specificity and sensitivity are maximized). Mean and standard deviation bars displayed. KDPI and probability score of each patient plotted with Youden's indices depicted for each axis (C.).

FIG. 6. Patient flow diagram. A total of 295 patients were enrolled from 4 transplant centers (n=195 training set, n=100 validation set). Purple boxes represent exclusions. 21 patients were excluded from the training set due to follow-up loss, death with graft function, and microarray quality control criteria. 4 patients were excluded from the validation set due to low RNA integrity. The remaining 270 patients were included in the final training (n=174) and validation (n=96) sets. QC: Quality control; RIN: RNA integrity number.

FIG. 7. Spaghetti plot separated by high and low graft function group at 24 months with lowess smooths overlaid (A.). Smoothed eGFR post-transplant (black line) and fitted linear mixed effects model (white dotted line) with equation. Mean eGFR (corresponding to black line) and standard deviation at each timepoint separated by high and low 24-month graft function (B.).

FIG. 8. Kaplan-Meier estimates for time until graft failure or death showing graft/patient survival after 24-months, separated by 24-month graft function group with log-rank test comparing the two groups. Only patients who were alive at 24-months were included in the analyses, with 24-months as time-zero. NA: not available.

FIG. 9. Bar chart visualizing the top enriched cell-types for the upregulated DEGs (in low-functioning kidneys) and their associated q-values.

FIG. 10. Downregulated protein-protein interaction (PPI) networks encoded by differentially expressed genes (represented by nodes). Lines represent interaction relationships between nodes. Color-coded clusters were selected from PPI network using Molecular Complex Detection (MCODE). Downregulated pretransplant biological pathways associated with each MCODE subnetwork and adjusted p-values are listed.

FIG. 11. Upregulated protein-protein interaction (PPI) networks encoded by differentially expressed genes (represented by nodes). Lines represent interaction relationships between nodes. Color-coded clusters were selected from PPI network using Molecular Complex Detection (MCODE). Upregulated pretransplant biological pathways associated with each MCODE subnetwork and adjusted p-values are listed.

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

The field of transplantation is in critical need of more accurate tools to predict allograft outcomes. [19-21] Current in-use clinical scores and histological assessments have only demonstrated modest predictive accuracy for short-term outcomes. [22-25] Over the last decade, transcriptomic profiling has emerged as a powerful approach for revealing unbiased biological information useful for posttransplant management.

The study discussed herein represents the largest high-throughput transcriptomic analysis of pretransplant donor kidneys predicting 24-month outcomes conducted to date. The resulting data allowed development of the graft function risk score (GFRS) disclosed herein, which combines donor age, race, body mass index (BMI), and donor quality gene markers. The GFRS can be calculated prior to transplantation to predict graft function. The data also allowed the identification of differential pretransplant transcriptional profiles between kidneys with low and high function at 24-months, providing a deeper insight into the early biological processes leading to graft dysfunction.

The study was a prospective study having three critical features: i) inclusion of 270 patients from four transplant centers, ii) high-throughput genome-wide approaches, and iii) a well-characterized external validation cohort. Furthermore, the unique patient cohort included a broad spectrum of kidney donor organs (i.e., aged, DCD (donation after circulatory death), HCV+(hepatitis C virus), pumped, and AKI (acute kidney injury) donors), and a significant number of African American recipients (70.8%).

Thus far, a limited number of peer-reviewed pretransplant kidney gene expression studies have been conducted in the field. [26-34] Of these studies, only two evaluated graft outcomes beyond one year (both of which had small sample sizes and used targeted gene approaches). [30,34] Critically, none of the previous studies included external validations, which are necessary to determine the reproducibility and generalizability of results in different patient populations.

Additionally, the majority of predictive transcriptomic studies in kidney transplantation focused on delayed graft function (DGF) as a surrogate marker, without being able to predict longer-term outcomes (>12 months). [28,29,31-36] In the study reported herein, it was found that DGF was not significantly associated with 24-month function (p=0.238), explaining why gene sets associated with DGF have poor predictive value. [8,37] Furthermore, most transcriptomic studies have utilized post-reperfusion biopsies, which are less likely to capture intrinsic organ quality due to the ‘transcriptional noise’ induced by reperfusion injury, surgical procedures, recipient immune infiltration, and immunosuppressive medications. [8,28,30,38-40] The results presented herein indicate that the use of pre-reperfusion biopsies allows for a more accurate evaluation of donor organ quality. [8,41-43]

The results presented herein show that grafts with low function at 24 months displayed upregulated innate and adaptive immune responses (e.g., B cell proliferation, positive regulation of phagocytes, dendritic cell migration) prior to transplantation. This finding is in concordance with previous studies by the inventors, which reported an upregulated donor immune signature associated with short-term graft function. [²⁹,31,44] The inventors also recently reported that pretransplant donor biopsies from grafts progressing to chronic allograft dysfunction presented differentially methylated epigenetic profiles related to an activated immune state. [45]

Moreover, the downregulation of fundamental biological processes such as metabolic function (e.g., metabolism of cholesterol, carbon, and carbohydrates) further exacerbates the degree of injury posttransplant in kidneys with low 24-month function. Metabolic dysfunction in native kidney tissue (involving oxidative phosphorylation, fatty acid oxidation, cellular respiration) is associated with impaired repair mechanisms in kidney disease, [46-50] which may contribute to the progressive decline of graft function.

Overall, increased immune responses and decreased metabolic activity prior to transplantation disrupt graft homeostasis and result in the gradual loss of kidney function over time. These results are independent of cold ischemia time and other pre-/peri-transplant factors, reflecting the importance of evaluating the inherent donor mechanisms responsible for triggering and likely, sustaining post-transplant injury.

Although many genes have been identified to play important roles in kidney disease progression and pathophysiology, they do not inherently serve as reliable predictors of posttransplant graft function and disease state. [51] This study serves as one of the first computational studies to integrate experimental and clinical data to identify novel markers of graft function. All clinical and demographic characteristics from both the donors and recipients were analyzed, and statistically significant variables were used to develop a multivariable predictive model. As expected, donor age was the most predictive clinical variable, [8,29] followed by BMI and race. Current models including KDPI use less accessible/objective donor characteristics such as “history of hypertension” and “history of diabetes.” Interestingly, no recipient characteristics (including age, rejection events, or donor-specific antibodies) correlated with 24-month outcomes, demonstrating the prevailing importance of donor organ quality in predicting graft function.

As reported herein, 24-month graft function was more accurately predicted by the transcriptomic profile of preimplantation biopsies (GE model AUROC=0.994) than by significant donor characteristics (DC model AUROC=0.754) or by KDPI scores (KDPI model AUROC=0.718) (p<0.001). The same was true of the combined gene and donor characteristic (G+D) model (AUROC=0.996) (p<0.001).

To confirm the generalizability of these results, a small set of genes from the final models were tested in an independent cohort of patients (G-D model AUROC=0.821). This model more accurately predicted 24-month function than the KDPI (AUROC=0.691) and DC models (AUROC=0.691) (p=0.026). In the same patients, qPCR results and clinical characteristics were combined to develop a predictive equation quantifying patient risk for decreased 24-month graft function.

Defining surrogate endpoints, standards for outcome reporting, and statistical strategies to appropriately analyze differences between outcome groups is critical in biomarker discovery research. [52] Currently, there is a great deal of complexity associated with patient classification approaches in kidney transplantation. A reliable classification of kidney function and progression has been needed but prior to the present invention, it had not yet been achieved. Thus, when designing the study upon which the present invention is based, multiple different patient classification approaches were considered that utilized one or more of the following parameters: overall eGFR slope, Y-intercepts, final eGFR as a continuous outcome, and multiple eGFR measurements. Analysis of estimated glomerular filtration rate (eGFR) was selected as a dichotomous outcome to enable the reporting of clinically meaningful statistics that frequently accompany diagnostic/prognostic assays, such as the AUROC. Ultimately, this eGFR categorization (supported by significant differences in long-term graft survival) allows for significant statistical power to detect important differences across primary endpoints for direct clinical translation. [52,53]

The present invention thus discloses the first genome-wide large-cohort study to demonstrate that the donor kidney transcriptome, prior to implantation, captures intrinsic organ quality and carries significant predictive weight for 24-month transplant function. The findings presented herein shift the paradigm of understanding longer-term kidney transplant outcomes away from recipient factors/post-transplant events (e.g., DGF) and towards the intrinsic donor organ quality, which can be captured by molecular techniques. Notably, the invention demonstrates that a combined predictive equation using both clinical and biological data can more accurately predict 24-month outcomes as compared to the current established scoring system (KDPI) in an external patient cohort.

In more detail, the study that underpins the present invention included a total of 270 deceased donor pretransplant kidneys from which biopsies were collected and for which posttransplant function was prospectively monitored. In the study, the utility of pretransplant gene expression profiles in predicting 24-month outcomes was first assessed in a training set (n=174). Nearly 600 differentially expressed genes were associated with 24-month graft function. Grafts that progressed to low function at 24-months exhibited upregulated immune responses and downregulated metabolic processes at pretransplantation. Using penalized logistic regression modeling, a 55 gene model AUROC for 24-month graft function was 0.994. Gene expression for a subset of candidate genes was then measured in an independent set of pretransplant biopsies (n=96) using qPCR. The AUROC when using 13 genes with 3 donor characteristics (age, race, BMI) was 0.821. Subsequently, a graft function risk score was calculated using this combination for each patient in the validation cohort, demonstrating the translational feasibility of using gene markers as prognostic tools. The graft function risk score can also be converted into a probability score for a 0.0-1.0 probability scale, based on the probability of low 24-month graft function. These findings support the potential of pretransplant transcriptomic biomarkers as novel instruments for improving posttransplant outcome predictions and associated management.

The results from the study disclosed in the Examples below provide the basis for the present invention. The results have allowed the inventors produce different methods for assaying kidneys, grading or scoring kidneys, and making predictions about both short- and long-term functioning of transplanted kidneys. These different methods have the same underlying basis in the results presented herein and thus form closely related subject matter. The methods can be described in the context of the five different embodiments discussed in the following paragraphs.

In a first embodiment, the present invention is directed to a method of evaluating a kidney, comprising (a) obtaining a tissue sample from a kidney, (b) measuring expression levels of one or more predictive genes in said sample, (c) measuring expression levels of one or more housekeeping genes in said sample, and (d) calculating differences in expression levels measured for the one or more predictive genes versus expression levels measured for the one or more housekeeping genes, thereby evaluating a kidney.

When the expression levels of two or more housekeeping genes are measured, the differences in expression levels are calculated using the mean value of expression levels measured for the housekeeping genes.

In a second embodiment, the invention is directed to a method of evaluating functioning of a kidney, comprising (a) obtaining a tissue sample from a kidney, (b) measuring expression levels of one or more predictive genes in said sample, (c) measuring expression levels of one or more housekeeping genes in said sample, and (d) calculating differences in expression levels measured for the one or more predictive genes versus expression levels measured for the one or more housekeeping genes, thereby evaluating functioning of a kidney.

In certain aspects of this embodiment, the one or more predictive genes are associated with functional aspects of a kidney. Functional aspects of a kidney include, but are not limited to, metabolic functions, immune activation and apoptosis.

When the expression levels of two or more housekeeping genes are measured, the differences in expression levels are calculated using the mean value of expression levels measured for the housekeeping genes.

In a third embodiment, the invention is directed to a method of grading a kidney, comprising (a) obtaining a tissue sample from a kidney, (b) measuring expression levels of one or more predictive genes in said sample, (c) measuring expression levels of one or more housekeeping genes in said sample, (d) calculating differences in expression levels measured for the one or more predictive genes versus expression levels measured for the one or more housekeeping genes, and (e) comparing the differences calculated in (d) to standard values previously established for one or more other kidneys, thereby grading of a kidney.

It should be understood that the “grading” can be made in different or multiple formats. For example, the grading can be on a numeric scale, such as 1 to 3, 1 to 5, and 1 to 10, or on a letter-based based scale, such as A-C. However, the grading with generally be based on whether and what level the kidney being graded is expected to be functional in the recipient, either in the short-term, long-term, or both. Functional means that the kidney will maintain normal functions associated with a kidney, although the level of functionality may be the same or less, compared to the function of a kidney that has not been transplanted.

When the expression levels of two or more housekeeping genes are measured, the differences in expression levels are calculated using the mean value of expression levels measured for the housekeeping genes.

In a fourth embodiment, the invention is directed to a method of determining a graft function risk score for a kidney, comprising (a) obtaining a tissue sample from a kidney, (b) measuring expression levels of one or more predictive genes in said sample, (c) measuring expression levels of one or more housekeeping genes in said sample, (d) calculating differences in expression levels measured for the one or more predictive genes versus expression levels measured for the one or more housekeeping genes, and (e) calculating a graft function risk score based on the differences calculated in (d), wherein the graft function risk score is calculated using the following formula (I)

$$graft function risk score = {b}_{0} + {b}_{1} ( {X}_{1} ) + {b}_{2} ( {X}_{2} ) + … {b}_{p} ( {X}_{p} ) \qquad ( I )$$

wherein b₀ is the intercept in the logistic regression model, wherein each b₁₋ₚ is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample.

In a specific aspect of this fourth embodiment, the invention is directed to a method of determining a graft function risk score for a kidney, comprising (a) obtaining a tissue sample from a kidney, (b) measuring expression levels of 13 predictive genes in said sample, (c) measuring expression levels of two housekeeping genes in said sample, (d) calculating differences in expression levels measured for each of the 13 predictive genes versus the mean value of expression levels measured for the two housekeeping genes, and (e) calculating a graft function risk score based on the differences calculated in (d), wherein the graft function risk score is calculated using the following formula (II)

$$graft function risk score = - 4.544 + 0.29 ( Δ Ct BCHE ) + 0.023 ( Δ Ct FKBP 4 ) - 0.981 ( Δ Ct GYPC ) - 0.105 ( Δ Ct HLA - DQB 1 ) - 0.327 ( Δ Ct HNRNPH 3 ) + 0.039 ( Δ Ct IGHD ) + 0.975 ( Δ Ct NUDT 4 ) + 0.717 ( Δ Ct RBM 8 A ) - 2.182 ( Δ Ct RHOQ ) + 0.112 ( Δ Ct SQLE ) + 1.073 ( Δ Ct STK 24 ) + 0.171 ( Δ Ct TRADD ) + 0.378 ( Δ Ct ZNFI 85 ) + 0.057 ( donor age ) + 0.004 ( donor BMI ) + 0.586 ( donor race indicator variable ) \qquad ( II )$$

wherein the donor race indicator variable=0 for Caucasian and 1 for all other races, wherein the 13 predictive genes are BCHE, FKBP4, GYPC, HLA-DQB1, HNRNPH3, IGHD, NUDT4, RBM8A, RHOQ, SQLE, STK24, TRADD, and ZNF185, wherein the two housekeeping genes are ACTB and GAPDH, and wherein each ΔCt in formula (II) represents the calculated difference in expression level of indicated predictive genes versus the mean value of expression levels for the two housekeeping genes for each of the 13 predictive genes.

In a fifth embodiment, the invention is directed to a method of converting the graft function risk score discussed in the embodiments above into a probability score for a 0.0-1.0 probability scale wherein the probability score is calculated using the following formula (III)

$$Probability score = \frac{{e}^{( {b}_{0} + {b}_{1} {X}_{1} + {b}_{2} {X}_{2} + ⋯ {b}_{p} {X}_{p} )}}{1 + {e}^{( {b}_{0} + {b}_{1} {X}_{1} + {b}_{2} {X}_{2} + ⋯ {b}_{p} {X}_{p} )}} \qquad ( III )$$

wherein b₀ is the intercept in the logistic regression model, wherein each b₁₋ₚ is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample, and e=2.71828.

In each of the relevant embodiments and aspects of the invention as set forth herein, the kidney may be a donor kidney.

In each embodiment and aspect of the invention, the subject is any vertebrate animal having a kidney including, but not limited to, human, non-human primate, bird, horse, cow, goat, sheep, a companion animal, such as a dog, cat or rodent, or other mammal having a kidney. By the sake token, the kidney may be the kidney of any vertebrate animal having a kidney including, but not limited to, human, non-human primate, bird, horse, cow, goat, sheep, a companion animal, such as a dog, cat or rodent, or other mammal having a kidney.

In each of the relevant embodiments and aspects of the invention as set forth herein, the calculated differences in expression levels measured for the one or more predictive genes versus expression levels measured for the one or more housekeeping genes, is calculated using the mean value of expression levels measured for the housekeeping genes when the expression levels of two or more housekeeping genes are measured.

In each of the relevant embodiments and aspects of the invention as set forth herein, the graft function risk score may be one consideration in a decision of whether the transplanted kidney will have a higher risk of graft dysfunction at 24-months posttransplant. Other considerations that may be used include, but are not limited to, whether to transplant the kidney into a transplant recipient

In each of the relevant embodiments and aspects of the invention as set forth herein, the graft function risk score may be used to predict whether the kidney will continue to function for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more months in a transplant recipient receiving the kidney. In a particular aspect of the invention, the graft function risk score may be used to predict whether the kidney will continue to function for at least 24 months in a transplant recipient receiving the kidney.

In aspects of the fifth embodiment, the probability score may be the probability that the kidney will continue to function for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more months in a transplant recipient receiving the kidney. In a particular aspect of the invention, the probability score may be the probability that the kidney will continue to function for at least 24 months in a transplant recipient receiving the kidney.

### Predictive Genes

In each of the embodiments and aspects of the invention, the predictive genes may be, but are not limited to, one or more of:
BCHE (butyrylcholinesterase),
FKBP4 (FKBP Prolyl Isomerase 4),
GYPC (Glycophorin C),
HLA-DQB1 (Major Histocompatibility Complex, Class II, DQ Beta 1),
HNRNPH3 (Heterogeneous Nuclear Ribonucleoprotein H3),
IGHD (Immunoglobulin Heavy Constant Delta),
NUDT4 (Nudix Hydrolase 4),
RBM8A (RNA Binding Motif Protein 8A),
RHOQ (Ras Homolog Family Member Q),
SQLE (Squalene Epoxidase),
S7K24 (Serine/Threonine Kinase 24),
TRADD (Tumor necrosis factor receptor type 1-associated DEATH domain), and
ZNF185 (Zinc Finger Protein 185 With LIM Domain).

In addition, the predictive genes may be one or more of the genes provided in Table 2, one or more of the genes provided in Table 4, or one or more of the genes provided in Table 9. Although the 13 genes listed above were selected for validation, a total of 53 genes were identified as part of the donor gene (GE) model shown in Table 2, and 49 genes were identified as part of the donor (G+D) model shown in Table 4. Moreover, the list of differentially expressed genes associated with 24-months outcomes also presents diagnostic potential (Table 9), where 595 unique genes (corresponding to 699 probe sets) were differentially expressed (FDR<0.05) in pretransplant donor organs, of which 408 were upregulated and 187 were downregulated in low function kidneys).

In certain aspects, the predictive genes may be each BCHE, FKBP4, GYPC, HLA-DQB1, HNRNPH3, IGHD, NUDT4, RBM8A, RHOQ, SQLE, STK24, TRADD, and ZNF185.

### Housekeeping Genes

In each of the embodiments and aspects of the invention, the housekeeping genes may be, but are not limited to, one or more of:
ACTB (Actin Beta), and
GAPDH (glyceraldehyde-3-phosphate dehydrogenase).

In certain aspects, the housekeeping genes may be each of ACTB and GAPDH.

### Means for Obtaining Kidney Tissue Sample

It will be understood that a tissue sample may be obtained from a kidney using any art-recognized method for obtaining a tissue sample without causing undue injury to the kidney. As a non-limiting example, a tissue sample may be obtained using an 18-gauge biopsy needle. The sample may be further processed by immediately suspended it in a protective solution, such as RNAlater (Ambion, Austin, USA). The sample may be obtained before or after it is removed from the donor.

### Means for Measuring Expression Levels

In each of the relevant embodiments and aspects of the invention as set forth herein, the expression levels of the predictive and housekeeping genes may be measured using qPCR (quantitative polymerase chain reaction or real time polymerase chain reaction).

### III. Examples

The following paragraphs provide the materials and methods that were used in the experiments.

Patients and Samples. A total of 295 consecutive deceased donor (DD) kidney transplant (KT) recipients were enrolled from four transplant centers in the US, including 1) Virginia Commonwealth University (VCU) Medical Center, 2) University of Virginia (UVA) Medical Center, 3) Montefiore Medical Center, and 4) University of Tennessee Health Science Center (UTHSC). The study protocol was approved by the Institutional Review Board (IRB #1HP-00092097). The clinical and research activities being reported are consistent with the Principles of the Declaration of Istanbul as outlined in the Declaration of Istanbul on Organ Trafficking and Transplant Tourism. Written informed consent was obtained from KT recipients at transplantation time. Living donor recipients, retransplant recipients, pregnant women, recipients <18 years old, HIV+recipients, and recipients with previous history of malignancy were excluded from the study.

Tissue was obtained shortly before transplantation (back-bench biopsies) using an 18-gauge biopsy needle and immediately suspended in RNAlater (Ambion, Austin, USA). Patients received triple immunosuppression with calcineurin inhibitors, mycophenolate mofetil, and steroids. For induction therapies, either anti-thymocyte globulin or basiliximab were administered.

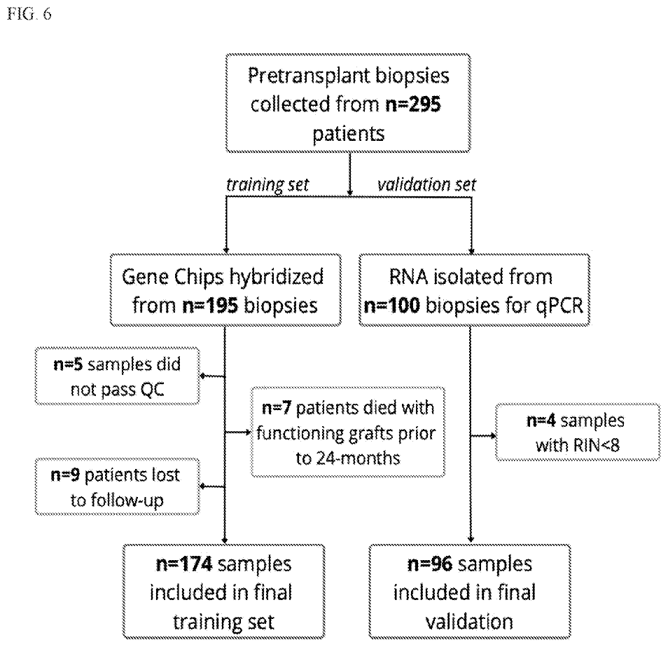

Samples collected from UVA and VCU were included as part of the training set, while samples collected from Montefiore and UTHSC were included as part of the external validation set. Out of the 295 patients enrolled, a total of 25 were excluded due to follow-up loss, death with graft function, microarray quality control criteria, and biopsy RNA integrity. The patient flow diagram is shown in FIG. 6.

Pre-processing Methods. Total RNA was isolated from renal biopsies using TRIzol reagent (Invitrogen, Waltham, USA). RNA quality and integrity were evaluated using the Agilent RNA 6000 Nano Kit on the Agilent 2100 Bioanalyzer (Agilent Technologies, Santa Clara, USA). Samples with an RNA integrity number of <8 were excluded from the analysis.

Gene expression of biopsies from the training set was measured using Affymetrix GeneChip microarrays (HG-U133A 2.0) (access: GSE147451) (Thermo Fisher Scientific, Waltham, USA). The Affymetrix Detection Call algorithm was used to determine whether probe sets were present, marginally present, or absent in each sample. Quality control was performed as previously published. [10] To obtain probe set expression summaries, the robust multiarray average method was used. [11] Prior to statistical analysis, the gene expression data matrix was filtered to exclude probe sets called absent in all samples and control probe sets, leaving 19,380 probe sets remaining for statistical analysis.

Study Design. Estimated Glomerular Filtration Rate (eGFR) was calculated using the abbreviated Modification of Diet in Renal Disease (MDRD) formula. [12] Study endpoints were defined as graft function at 24-months post-transplant (mean=24.3±1.2 months). Categorically, patients were considered to have low graft function with a 24-month eGFR<45 mL/min/1.73 m², while an eGFR of ≥45 mL/min/1.73 m² represented the high function group, corresponding to the chronic kidney disease KDIGO guidelines (www.kidney-international.org). Additionally, patients who experienced graft failure prior to 24-months were included in the low-functioning group. Linear mixed-effects models that included eGFR recorded at all time points (1-, 6-, 9-, 12-, 18-, 21-, and 24-months post-KT) were fit to demonstrate how continuous eGFR differed by this dichotomous categorization. To assess long-term outcomes, graft/patient survival was calculated as the time from 24-month post-transplant until the date of graft failure or date of death, censoring for those alive without graft failure at their last follow-up date. Only patients alive at 24-months were included in the survival analysis.

Statistical Methods. The Kaplan-Meier method was used to estimate graft/patient survival and the log-rank test was used to compare the high vs. low eGFR groups. Descriptive statistics (mean and standard deviation (SD)) were applied to summarize continuous variables, while frequencies and percentages were used to summarize categorical variables.

To identify differentially-expressed genes (DEGs) associated with outcome group, probe set level linear models were fit with high vs. low graft function group assignments as the predictor variable adjusting for the surrogate variable representing batch effect, using the limma Bioconductor package of the open-source R software for statistical computing and graphics (R Foundation for Statistical Computing, Vienna, Austria). All resulting p-values were adjusted for multiple hypothesis testing using Benjamini and Hochberg's false discovery rate (FDR) method. [13]

Penalized logistic regression models were applied to simultaneously perform automatic variable selection and outcome prediction for high-dimensional covariate spaces. First, the gene expression data matrix was filtered to retain differentially expressed probe sets having an FDR<0.05. Thereafter, repeated 10-fold cross-validation (CV) was used to identify the optimal tuning parameters for fitting a penalized logistic regression model predicting outcome (high vs. low graft function). The repeated 10-fold CV procedure was performed using the caret package [14] with glmnet [15] in the R programming environment. Gene expression data was applied to derive a multivariable model. A grid search was performed to optimize the two tuning parameters required by elastic net, the penalty term λ, and the proportion of the penalty associated with the LASSO versus ridge regression, αLASSO. The combination of DEGs that optimized the area under the receiver operating characteristic curve (AUROC) from the repeated 10-fold CV procedure was selected for fitting the gene expression model. Significant demographic/clinical characteristics (p-value<0.05) were combined with DEGs to develop a gene expression+clinical data model. Two additional models were fit for performance comparison: one using all significant clinical characteristics and another that included the patient's numerical KDPI value as the sole predictor. [16]

Pathway Analyses. GO and KEGG pathway enrichment analyses were performed using enrichGO and enrichKEGG functions which adjust the estimated significance level to account for multiple hypothesis testing (FDR≤0.05). Finally, Metascape (metascape.org) was used for functional enrichment, interactome analysis, gene annotation, cell enrichment, and protein-protein interactions (PPIs). [17] The Molecular Complex Detection (MCODE) algorithm was applied to the PPI network to identify densely connected networks.

QPCR Validation. An initial set of genes was selected for further validation based on i) statistical significance, ii) high predictive performance in final models, and iii) association with relevant biological pathways. Individual predesigned TaqMan™ assays (ThermoFisher Scientific, Waltham, USA) were used for qPCR reactions. Gene expression results were expressed as ΔCt values normalized by a dual reference gene combination (ACTB and GAPDH). [18] Univariable logistic regression models were fit for each gene to identify whether gene expression was significantly associated with 24-month outcome. Thereafter, multivariable logistic regression models were fit for each gene to determine significance after adjusting for important clinical covariates identified in the training set, and the AUROC and associated 95% confidence intervals (CI) were estimated.

Risk Score Equation. The estimated regression coefficients (b) for each independent variable (A) in the multivariable regression model were used to form the linear graft function risk score equation shown in formula (I):

$$graft function risk score = {b}_{0} + {b}_{1} ( {X}_{1} ) + {b}_{2} ( {X}_{2} ) + … {b}_{p} ( {X}_{p} ) \qquad ( I )$$

The optimal threshold which maximizes both specificity and sensitivity (Youden's index) was used to predict whether the subjects would have low or high eGFR at 24 months. Lastly, the linear predictor (risk score) for each patient was converted into a probability score (0.0-1.0) using the equation shown in formula (III):

$$Probability score = \frac{{e}^{( {b}_{0} + {b}_{1} {X}_{1} + {b}_{2} {X}_{2} + ⋯ {b}_{p} {X}_{p} )}}{1 + {e}^{( {b}_{0} + {b}_{1} {X}_{1} + {b}_{2} {X}_{2} + ⋯ {b}_{p} {X}_{p} )}} \qquad ( III )$$

The following paragraphs provide the results from the experiments.

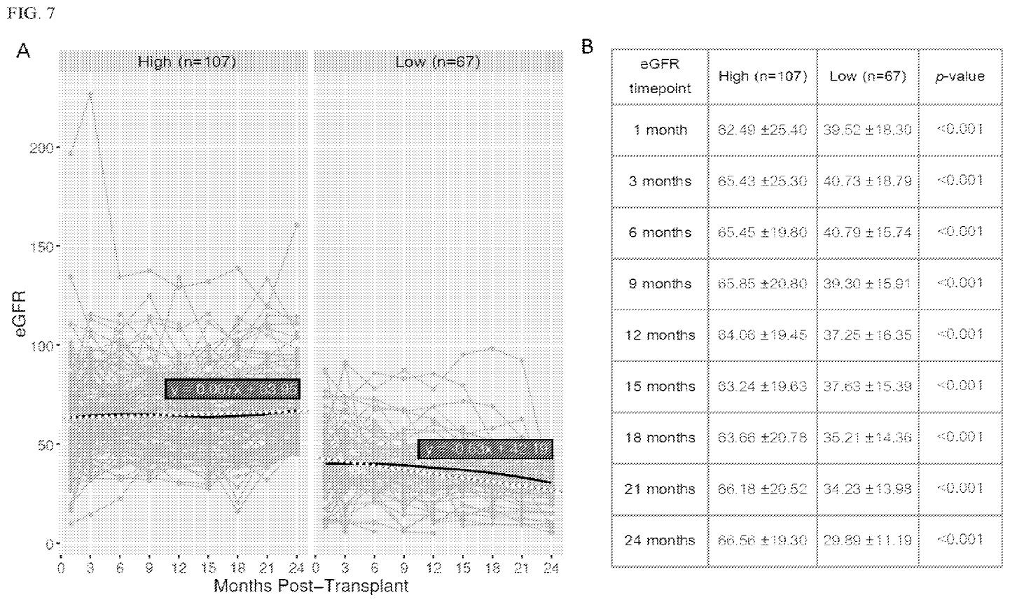

Clinical markers discriminating 24-month eGFR outcomes. Among the 174 KT recipients in the training set, 67 (38.5%) subjects had low graft function and 107 (61.5%) had high function based on the criteria described above. Clinical characteristics and demographics are shown in Table 1. On average, the high functioning group was composed of younger donor kidneys (37±16 years) compared to the low graft function group (48±14 years) (p<0.001). The groups also differed with respect to donor race (p=0.006), and BMI (p<0.001). No recipient variables were significantly different between groups. A spaghetti plot separated by high vs. low graft function with lowess smooths overlaid and the linear mixed-effects model demonstrated the difference between the eGFR trajectories over time (FIG. 7). Regarding the individual eGFR courses, there was a significant difference (p<0.001) between the two groups across each timepoint throughout the 24-month period of observation. The high-functioning group showed a stable positive eGFR slope of 0.067 ml/min/month (0.81 ml/min/year), while the low-functioning group had a negative slope of −0.53 ml/min/month (−6.36 ml/min/year).

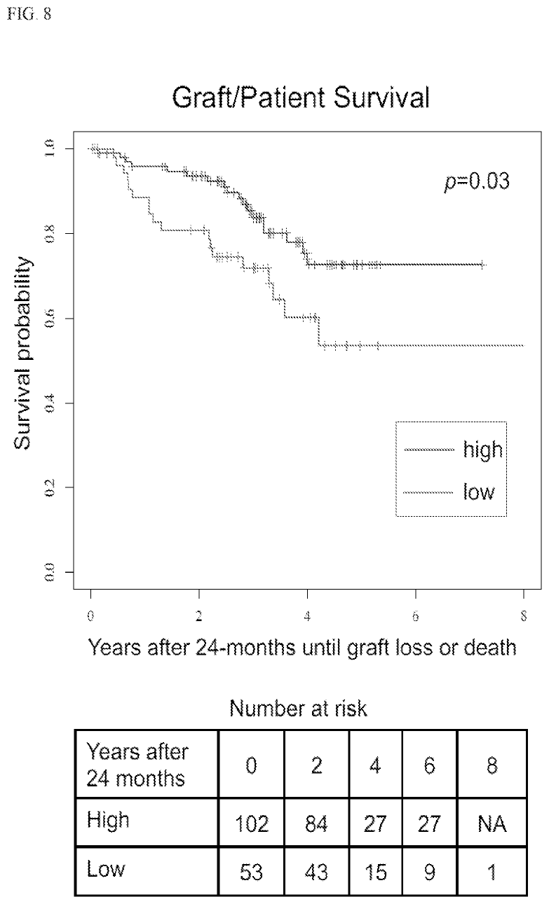

Patients with low 24-month graft function experienced significantly poorer long-term survival outcomes than patients with high 24-month graft function (p=0.03) (FIG. 8). Using the combined analytical approaches, it was evident that the two groups were significantly different throughout follow-up.

TABLE 1
Characteristics of donor and recipients sub-stratified based on eGFR at 24-month
post kidney transplant in the training set (n = 174). A two-sample t-test
was computed for continuous variables, while categorical variables were compared
using a Chi-square test (or Fisher's exact test when there were small cell sizes).

| | | | | |
|---|---|---|---|---|
| | | High eGFR | Low eGFR | |
| Clinical Characteristic | Category | (n = 107) | (n = 67) | p-value |

Donor Characteristics

| Donor age, years | | 37.12 ± 15.97 | | 48.49 ± 13.79 | | <0.001 |
|---|---|---|---|---|---|---|
| (avg ± SD) | | | | | | |
| Donor gender | Male | 66 | (61.7) | 36 | (53.7) | 0.38 |
| n (%) | Female | 41 | (38.3) | 31 | (46.3) | |
| Donor race | American Indian | 1 | (0.9) | 0 | (0.0) | 0.006♦ |
| n (%) | Asian | 2 | (1.9) | 0 | (0.0) | |
| | African American | 24 | (22.4) | 30 | (44.8) | |
| | Caucasian | 79 | (73.8) | 37 | (55.2) | |
| | Hispanic | 1 | (0.9) | 0 | (0.0) | |
| DCD, n (%) | | 16 | (15.0) | 12 | (17.9) | 0.761 |
| Donor cause of death | Anoxia | 33 | (30.8) | 20 | (29.9) | 0.113♦ |
| n (%) | Head trauma | 36 | (33.6) | 13 | (19.4) | |
| | Stroke | 34 | (31.8) | 32 | (47.8) | |
| | Other/Unknown | 4 | (3.7) | 2 | (3.0) | |
| Delayed graft function | | 34 | (31.8) | 28 | (41.8) | 0.238 |
| n (%) | | | | | | |
| Donor BMI | | 26.57 ± 5.83 | | 31.10 ± 9.07 | | <0.001 |
| (avg ± SD) | | | | | | |
| CIT, hours (avg ± SD) | | 19.48 ± 9.01 | | 19.73 ± 6.65 | | 0.837 |
| WIT, min (avg ± SD) | | 30.79 ± 7.33 | | 31.79 ± 6.82 | | 0.367 |
| Pump used, n (%) | | 53 | (49.5) | 44 | (65.7) | 0.054 |
| Pump time | | 7.05 ± 8.06 | | 7.84 ± 7.16 | | 0.497 |
| hours (avg ± SD) | | | | | | |
| Last donor creatinine | | 1.24 ± 0.87 | | 1.25 ± 0.55 | | 0.903 |
| mg/dL (avg ± SD) | | | | | | |
| Donor HBV cAb | Negative | 93 | (86.9) | 61 | (91.0) | 0.613♦ |
| n (%) | Positive | 9 | (8.4) | 3 | (4.5) | |
| | N/A | 5 | (4.7) | 3 | (4.5) | |
| Donor HCV Ab | Positive | 12 | (11.2) | 7 | (10.4) | 1.00 |
| n (%) | | | | | | |
| Donor CMV, n (%) | Positive | 63 | (58.9) | 42 | (62.7) | 0.734 |

| | | | |
|---|---|---|---|
| KDPI (avg ± SD) | 49.46 ± 27.40 | 69.93 ± 22.00 | <0.001 |
| KDRI (avg ± SD) | 1.07 ± 0.36 | 1.34 ± 0.40 | <0.001 |

Histological Evaluation of Pretransplant Biopsies

| | | | | | | |
|---|---|---|---|---|---|---|
| Pretransplant | Absent | 62 | (57.9) | 46 | (68.6) | 0.603♦ |
| glomerulosclerosis | Mild | 17 | (15.9) | 8 | (11.9) | |
| (gsc) | Moderate | 2 | (1.9) | 1 | (1.5) | |
| n (%) | Severe | 0 | (0.0) | 0 | (0.0) | |
| | N/A | 26 | (24.3) | 12 | (17.9) | |
| Pretransplant | Absent | 25 | (23.4) | 10 | (14.9) | 0.160♦ |
| interstitial fibrosis (if) | Mild | 52 | (48.6) | 39 | (58.2) | |
| n (%) | Moderate | 4 | (3.7) | 6 | (9.0) | |
| | Severe | 0 | (0.0) | 0 | (0.0) | |
| | N/A | 26 | (24.3) | 12 | (17.9) | |
| Pretransplant tubular | Absent | 46 | (43.0) | 26 | (38.8) | 0.263♦ |
| atrophy (ta) | Mild | 34 | (31.8) | 26 | (38.8) | |
| n (%) | Moderate | 1 | (0.9) | 3 | (4.5) | |
| | Severe | 0 | (0.0) | 0 | (0.0) | |
| | N/A | 26 | (24.3) | 12 | (17.9) | |

Recipient Characteristics

| Recipient age | | 51.98 ± 12.62 | | 53.09 ± 11.06 | | 0.556 |
|---|---|---|---|---|---|---|
| (avg ± SD) | | | | | | |
| Recipient gender | Male | 64 | (59.8) | 40 | (59.7) | 1.00 |
| n (%) | Female | 43 | (40.2) | 27 | (40.3) | |
| Recipient race | Asian/Pacific | 1 | (0.9) | 0 | (0.0) | 0.898♦ |
| n (%) | Islander | | | | | |
| | African American | 79 | (73.8) | 50 | (74.6) | |
| | Caucasian | 22 | (20.6) | 16 | (23.9) | |
| | Hispanic | 4 | (3.7) | 1 | (1.5) | |
| | Other/Unknown | 1 | (0.9) | 0 | (0.0) | |
| Recipient BMI, | | 27.92 ± 5.19 | | 28.48 ± 4.86 | | 0.479 |
| (avg ± SD) | | | | | | |
| Recipient HCV, | Positive | 13 | (12.1) | 6 | (9.0) | 0.621♦ |
| n (%) | Negative | 94 | (87.9) | 61 | (91.0) | |
| CMV disease, n (%) | Positive | 2 | (1.9) | 4 | (6.0) | 0.206♦ |
| Recipient CMV | Positive | 82 | (76.6) | 51 | (76.1) | 1.00 |
| n (%) | | | | | | |
| Pretransplant diagnosis | DM | 20 | (18.7) | 15 | (22.4) | 0.516♦ |
| n (%) | DM/HTN | 24 | (22.4) | 8 | (11.9) | |
| | HTN | 37 | (34.6) | 25 | (37.3) | |
| | FSGS | 8 | (7.5) | 5 | (7.5) | |
| | Other | 18 | (16.8) | 14 | (20.9) | |
| Matched sex, n (%) | | 49 | (45.8) | 41 | (61.2) | 0.068 |
| Months on dialysis | | 40.37 ± 34.62 | | 45.95 ± 37.51 | | 0.333 |
| pretransplant | | | | | | |
| (avg ± SD) | | | | | | |
| AR episodes within 12 | | 10 | (9.3) | 10 | (14.9) | 0.330♦ |
| months posttransplant | | | | | | |
| n (%) | | | | | | |
| HLA mismatch | | 4.38 ± 1.33 | | 4.41 ± 1.21 | | 0.768 |
| (avg ± SD) | | | | | | |
| PRA | >80% | 30 | (28.0) | 22 | (32.8) | 0.501 |
| dnDSA, | Positive | 8 | (7.5) | 10 | (14.9) | 0.131♦ |
| n (%) | | | | | | |

♦Fisher's exact test used due to small expected cell sizes.
AR: acute rejection; BMI: body mass index; CIT: cold ischemia time; CMV: cytomegalovirus; DCD: donation after circulatory death; DM: diabetes mellitus; dnDSA: de novo donor specific antibody, FSGS: focal segmental glomerulosclerosis; HBV: hepatitis B virus; HCV: Hepatitis C virus; HLA: human leukocyte antigen; HTN: hypertension; KDPI: Kidney Donor Profile Index; KDRI: Kidney Donor Risk Index; SCD: standard criteria donor; WIT: warm ischemia time.

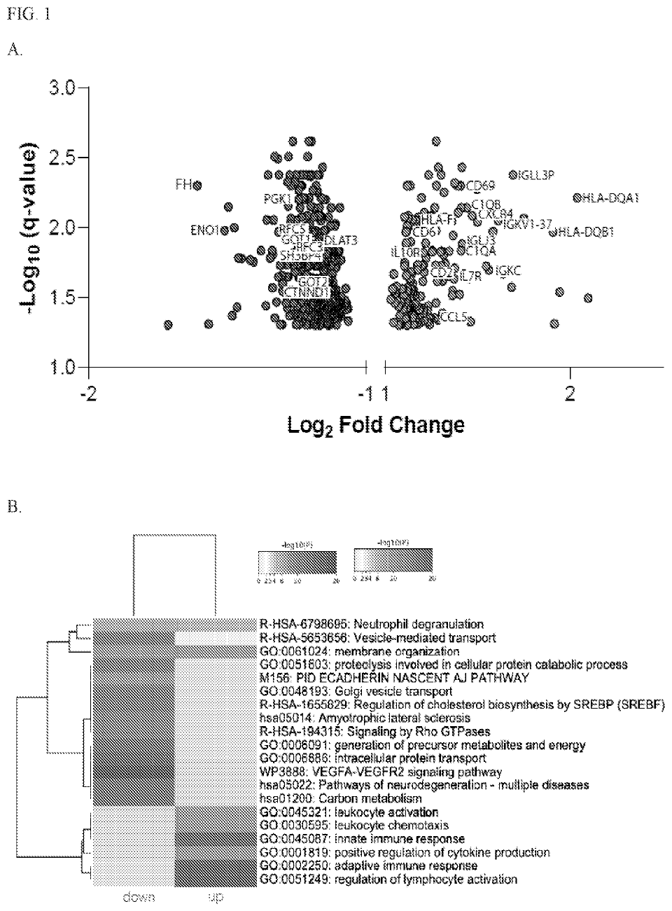

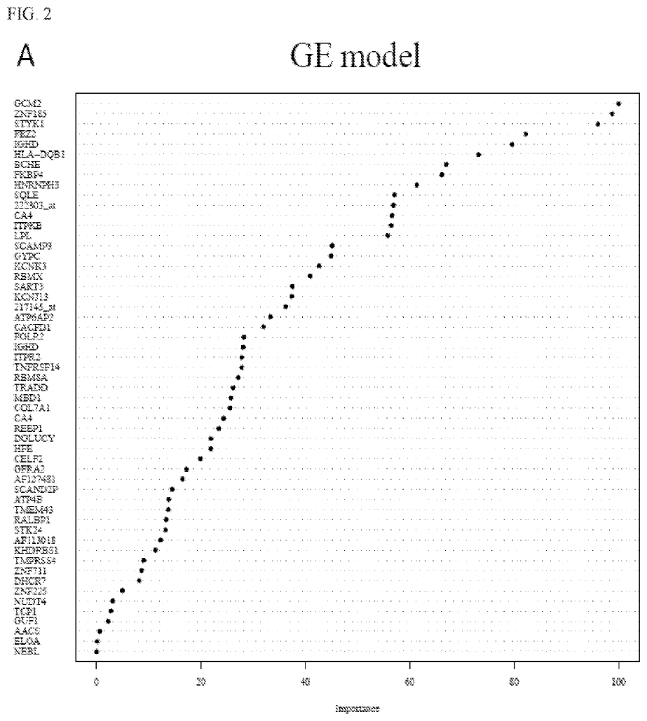

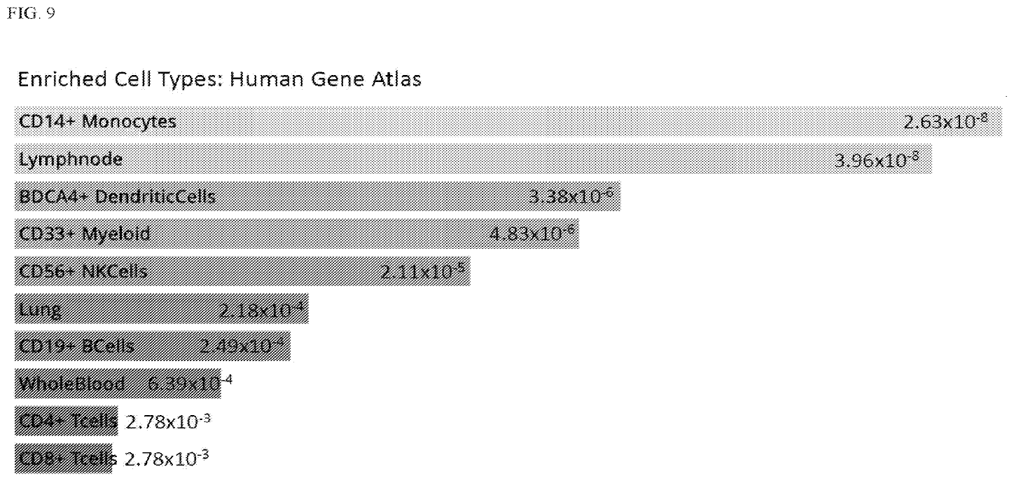

Molecular markers discriminating 24-month eGFR outcomes. A total of 595 unique genes (corresponding to 699 probe sets) were differentially expressed (FDR<0.05) in pretransplant donor organs, of which 408 were upregulated and 187 were downregulated in low function kidneys (Table 9). A volcano plot showing for all DEGs is displayed in FIG. 1A. A heatmap displaying the top shared and unique pretransplant biological pathways in low-function kidneys is depicted in FIG. 1B. These pretransplant biopsies are highly enriched in genes inducing innate (e.g., ADAM8, C1QA, CCL5, CD68, CLEC7A, HLA-F, NCKAPIL, TYROBP) and adaptive (e.g., C1QB, CD3D, CD6, CD48, CD84, GPR183, IGLL5, HLA-DQA1, HLA-DQB1, HLA-DQB2, IL71?) immune responses. Cell-type enrichment analyses identified dendritic, monocytes, myeloid, and natural killer cells as the main cell sources for the upregulated genes in pretransplant biopsies with low 24-month function (FIG. 9). In contrast, downregulated genes such as CTNND1, DLAT, ENO1, FH, GOT1, IDH2, PDS5A, RFC3 and PGK1 are involved in metabolic processes (carbon/glucose metabolism, TCA cycle), gluconeogenesis, and cell-cell adhesion, and are associated with low 24-month function.

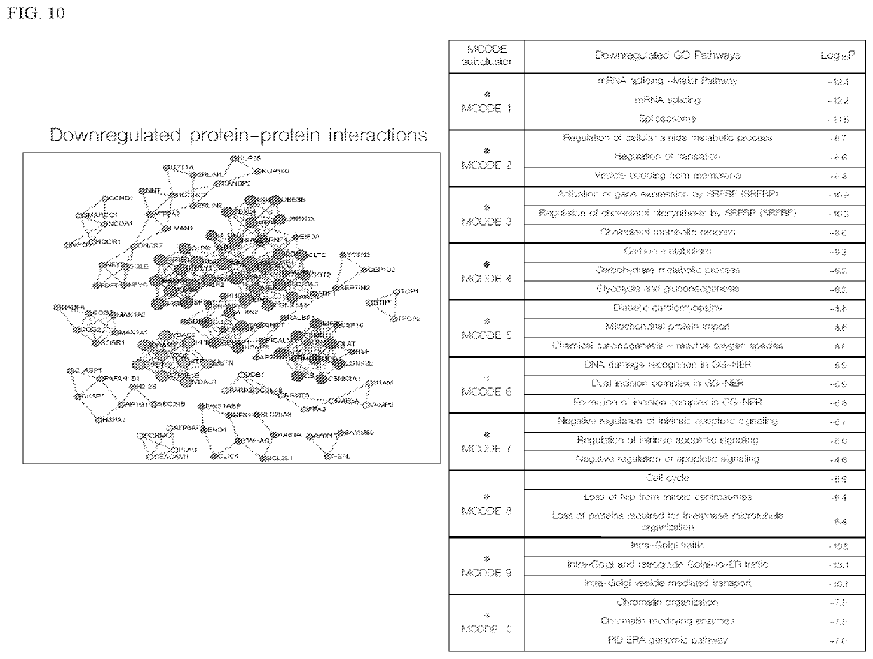

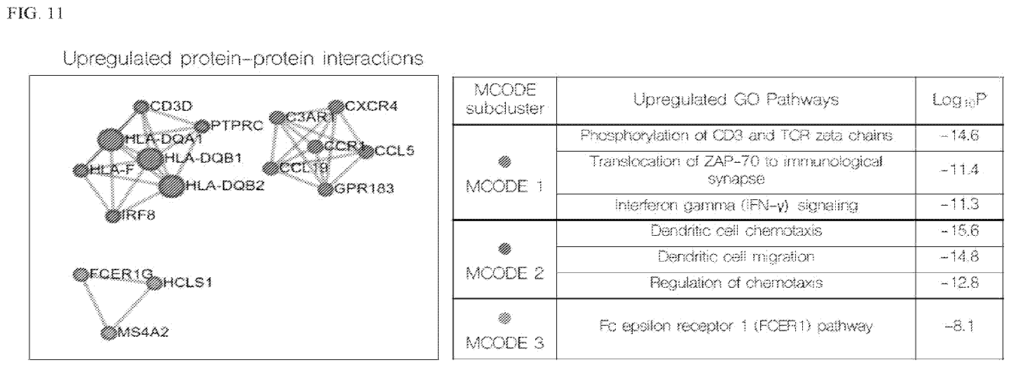

The PPIs between down- and up-regulated DEGs are displayed in FIGS. 10 and 11. Kidneys with low 24-month function exhibited many downregulated biological processes at pre-transplantation including the metabolism of cholesterol, carbon, and carbohydrates, DNA damage recognition, regulation of intrinsic apoptotic signaling, and cell cycle regulation (FIG. 10). These same kidneys showed upregulated PPI networks related to dendritic cell migration, regulation of chemotaxis, interferon gamma (IFN-γ) signaling, and the Fc epsilon receptor 1 (FCER1) pathway (FIG. 11).

### 24-Month Multivariable Models

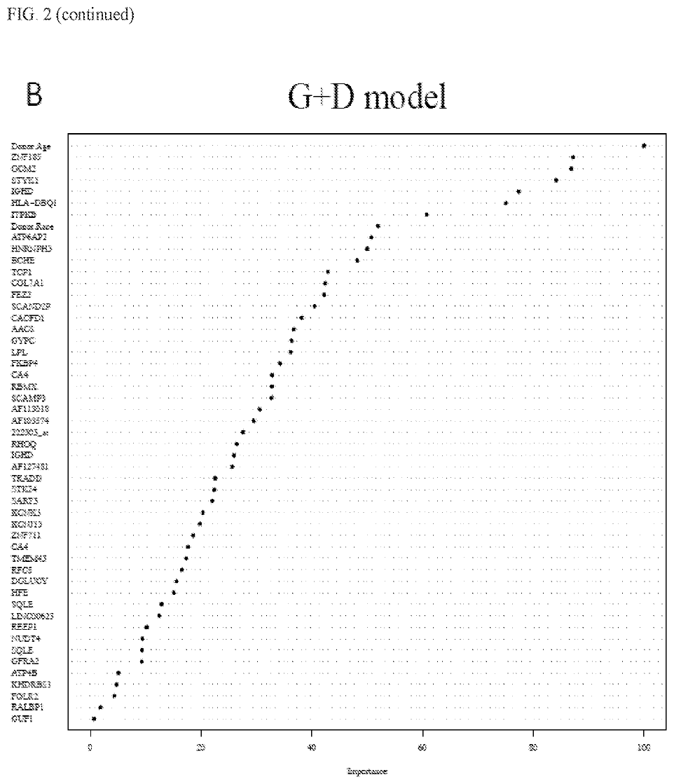

(1) Gene Expression (GE) model. When searching over the grid of parameters, the optimal value from our repeated 10-fold CV procedure was λ=0.02 and α_{LASSO}=1. When applying gene expression data (FDR≤0.05) to predict 24-month function, there were 55 significant probe sets in the penalized model (Table 2). A plot of these 55 probe sets by their variable importance is displayed in FIG. 2A. The AUROC using gene expression data (55 genes) was 0.994 (95% CI: 0.986, 1.0). When performing N-fold CV on the GE model, the AUROC was 0.767 (0.696, 0.838).

TABLE 2
GE model probe set genes.

Gene name

| | | | | |
|---|---|---|---|---|
| GCM2 | CA4 | CACFD1 | DGLUCY | KHDRBS1 |
| ZNF185 | ITPKB | ROLR2 | HFE | TMPRSS4 |
| STYK1 | LPL | IGHD | CELF2 | ZNF225 |
| FEZ2 | SCAMP3 | ITPR2 | GFRA2 | NUDT4 |
| IGHD | SYPC | TNFRSF14 | AF127481 | TCP1 |
| HLA-DQB1 | KCNK3 | RBM8A | SCAND2P | GUF1 |
| BCHE | RBMX | TRADD | ATP4B | AACS |
| FKBP4 | SART3 | MBD1 | TMEM43 | ELOA |
| HNRNPH3 | KCNJ13 | COL7A1 | RALBP1 | NEBL |
| SQLE | AF103574 | CA4 (2) | STK24 | |
| ETS2 | ATP6AP2 | REEP1 | AF113018 | |

(2) Donor Characteristics (DC) model. Donor age, race, and BMI were the only clinical characteristics significantly different when comparing the high vs. low eGFR groups (p<0.05) (Table 1). Parameter estimates, standard errors, and p-values from the DC logistic regression model are shown in Table 3. The AUROC for the training data using the three characteristics with statistical significance (donor age, race, BMI) was 0.754 (95% CI: 0.680, 0.828). The N-fold CV for the donor age, race, and BMI model is 0.727 (0.649, 0.805).

TABLE 3
Regression coefficients for the logistic regression model
that includes 3 donor characteristics (age, BMI, and race).
Lower and upper bounds of the 95% Confidence Intervals and
adjusted p-values for each regression coefficient.

| | | Lower | Upper | |
|---|---|---|---|---|
| | Coefficient | bound | bound | P-value |
| Intercept | −4.1994 | −5.8476 | −2.5512 | <0.0001 |
| Donor | 0.0463 | 0.0223 | 0.0703 | 0.0002 |
| Age | | | | |
| Donor | 0.0516 | 0.0012 | 0.1020 | 0.045 |
| Race | | | | |
| Donor | 0.7576 | 0.0171 | 1.4981 | 0.045 |
| BMI | | | | |

(3) Gene Expression+Donor Characteristics (G+D) model. When searching over the grid of parameters, the optimal values from the repeated 10-fold CV procedure were also)=0.02 and α_{LASSO}=1. When fitting the model there were 49 probe sets (Table 4) in the final model when donor age, race, and BMI were included. A plot of the 52 variables (49 probe sets and 3 donor characteristics) in order of their variable importance is displayed in FIG. 2B. The AUROC for the G+D model was 0.996 (95% CI: 0.990, 1.0). When performing the N-fold CV the AUROC was 0.809 (0.744, 0.875).

TABLE 4
G + D model probe set genes.

Gene name

| | | | | |
|---|---|---|---|---|
| ZNF185 | COL7A1 | SCAMP3 | KCNK3 | REEP1 |
| GCM2 | FEZ2 | AF113018 | KCNJ13 | NUDT4 |
| STYK1 | SCAND2P | AF103574 | ZNF711 | SQLE |
| IGHD | CACFD1 | ETS2 | CA4 (2) | GFRA2 |
| HLA-DQB1 | AACS | RHOQ | TMEM43 | ATP4B |
| ITPKB | GYPC | IGHD | RFC5 | KHDRBS1 |
| ATP6AP2 | LPL | AF127481 | DGLUCY | FOLR2 |
| HNRNPH3 | FKBP4 | TRADD | HFE | RALBP1 |
| BCHE | CA4 | STK24 | SQLE | GUF1 |
| TCP1 | RBMX | SART3 | LOC728855 | |

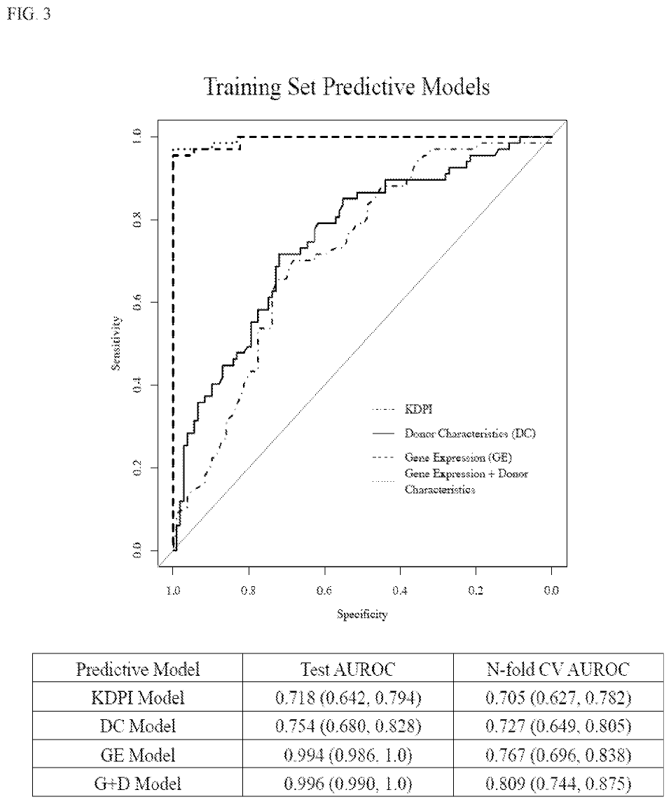

(4) KDPI model. The KDPI for each patient was calculated using 10 donor characteristics (donor age, height, weight, race, cause of death, HCV status, serum creatinine, DCD criteria, history of hypertension, and history of diabetes). Resulting numerical KDPI scores were used for the predictive model. The AUROC for the training data was 0.718 (95% CI: 0.642, 0.794). The AUROC for the N-fold CV is 0.705 (0.627, 0.782). The respective AUROC curves for the four models in the training set are shown in FIG. 3.

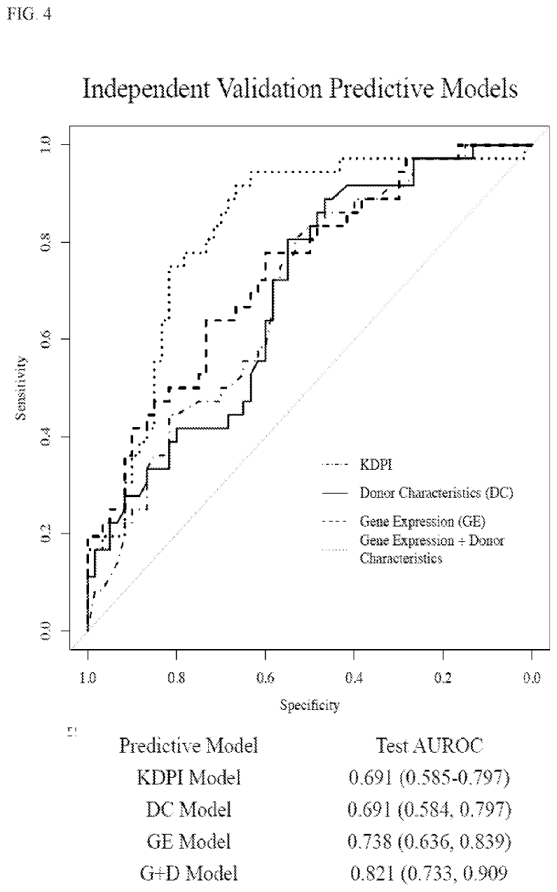

External Validation using qPCR. The validation set included 96 KT recipients, of which 36 (37.5%) had low eGFR and 60 (62.5%) had high eGFR at 24-months post-transplant (Table 5). The AUROC for the donor characteristics model (age, BMI, race) is 0.691 (95% CI: 0.584-0.797). The KDPI model calculated using 10 donor characteristics yielded the same point estimate for AUROC=0.691 (95% CI: 0.585-0.797). The 13 genes that were validated from the final models (GE and G+D) included BCHE, FKBP4, GYPC, HLA-DQB1, HNRNPH3, IGHD, NUDT4, RBM8A, RHOQ, SQLE, STK24, TRADD and ZNF185 (assay IDs provided in Table 6). The combined model (13 genes+3 donor characteristics) showed an AUROC of 0.821 (95% CI: 0.733, 0.909) for 24-month function. The respective AUROC curves for the four models after the 10-fold CV procedure are shown in FIG. 4.

TABLE 5
Characteristics of donor and recipients sub-stratified based on eGFR
at 24-month post kidney transplant in the validation set (n = 96).

| | | | | |
|---|---|---|---|---|
| | | High eGFR | Low eGFR | |
| Clinical Characteristics | Category | (n = 60) | (n = 36) | p-value |

Donor Characteristics

| Donor age (avg ± SD) | | 38.22 ± 12.65 | | 46.33 ± 13.50 | | 0.004 |
|---|---|---|---|---|---|---|
| Donor gender, | Male | 26 | (43.3) | 15 | (41.7) | 0.570 |
| n (%) | Female | 33 | (55.0) | 19 | (52.8) | |
| | Unknown | 1 | (1.7) | 2 | (5.6) | |
| Donor race, | Asian | 0 | (0.0) | 1 | (2.8) | 0.359 |
| n (%) | African American | 11 | (18.3) | 7 | (19.4) | |
| | Caucasian | 44 | (73.3) | 22 | (61.1) | |
| | Hispanic | 3 | (5.0) | 2 | (5.6) | |
| | Other | 2 | (3.3) | 4 | (11.1) | |
| DCD, n (%) | | 4 | (6.7) | 6 | (16.7) | 0.227 |
| Donor cause of death, | Anoxia | 26 | (43.3) | 12 | (33.3) | 0.241 |
| n (%) | Head trauma | 11 | (18.3) | 6 | (16.7) | |
| | Stroke | 17 | (28.3) | 17 | (47.2) | |
| | Other/Unknown | 6 | (10.0) | 1 | (2.9) | |
| Delayed graft function, | | 26 | (43.3) | 16 | (44.4) | 1.000 |
| n (%) | | | | | | |
| Donor BMI | | 29.85 | (9.06) | 36.47 | (50.16) | 0.320 |
| (avg ± SD) | | | | | | |
| CIT, hours (avg ± SD) | | 21.49 ± 10.80 | | 20.99 ± 8.02 | | 0.816 |
| WIT, min (avg ± SD) | | 35.42 | (5.23) | 33.79 | (5.83) | 0.169 |
| Pump used, n (%) | | 31 | (51.7) | 16 | (44.4) | 0.635 |
| Pump Time | | 261.25 | (356.61) | 261.83 | (383.20) | 0.994 |
| min (avg ± SD) | | | | | | |
| Last Donor Creatinine | | 1.83 | (1.71) | 1.38 | (1.18) | 0.172 |
| mg/dL (avg ± SD) | | | | | | |
| Donor HBV cAb, | Positive | 4 | (6.7) | 2 | (5.6) | 0.718 |
| n (%) | Negative | 55 | (91.7) | 34 | (94.4) | |
| | N/A | 1 | (1.7) | 0 | (0.0) | |
| Donor HCV Ab, n (%) | Positive | 19 | (31.7) | 10 | (27.8) | 0.863 |
| Donor CMV, n (%) | Positive | 29 | (48.3) | 21 | (58.3) | 0.237 |
| | Negative | 31 | (51.7) | 14 | (38.9) | |
| | N/A | 0 | (0.0) | 1 | (2.8) | |
| KDPI (avg ± SD) | | 51.68 | (23.34) | 67.36 | (20.08) | 0.001 |
| KDRI (avg ± SD) | | 1.08 | (0.33) | 1.33 | (0.46) | 0.003 |

Recipient Characteristics

| Recipient Age | | 53.33 | (12.16) | 50.64 | (11.43) | 0.290 |
|---|---|---|---|---|---|---|
| (avg ± SD) | | | | | | |
| Recipient Gender | Female | 21 | (35.0) | 9 | (25.0) | 0.571 |
| n (%) | Male | 38 | (63.3) | 26 | (72.2) | |
| | Unknown | 1 | (1.7) | 1 | (2.8) | |
| Recipient Race | African American | 42 | (70.0) | 21 | (58.3) | 0.698 |
| n (%) | Caucasian | 11 | (18.3) | 9 | (25.0) | |
| | Hispanic | 3 | (5.0) | 3 | (8.3) | |
| | Unknown | 4 | (6.7) | 3 | (8.3) | |
| Recipient BMI, | | 39.20 | (38.37) | 45.48 | (62.18) | 0.546 |
| (avg ± SD) | | | | | | |
| Recipient HCV, n (%) | Positive | 3 | (5.0) | 6 | (16.7) | 0.108 |
| | Negative | 47 | (78.3) | 27 | (75.0) | |
| | N/A | 10 | (16.7) | 3 | (8.3) | |
| CMV disease, n (%) | Positive | 6 | (10.0) | 2 | (5.6) | 0.726 |
| | Negative | 50 | (83.3) | 31 | (86.1) | |
| | N/A | 4 | (6.7) | 3 | (8.3) | |
| Recipient CMV, n (%) | Positive | 29 | (48.3) | 20 | (55.6) | 0.165 |
| | Negative | 30 | (50.0) | 13 | (36.1) | |
| | N/A | 1 | (1.7) | 3 | (8.3) | |
| Pretransplant diagnosis | DM | 11 | (18.3) | 6 | (16.7) | 0.676 |
| n (%) | DM/HTN | 10 | (16.7) | 8 | (22.2) | |
| | HTN | 14 | (23.3) | 5 | (13.9) | |
| | FSGS | 5 | (8.3) | 3 | (8.3) | |
| | Other | 18 | (30.0) | 14 | (38.9) | |
| | Unknown | 2 | (3.3) | 0 | | |
| Matched sex, n (%) | | 21 | (36.2) | 16 | (47.1) | 0.421 |
| Months on dialysis | | 45.44 ± 24.58 | | 54.22 ± 50.52 | | 0.262 |
| pretransplant | | | | | | |
| (avg ± SD) | | | | | | |

BMI: Body Mass Index; CIT: Cold Ischemia Time; CMV: Cytomegalovirus; DCD: Donation after Circulatory Death; DM: Diabetes Mellitus; FSGS: Focal Segmental Glomerulosclerosis; HBV: Hepatitis B Virus; HCV: Hepatitis C Virus; HTN: Hypertension; KDPI: Kidney Donor Profile Index; KDRI: Kidney Donor Risk Index; SCD: Standard Criteria Donor; SD Standard Deviation; WIT: Warm Ischemia Time.

TABLE 6
Gene expression assays used for qPCR validation.

| Gene name | Taqman assay ID | amplicon length |
|---|---|---|
| BCHE | Hs00163746_m1 | 64 |
| FKBP4 | Hs00427038_g1 | 105 |
| GYPC | Hs00242584_m1 | 76 |
| HLA-DQB1 | Hs03054971_m1 | 89 |
| HNRNPH3 | Hs01032113_g1 | 70 |
| IGHD | Hs00920518_g1 | 62 |
| NUDT4 | Hs01066951_g1 | 136 |
| RBM8A | Hs00254802_s1 | 69 |
| RHOQ | Hs00817629_g1 | 147 |
| SQLE | Hs01123768_m1 | 109 |
| STK24 | Hs01551911_g1 | 98 |
| TRADD | Hs00601065_g1 | 75 |
| ZNF185 | Hs00200253_m1 | 87 |

Risk Score Calculation. A 24-month graft function risk score was calculated for each patient in the independent validation cohort (n=96) based on the combined model (13 genes+3 donor characteristics). Regression coefficients, confidence intervals, and p-values are described in Table 7. Values used in the calculations are shown in Table 8. Gene expression values and donor characteristics were linearly combined into a risk score as follows, producing formula (II):

$$graft function risk score = - 4.544 + 0.29 ( Δ Ct BCHE ) + 0.023 ( Δ Ct FKBP 4 ) - 0.981 ( Δ Ct GYPC ) - 0.105 ( Δ Ct HLA - DQB 1 ) - 0.327 ( Δ Ct HNRNPH 3 ) + 0.039 ( Δ Ct IGHD ) + 0.975 ( Δ Ct NUDT 4 ) + 0.717 ( Δ Ct RBM 8 A ) - 2.182 ( Δ Ct RHOQ ) + 0.112 ( Δ Ct SQLE ) + 1.073 ( Δ Ct STK 24 ) + 0.171 ( Δ Ct TRADD ) + 0.378 ( Δ Ct ZNFI 85 ) + 0.057 ( donor age ) + 0.004 ( donor BMI ) + 0.586 ( donor race indicator variable ) \qquad ( II )$$

TABLE 7
Regression coefficients for the logistic regression model that
includes 13 genes and 3 donor characteristics (age, BMI, and
race). Lower and upper bounds of the 95% Confidence Intervals
and adjusted p-values for each regression coefficient.

| | | Lower | Upper | P- |
|---|---|---|---|---|
| | Coefficient | bound | bound | value |
| Intercept | −4.544 | −13.485 | 4.397 | 0.319 |
| Donor Age | 0.057 | 0.015 | 0.1 | 0.009 |
| Donor | 0.586 | −0.62 | 1.792 | 0.341 |
| Race | | | | |
| Donor BMI | 0.004 | −0.014 | 0.023 | 0.628 |
| BCHE | 0.29 | −0.001 | 0.581 | 0.051 |
| FKBP4 | 0.023 | −1.535 | 1.582 | 0.977 |
| GYPC | −0.981 | −1.993 | 0.032 | 0.058 |
| HLA- | −0.105 | −0.222 | 0.012 | 0.08 |
| DQB1 | | | | |
| HNRNPH3 | −0.327 | −1.982 | 1.328 | 0.698 |
| IGHD | 0.039 | −0.128 | 0.207 | 0.647 |
| NUDT4 | 0.975 | 0.131 | 1.818 | 0.024 |
| RBM8A | 0.717 | −1.522 | 2.956 | 0.53 |
| RHOQ | −2.182 | −3.885 | −0.478 | 0.012 |
| SQLE | 0.112 | −0.583 | 0.808 | 0.752 |
| STK24 | 1.073 | −0.201 | 2.346 | 0.099 |
| TRADD | 0.171 | −0.865 | 1.207 | 0.746 |
| ZNF185 | 0.378 | −0.783 | 1.539 | 0.523 |

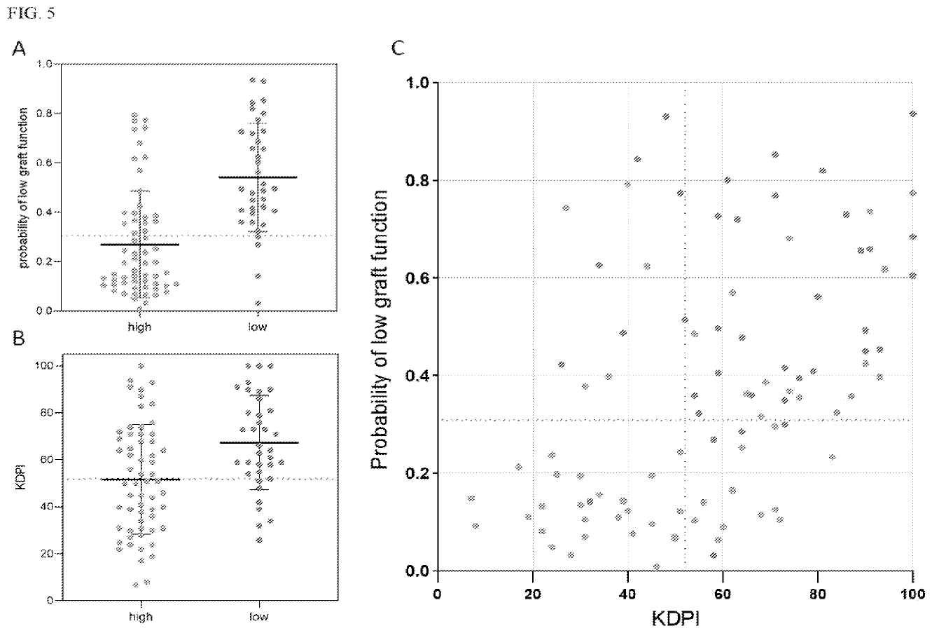

Donor race was converted to a dichotomous variable, with Caucasian=0 and all other races=1. The risk equation was then converted to a probability scale (0.0-1.0). The probability of low-graft function for each patient is plotted in FIG. 5A and the KDPI score for each patient is plotted in FIG. 5B. Youden's index was calculated for both the probability score and the KDPI, with y=0.306 and y=52 as the respective thresholds that maximize specificity and sensitivity for the validation set. When using KDPI to predict low 24-month function, the sensitivity was 80.6% and the specificity was 53.3%. When using the risk probability score, the sensitivity was 88.9% and the specificity was 66.6% (FIG. 5C).

While the invention has been described with reference to certain particular embodiments thereof, those skilled in the art will appreciate that various modifications may be made without departing from the spirit and scope of the invention. The scope of the appended claims is not to be limited to the specific embodiments described.

TABLE 8

| | | | | | | | | | | J | | | |
|---|---|---|---|---|---|---|---|---|---|---|---|---|---|
| | | | C | D | E | F | | | I | low vs. | | | |
| | A | B | donor | Donor | Donor ht | donor | G | H | 24-month | high | K | L | M |
| | Sample ID | Donor Age | race | BW (Kg) | (cm) | BMI | KDPI | KDRI | eGFR | function | NUDT | IGHD | HNRNPH3 |
| 1 | | | | | | | | | | | | | |
| 2 | 106 | 56 | 0 | 71 | 170 | 24.56747 | 73 | 1.25 | 40 | low | 2.9847612 | 16.600604 | 3.7114096 |
| 3 | 429 | 29 | 0 | 160 | 70 | 326.5306 | 61 | | 15 | low | 4.0946751 | 8.8060303 | 4.1420059 |
| 4 | 434 | 74 | 1 | 59 | 173 | 19.71332 | 90 | 1.79 | 38 | low | 2.1703272 | 8.0816641 | 3.3188181 |
| 5 | 438 | 39 | 0 | 69 | 170 | 23.87543 | 45 | 1.02 | 62 | high | 4.2520657 | 16.36055 | 3.4203243 |
| 6 | 443 | 39 | 0 | 69 | 170 | 23.87543 | 45 | | 65 | high | 3.451705 | 10.087174 | 4.0030775 |
| 7 | 444 | 38 | 0 | 74 | 155 | 30.80125 | 51 | 0.83 | 50 | high | 2.4020996 | 10.426891 | 2.996109 |
| 8 | 445 | 43 | 1 | 80 | 180 | 24.69136 | 17 | 0.71 | 65 | high | 3.9546471 | 11.371922 | 3.8861942 |
| 9 | 447 | 48 | 0 | 104 | 180 | 32.09877 | 27 | 1.2 | 56 | high | 5.0342045 | 17.406269 | 3.768589 |
| 10 | 448 | 48 | 0 | 104 | 180 | 32.09877 | 62 | 1.1 | 60 | high | 4.2872219 | 15.565096 | 4.0293217 |
| 11 | 450 | 40 | 0 | 103 | 183 | 30.75637 | 38 | 0.88 | 72 | high | 3.1051617 | 8.6642189 | 3.7784042 |
| 12 | 451 | 23 | 1 | 66 | 163 | 24.84098 | 34 | 0.91 | 54 | high | 3.3505106 | 13.82644 | 3.0685301 |
| 13 | 453 | 26 | 0 | 58 | 168 | 20.54989 | 24 | 0.78 | 62 | high | 1.7905903 | 16.968039 | 2.505537 |
| 14 | 454 | 27 | 0 | 84 | 175 | 27.42857 | 22 | 0.64 | 177 | high | 2.8171625 | 10.605376 | 2.8537893 |
| 15 | 456 | 10 | 1 | 54 | 152 | 23.37258 | 56 | 1.06 | 135 | high | 2.4126282 | 16.325363 | 2.7646008 |
| 16 | 457 | 26 | 0 | 102 | 175 | 33.30612 | 25 | 0.78 | 69 | high | 3.6084757 | 9.643117 | 3.8146563 |
| 17 | 460 | 74 | 1 | 59 | 173 | 19.71332 | 91 | 1.79 | 40 | low | 3.6190672 | 10.158198 | 3.8592844 |
| 18 | 461 | 46 | 0 | 99 | 191 | 27.13741 | 42 | | 15 | low | 3.8665152 | 16.463338 | 3.6663809 |
| 19 | 466 | 33 | 0 | 116 | 165 | 42.6079 | 50 | 0.97 | 57 | high | 2.6520271 | 15.337337 | 3.2902603 |
| 20 | 478 | 30 | 0 | 61 | 165 | 22.40588 | 22 | 0.75 | 60 | high | 4.4156771 | 11.276347 | 4.477849 |
| 21 | 479 | 32 | 1 | 107 | 173 | 35.75128 | 39 | 0.89 | 27 | low | 4.1199551 | 10.991503 | 3.8747845 |
| 22 | 480 | 65 | 0 | 80 | 183 | 23.88844 | 93 | 1.67 | 56 | high | 2.06215 | 16.958972 | 2.9586821 |
| 23 | 481 | 65 | 0 | 80 | 183 | 23.88844 | 93 | 1.67 | 38 | low | 2.1185474 | 17.179739 | 2.7983494 |
| 24 | 483 | 57 | 1 | 84 | 175 | 27.42857 | 90 | 1.47 | 37 | low | 1.2169981 | 4.5429468 | 2.1015806 |
| 25 | 485 | 27 | 1 | 89 | 173 | 29.73704 | 60 | 1.09 | 72 | high | 2.0660982 | 8.9510183 | 2.1108847 |
| 26 | 487 | 33 | 0 | 102.1 | 163 | 38.42824 | 54 | 1.03 | 83 | high | 4.3444395 | 9.500104 | 3.0953436 |
| 27 | 488 | 33 | 0 | 102.1 | 163 | 38.42824 | 54 | 1.03 | 40 | low | 3.0523949 | 15.086405 | 2.7616901 |
| 28 | 489 | 39 | 0 | 75 | 167 | 26.89232 | 59 | 1.08 | 40 | low | 3.1183786 | 8.8512869 | 2.4185228 |
| 29 | 490 | 39 | 0 | 75 | 167 | 26.89232 | 59 | 1.08 | 39 | low | 5.0701208 | 9.0529871 | 3.6517878 |
| 30 | 491 | 33 | 0 | 51 | 162 | 19.43301 | 83 | 1.47 | 50 | high | 2.8842211 | 4.3588858 | 2.8123522 |
| 31 | 493 | 55 | 0 | 103.9 | 178 | 32.79258 | 68 | 1.18 | 50 | high | 1.3907194 | 7.2602301 | 1.9226065 |
| 32 | 494 | 33 | 0 | 67.6 | 178 | 21.33569 | 64 | 1.17 | 89 | high | 4.0252581 | 9.9120016 | 3.1466074 |
| 33 | 500 | 55 | 0 | 103.9 | 178 | 32.79258 | 68 | 1.18 | 57 | high | 1.758172 | 5.9061575 | 2.4320135 |
| 34 | 501 | 46 | 1 | 95.4 | 175 | 31.15102 | 71 | 1.22 | 60 | high | 3.3992262 | 5.8387098 | 3.2104101 |
| 35 | 503 | 50 | 0 | 83.2 | 160 | 32.5 | 44 | 0.93 | 63 | high | 3.5940218 | 11.881513 | 3.3185606 |
| 36 | 509 | 33 | 0 | 95.4 | 180 | 29.44444 | 65 | 1.33 | 70 | high | 3.5910082 | 14.843657 | 2.8122644 |
| 37 | 512 | 32 | 0 | 73.9 | 178 | 23.32408 | 66 | 1.16 | 40 | low | 3.7656937 | 16.779587 | 3.7934265 |
| 38 | 513 | 29 | 1 | 104.3 | 175 | 34.05714 | 34 | 0.85 | 30 | low | 4.0419512 | 10.721375 | 2.8484545 |
| 39 | 514 | 34 | 0 | 117 | 167.6 | 41.65219 | 59 | 1.19 | 50 | high | 3.4928827 | 14.140506 | 3.3497934 |
| 40 | 515 | 39 | 0 | 117 | 152 | 50.64058 | 74 | 1.26 | 68 | high | 3.0994101 | 9.9975462 | 3.0558233 |
| 41 | 519 | 39 | 0 | 117 | 152 | 50.64058 | 52 | 1.26 | 40 | low | 3.3257847 | 6.3848267 | 2.3575764 |
| 42 | KUT 1 | 38 | 0 | 82 | 157.48 | 33.06458 | 39 | 0.89 | 50 | high | 1.1429148 | 15.465343 | 2.9228716 |
| 43 | KUT 2 | 28 | 0 | 173 | 62.6 | 441.4662 | 31 | 0.83 | 60 | high | 2.3522978 | 7.7780333 | 2.0783205 |
| 44 | KUT 4 | 28 | 0 | 62 | 173 | 20.71569 | 58 | 1.07 | 40 | low | 2.0966139 | 13.253913 | 2.7818918 |
| 45 | KUT 5 | 35 | 1 | 71 | 170 | 24.56747 | 73 | 1.25 | 38 | low | 4.2546682 | 8.8333235 | 3.3324137 |
| 46 | KUT 6 | 52 | 1 | 75 | 170 | 25.95156 | 73 | 1.25 | 39 | low | 3.7095451 | 7.3680763 | 2.9995995 |
| 47 | KUT 7 | 26 | 0 | 67 | 182 | 20.22703 | 19 | 0.73 | 60 | high | 3.3489399 | 11.29693 | 3.4193878 |
| 48 | KUT 8 | 24 | 0 | 67 | 167.6 | 23.85211 | 46 | 0.95 | 60 | high | 2.5538139 | 14.597818 | 0.8919182 |
| 49 | KUT 9 | 32 | 0 | 66 | 180 | 20.37037 | 64 | 1.14 | 38 | low | 3.3903284 | 11.841371 | 2.5802298 |
| 50 | KUT 10 | 41 | 0 | 101 | 157 | 40.97529 | 72 | 1.23 | 49 | high | 1.513422 | 3.1333847 | 2.391077 |
| 51 | KUT 12 | 42 | 1 | 144 | 183 | 42.99919 | 64 | 1.13 | 60 | high | 4.3035269 | 7.4028463 | 3.3526258 |
| 52 | KUT 13 | 32 | 1 | 100 | 180 | 30.8642 | 50 | 0.99 | 60 | high | 3.4388323 | 10.737321 | 2.6428785 |
| 53 | KUT 21 | 34 | 1 | 82 | 171 | 28.04282 | 59 | 1.09 | 35 | low | 4.0638924 | 9.4100094 | 2.8416405 |
| 54 | KUT 22 | 54 | 0 | 75 | 170.18 | 25.89669 | 55 | 1.05 | 43 | low | 3.3605442 | 11.308159 | 2.7953777 |
| 55 | KUT 24 | 28 | 1 | 107 | 193 | 28.7256 | 30 | 0.81 | 48 | high | 3.7050724 | 7.6986389 | 2.6175766 |
| 56 | KUT 27 | 28 | 0 | 78 | 185 | 22.79036 | 39 | 0.89 | 58 | high | 3.1645241 | 7.640337 | 2.3980865 |
| 57 | KUT 30 | 21 | 1 | 178 | 177 | 56.81637 | 71 | 1.22 | 59 | high | 2.4566936 | 8.5235081 | 1.509161 |
| 58 | KUT 38 | 36 | 0 | 78 | 165 | 28.65014 | 32 | 0.83 | 40 | low | 2.340662 | 11.689384 | 1.9277267 |
| 59 | KUT 48 | 42 | 0 | 63.4 | 160 | 24.76563 | 71 | 1.23 | 40 | low | 4.2249699 | 7.2232971 | 2.2242355 |
| 60 | KUT 49 | 42 | 0 | 63 | 160 | 24.60938 | 86 | 1.47 | 32 | low | 3.8613148 | 8.953764 | 2.7463322 |
| 61 | KUT 52 | 54 | 1 | 63.4 | 160 | 24.76563 | 76 | 1.3 | 60 | high | 1.028862 | 14.909374 | 2.3143806 |
| 62 | KUT 55 | 27 | 0 | 72 | 167 | 25.81663 | 31 | 0.89 | 57 | high | 1.8772526 | 9.0146599 | 1.3557339 |
| 63 | KUT 61 | 37 | 0 | 51.3 | 182.88 | 15.33857 | 28 | 0.80 | 53 | high | 3.0915718 | 9.8866634 | 2.8881645 |
| 64 | KUT 70 | 27 | 0 | 73 | 178 | 23.04002 | 62 | 1.12 | 53 | high | 4.021102 | 14.028937 | 3.3830061 |
| 65 | KUT 71 | 27 | 0 | 73 | 178 | 23.04002 | 41 | 1.12 | 54 | high | 3.3557043 | 6.6975107 | 3.3563604 |
| 66 | KUT 84 | 47 | 0 | 87.8 | 162.3 | 33.3317 | 63 | 1.13 | 39 | low | 4.4201069 | 16.331385 | 2.913929 |
| 67 | KUT 86 | 39 | 0 | 81 | 173 | 27.06405 | 76 | 1.3 | 37 | low | 4.0109825 | 8.4778843 | 3.2134647 |
| 68 | KUT 96 | 33 | 0 | 75 | 178 | 23.67125 | 71 | 1.1 | 57 | high | 2.911108 | 6.8281803 | 1.6205406 |
| 69 | KUT 102 | 33 | 0 | 102.1 | 163 | 38.42824 | 51 | 0.97 | 50 | high | 3.5895691 | 15.656218 | 4.1671753 |
| 70 | KUT 104 | 40 | 1 | 90 | 183 | 26.8745 | 54 | 1.03 | 60 | high | 1.8554115 | 13.077728 | 2.6074276 |
| 71 | KUT 110 | 52 | 1 | 75 | 170 | 25.95156 | 74 | 1.31 | 47 | high | 3.8927565 | 10.376937 | 2.9248247 |
| 72 | KUT 111 | 47 | 0 | 87 | 162 | 33.15043 | 40 | 0.9 | 60 | high | 3.2091398 | 8.9953756 | 2.7224531 |
| 73 | SEN 02 | 60 | 1 | 96.8 | 160 | 37.8125 | 100 | 2.58 | 38 | low | 4.5648708 | 15.47467 | 3.3229141 |
| 74 | SEN 03 | 58 | 1 | 95.2 | 158.2 | 38.03855 | 100 | 2.54 | 40 | low | 2.5109177 | 10.632511 | 2.6974525 |
| 75 | 10K1 | 50 | 0 | 93 | 182.88 | 27.80677 | 69 | 1.2 | 58 | high | 2.6008177 | 10.314929 | 2.6083689 |
| 76 | 21K1 | 34 | 0 | 66.68 | 165.1 | 24.46253 | 40 | 0.89 | 56 | high | 3.0144024 | 12.245018 | 2.181572 |
| 77 | 29K1 | 45 | 0 | 74 | 162.56 | 28.00299 | 36 | 0.87 | 68 | high | 5.3681183 | 12.504139 | 2.5368118 |
| 78 | 37K1 | 53 | 1 | 90.2 | 172.72 | 30.23579 | 84 | 1.45 | 94 | high | 2.4038181 | 6.0913849 | 3.546381 |
| 79 | 40K1 | 47 | 1 | 51 | 152.4 | 21.95838 | 79 | 1.34 | 38 | low | 3.688694 | 7.1889744 | 3.7682056 |
| 80 | 65K1 | 18 | 0 | 51 | 152.4 | 21.95838 | 24 | 0.77 | 77 | high | 1.9350433 | 8.6169815 | 2.5334892 |
| 81 | 68K1 | 67 | 0 | 86.5 | 165.1 | 31.73379 | 90 | 1.59 | 59 | high | 2.4254923 | 15.093215 | 2.8546247 |
| 82 | 86K1 | 41 | 0 | 75 | 172.7 | 25.14644 | 51 | 1 | 34 | low | 4.2058878 | 7.4113855 | 3.8879232 |
| 83 | 108K1 | 63 | 1 | 80 | 180 | 24.69136 | 89 | 1.55 | 35 | low | 3.1064215 | 13.822541 | 3.4051733 |
| 84 | 109K1 | 24 | 0 | 99 | 190.5 | 27.28005 | 7 | 0.63 | 71 | high | 3.3845549 | 13.973086 | 3.6638365 |
| 85 | 115K1 | 56 | 0 | 75 | 166 | 27.2173 | 87 | 1.48 | 56 | high | 3.1976442 | 6.192378 | 2.8637629 |
| 86 | 116K1 | 50 | 0 | 100 | 185 | 29.21841 | 30 | 0.81 | 57 | high | 2.5124865 | 16.857751 | 2.9671583 |
| 87 | 127K1 | 38 | 1 | 92 | 172.72 | 30.83916 | 48 | 0.97 | 21 | low | 3.7305546 | 4.9695988 | 3.2323856 |
| 88 | 134K1 | 58 | 0 | 54 | 149.86 | 24.04486 | 80 | 1.35 | 35 | low | 3.6654015 | 13.415773 | 3.3689232 |
| 89 | 166K1 | 71 | 1 | 79 | 152.4 | 34.01396 | 100 | 2.54 | 28 | low | 2.3219328 | 10.059236 | 3.7913313 |
| 90 | 172K1 | 28 | 0 | 78 | 157.48 | 31.45168 | 31 | 0.82 | 69 | high | 1.335535 | 9.0988235 | 1.9717636 |
| 91 | 173K1 | 61 | 1 | 90.7 | 188 | 25.66206 | 100 | 2.52 | 63 | high | 2.3721628 | 14.324676 | 3.2424974 |
| 92 | 177K1 | 59 | 1 | 72 | 172.72 | 24.135 | 94 | 1.74 | 67 | high | 3.150197 | 12.584586 | 2.7593451 |
| 93 | 178K1 | 29 | 0 | 66 | 175.26 | 21.48713 | 8 | 0.64 | 79 | high | 2.5163517 | 14.729034 | 2.3613415 |
| 94 | 180K1 | 61 | 1 | 81 | 152.4 | 34.87507 | 91 | 1.61 | 64 | high | 3.1220675 | 6.7587652 | 2.7979689 |
| 95 | 184K1 | 64 | 0 | 109.2 | 173 | 36.48635 | 81 | 1.38 | 39 | low | 2.4781284 | 11.53388 | 2.2424145 |
| 96 | 194K1 | 45 | 0 | 56 | 157.48 | 22.58069 | 58 | 1.07 | 17 | low | 2.8894386 | 14.095539 | 2.5252857 |
| 97 | 208K1 | 38 | 0 | 91.9 | 180.34 | 28.25735 | 26 | 0.78 | 40 | low | 1.692771 | 11.259494 | 2.4415026 |
| 98 | | | | | | | | | | | | | |

| | N | O | P | Q | R | S | T | U | V | W | X |
|---|---|---|---|---|---|---|---|---|---|---|---|
| | RHOQ | STK24 | TRADD | FKBP4 | SQLE | ZNF185 | HLA-DQB1 | GYPC | BCHE | RBM8A | NUDT*0.975 |
| 1 | | | | | | | | | | | |
| 2 | 4.8404837 | 3.8850241 | 8.6909704 | 3.6417723 | 5.7392912 | 9.2079039 | 16.600604 | 5.7837343 | 10.686087 | 5.0057154 | 2.910142207 |
| 3 | 5.1860065 | 4.266573 | 8.8607559 | 4.5417538 | 6.4815044 | 7.868763 | 4.9432449 | 5.6615658 | 12.454129 | 5.4011345 | 3.992308187 |
| 4 | 4.8519354 | 3.6285257 | 8.6702108 | 3.7400217 | 6.1907187 | 8.0468016 | 14.017325 | 4.5124388 | 7.915494 | 4.5739775 | 2.116069007 |
| 5 | 5.3112907 | 4.2799664 | 8.9572792 | 3.6789761 | 5.5549393 | 8.1740341 | 14.127296 | 7.0399513 | 10.208775 | 4.7598476 | 4.145764017 |
| 6 | 5.4957495 | 4.2998266 | 9.6198893 | 5.0739603 | 6.830699 | 8.4381456 | 11.9334 | 4.8139086 | 8.0216608 | 5.1174269 | 3.365412354 |
| 7 | 5.0047188 | 3.8812675 | 8.8350716 | 3.5612049 | 5.6700172 | 7.7428093 | 3.3305855 | 4.6835117 | 10.121126 | 4.135746 | 2.342047119 |
| 8 | 5.2478142 | 5.50844 | 8.5545616 | 3.8752098 | 6.5144424 | 8.1917648 | 13.955376 | 7.500782 | 9.0085068 | 4.800375 | 3.855780888 |
| 9 | 5.3022366 | 4.3479767 | 8.6172066 | 3.8316383 | 4.9647636 | 8.7982731 | 5.1946373 | 6.1881943 | 9.1648884 | 5.3040714 | 4.908349371 |
| 10 | 5.1027622 | 4.1948338 | 7.970808 | 3.5141964 | 5.5162601 | 8.7213612 | 5.1327667 | 6.0791264 | 8.866663 | 5.1529293 | 4.180041361 |
| 11 | 5.1666813 | 3.9872437 | 8.9676018 | 4.0998802 | 5.8054771 | 8.2909737 | 16.257412 | 5.5108337 | 11.011413 | 4.7695656 | 3.027532625 |
| 12 | 4.9539747 | 3.8507242 | 7.7283716 | 3.0845366 | 5.9819174 | 8.5879564 | 15.353044 | 5.4057341 | 10.505799 | 4.5879793 | 3.266747832 |
| 13 | 4.4213486 | 3.1509094 | 8.6169357 | 3.4209919 | 6.181242 | 7.6882496 | 16.968039 | 4.699934 | 15.443634 | 4.5391579 | 1.745825529 |
| 14 | 4.2716513 | 3.4880285 | 8.9819021 | 3.4575987 | 5.0148592 | 7.9248648 | 16.246451 | 5.4011316 | 10.361621 | 4.4549017 | 2.746733451 |
| 15 | 3.706831 | 2.6082439 | 7.4829597 | 3.5925217 | 4.7815132 | 7.2939873 | 12.996826 | 4.6713219 | 10.136199 | 4.2365246 | 2.352312469 |
| 16 | 4.3865156 | 3.6957178 | 8.5561819 | 4.007308 | 5.2468996 | 7.9138002 | 17.044728 | 5.4215803 | 10.157212 | 4.89886 | 3.518263793 |
| 17 | 5.0303392 | 4.0567274 | 8.4813223 | 3.4651766 | 5.39042 | 7.529561 | 16.279899 | 5.4607096 | 9.5519609 | 5.0174913 | 3.528590512 |
| 18 | 4.6243048 | 3.7475882 | 8.7686357 | 3.7271109 | 5.0738783 | 8.6222239 | 6.9584379 | 5.6294527 | 12.590534 | 4.9388838 | 3.769852281 |
| 19 | 5.8265324 | 5.683876 | 9.8186035 | 4.3028603 | 4.5652485 | 8.385746 | 15.337337 | 6.2381649 | 10.960086 | 4.7899132 | 2.585726452 |
| 20 | 5.0419855 | 4.1487379 | 8.8737803 | 4.4322271 | 5.5549116 | 8.3653708 | 17.014495 | 6.0548124 | 8.6574469 | 4.7480478 | 4.305285144 |
| 21 | 4.9112873 | 4.1460781 | 8.6817036 | 3.6386604 | 5.49473 | 8.4425106 | 16.591059 | 5.2404671 | 10.158973 | 5.0083752 | 4.016956186 |
| 22 | 4.3693647 | 3.2507315 | 8.1419115 | 3.1909533 | 6.3967695 | 7.1418715 | 10.610721 | 5.2451029 | 9.3193197 | 4.659503 | 2.010596251 |
| 23 | 4.2417164 | 3.1937218 | 8.1586857 | 3.1519585 | 6.3937588 | 7.3523769 | 10.20669 | 5.173708 | 8.5843248 | 4.5482826 | 2.065583754 |
| 24 | 3.5215807 | 2.4029398 | 7.6954412 | 3.0144129 | 3.1045656 | 8.2378988 | 14.081149 | 3.9358377 | 9.597311 | 3.5371237 | 1.186573148 |
| 25 | 3.6460962 | 2.1637907 | 7.8344297 | 3.1620951 | 4.4888773 | 8.3089132 | 15.271762 | 4.7522783 | 9.1968708 | 3.4692831 | 2.014445758 |
| 26 | 4.2727003 | 3.1555395 | 8.3226595 | 3.9786024 | 3.2868166 | 7.5776873 | 6.2119265 | 5.3387442 | 9.1823101 | 4.5654478 | 4.235828519 |
| 27 | 3.9522419 | 3.0456905 | 8.8221798 | 3.9690113 | 4.5278721 | 7.9539623 | 4.9264603 | 5.0584164 | 6.6768627 | 4.2584057 | 2.976084995 |
| 28 | 4.3363962 | 3.32623 | 9.1294355 | 3.8285875 | 3.3723097 | 8.5331259 | 4.8583937 | 4.2455263 | 7.3077383 | 3.9045343 | 3.040419173 |
| 29 | 4.9228106 | 3.9786654 | 9.607316 | 4.6328115 | 3.6694117 | 9.0665483 | 6.2738619 | 5.7712984 | 9.2478104 | 5.0353823 | 4.943367791 |
| 30 | 4.4529257 | 4.0970507 | 8.9781847 | 3.9118414 | 4.2897902 | 8.2442102 | 13.834771 | 5.5528269 | 12.449533 | 3.7945395 | 2.81211555 |
| 31 | 4.0697184 | 2.4462652 | 9.0916891 | 3.8756571 | 3.0013647 | 8.1431875 | 11.976756 | 3.8088198 | 11.200263 | 3.4023466 | 1.355951428 |
| 32 | 4.3534822 | 3.2426634 | 8.8553991 | 4.1444635 | 4.5872374 | 8.3092947 | 15.599578 | 5.7289019 | 9.5812082 | 4.8063231 | 3.924626613 |
| | N | O | P | Q | R | 5 | T | U | V | W | X |
| 33 | 4.4834204 | 2.9163561 | 8.6643057 | 3.50002 | 2.8890753 | 8.3969717 | 13.783389 | 4.8609114 | 9.8751297 | 4.2289324 | 1.714217734 |
| 34 | 4.4303064 | 3.8399744 | 8.324048 | 3.6461096 | 5.3559942 | 8.6545687 | 15.976911 | 4.6610289 | 10.480427 | 4.6020632 | 3.314245534 |
| 35 | 4.465539 | 3.486228 | 8.3520021 | 4.1047354 | 5.6105242 | 8.0805845 | 14.782102 | 5.2849894 | 11.971725 | 4.5331678 | 3.504171252 |
| 36 | 3.9793386 | 1.8416243 | 8.7653036 | 3.89503 | 5.010148 | 8.1372023 | 11.64096 | 4.5567999 | 10.564722 | 4.0923643 | 3.501232982 |
| 37 | 4.7446957 | 3.3986053 | 8.2956944 | 4.2885284 | 5.242321 | 7.9518204 | 5.5502567 | 5.5575409 | 10.729042 | 4.8561878 | 3.671551323 |
| 38 | 4.7660704 | 3.783534 | 8.4684076 | 3.4352312 | 4.8657484 | 8.0097647 | 6.4767065 | 5.3945475 | 11.398529 | 4.4110041 | 3.9409024 |
| 39 | 4.7992716 | 3.799098 | 8.5627623 | 4.1793966 | 5.9316511 | 8.2504187 | 13.555377 | 5.6146097 | 7.1859026 | 3.8886194 | 3.40556066 |
| 40 | 4.920146 | 4.180974 | 8.6239424 | 3.4879503 | 5.2127314 | 8.0606985 | 5.3509264 | 5.1205454 | 9.2534819 | 4.3639154 | 3.021924806 |
| 41 | 3.8817863 | 4.5719776 | 8.4519043 | 2.8560905 | 4.3382626 | 7.0102329 | 15.989407 | 5.5784264 | 9.7836065 | 3.7473488 | 3.242640066 |
| 42 | 4.2533579 | 3.0343161 | 8.6624765 | 3.8247461 | 5.4683123 | 7.6382933 | 16.733176 | 4.3144045 | 13.515029 | 3.8038664 | 1.114341903 |
| 43 | 3.7366171 | 2.4157972 | 7.2900896 | 2.9704542 | 4.2007399 | 8.1948118 | 17.322848 | 4.6915236 | 9.7517204 | 4.239831 | 2.293490338 |
| 44 | 4.8451147 | 3.0162764 | 8.1278486 | 2.8117952 | 4.7520838 | 8.1201429 | 12.345096 | 4.6367731 | 11.188543 | 3.7363653 | 2.044198537 |
| 45 | 4.8161001 | 3.5478125 | 8.0406942 | 3.7188864 | 5.1802835 | 8.4019194 | 16.039836 | 5.1529455 | 8.1391678 | 4.8543482 | 4.14830153 |
| 46 | 4.9458656 | 3.3247986 | 7.3151131 | 3.5489235 | 5.4834251 | 7.8199959 | 14.393719 | 4.5408535 | 8.3232479 | 4.4874554 | 3.616806507 |
| 47 | 5.2051163 | 3.3036461 | 8.1776924 | 4.0693684 | 4.2139721 | 8.6543941 | 5.7487354 | 5.4742985 | 11.030817 | 5.1122551 | 3.265216398 |
| 48 | 4.4436741 | 2.0473595 | 8.5670567 | 2.8500462 | 0.6542492 | 7.7659855 | 14.597818 | 5.1454887 | 9.69767 | 3.2399693 | 2.489968586 |
| 49 | 4.4739866 | 3.0033941 | 7.900569 | 3.8000956 | 5.5360861 | 8.2236338 | 4.7260389 | 5.9709253 | 13.231968 | 4.9047041 | 3.305570197 |
| 50 | 4.3960323 | 2.5004997 | 7.9486008 | 3.3166542 | 5.9414043 | 7.6849899 | 12.101631 | 3.9271736 | 10.592359 | 4.2353363 | 1.475586462 |
| 51 | 4.9116488 | 4.9469194 | 7.2832861 | 3.7329874 | 5.949357 | 7.798316 | 13.101081 | 7.0371618 | 7.3865042 | 5.1391096 | 4.195938706 |
| 52 | 4.9627752 | 2.7991457 | 7.4031115 | 3.5266218 | 5.3612375 | 7.3917627 | 10.954125 | 5.3061476 | 9.1828775 | 4.5851812 | 3.352861476 |
| 53 | 4.741333 | 2.924963 | 7.653162 | 3.946949 | 5.7420654 | 8.5416241 | 4.6637096 | 5.2371502 | 7.7768717 | 4.789093 | 3.962295055 |
| 54 | 5.1962738 | 2.9425144 | 7.8856792 | 3.5434914 | 5.1450806 | 8.592186 | 4.5907097 | 4.9642677 | 9.2739506 | 4.7488594 | 3.2765306 |
| 55 | 4.7377625 | 2.8787632 | 6.846386 | 3.1483841 | 6.2625237 | 7.935936 | 12.986069 | 4.8308907 | 8.5415993 | 5.1547623 | 3.612445593 |
| 56 | 4.4566612 | 2.8970394 | 7.3508511 | 3.6272984 | 4.2545414 | 7.7345753 | 10.458834 | 4.3317299 | 9.2226295 | 4.2018147 | 3.085410976 |
| 57 | 2.6305218 | 1.7320061 | 6.5808315 | 2.5228634 | 2.1512051 | 7.5014706 | 6.7808056 | 3.6820736 | 3.5800695 | 3.2647247 | 2.395276308 |
| 58 | 3.6735249 | 2.0646973 | 6.5472374 | 2.6702595 | 3.9314556 | 6.6829605 | 13.580631 | 3.6750507 | 9.1434631 | 3.4915733 | 2.282145452 |
| 59 | 4.560091 | 3.332716 | 7.5386238 | 3.2320518 | 4.8503742 | 7.8689346 | 1.9136562 | 4.1599426 | 10.980396 | 3.8035049 | 4.119345617 |
| 60 | 4.8703938 | 3.0405407 | 7.7050495 | 3.2949657 | 5.5698013 | 8.0570965 | 1.7703247 | 4.6668816 | 12.695679 | 4.4832172 | 3.764781904 |
| 61 | 3.8505249 | 2.2148781 | 7.6456375 | 3.2933331 | 4.5997295 | 8.0669365 | 14.909374 | 3.6970139 | 9.6209068 | 3.9547501 | 1.00314045 |
| 62 | 3.6640205 | 3.2594051 | 6.8948593 | 2.5240498 | 3.988142 | 7.6549606 | 9.9059486 | 5.5661125 | 9.7668943 | 3.6421604 | 1.830321264 |
| 63 | 5.2821274 | 3.1123657 | 7.2916279 | 3.3947411 | 6.9858875 | 7.5304813 | 8.8656483 | 5.4949074 | 10.149966 | 4.4022465 | 3.014282513 |
| 64 | 5.0837641 | 2.882226 | 7.7269926 | 4.7114115 | 5.6031084 | 8.4664412 | 0.9753866 | 5.5828848 | 8.4832029 | 5.2817163 | 3.920574403 |
| 65 | 5.4872637 | 3.1864576 | 7.471755 | 3.8006468 | 6.669919 | 8.441041 | 8.6269808 | 5.1653433 | 12.257092 | 5.0935755 | 3.2718117 |
| 66 | 4.6431999 | 3.0626259 | 7.2056065 | 3.3533478 | 6.2840958 | 8.6007824 | 16.331385 | 4.9032936 | 10.905897 | 5.0580254 | 4.309604216 |
| 67 | 4.9199333 | 2.7827435 | 7.1064243 | 3.8700542 | 5.5300751 | 7.6765804 | 3.8632336 | 4.4738369 | 10.537622 | 4.5452385 | 3.910707951 |
| 68 | 3.9636059 | 2.173872 | 8.3714104 | 3.6103954 | 3.6713486 | 7.3512306 | 15.941307 | 3.9674282 | 8.8010101 | 3.7382679 | 2.838330317 |
| 69 | 5.6814346 | 3.6957645 | 8.1726551 | 4.3545856 | 4.5506096 | 8.8946171 | 11.628824 | 5.2178421 | 11.098207 | 5.5415306 | 3.499829865 |
| 70 | 5.2240429 | 3.0860786 | 8.2483292 | 3.3388519 | 6.684267 | 7.0964966 | 13.091587 | 4.1596947 | 13.01289 | 3.8897877 | 1.809026241 |
| 71 | 4.4522562 | 2.9849024 | 7.5511122 | 3.2040834 | 5.2636938 | 7.8791018 | 11.564399 | 4.58249 | 9.0867901 | 4.2615042 | 3.795437551 |
| 72 | 4.4172373 | 2.9900503 | 8.5529356 | 3.5688772 | 4.9171419 | 8.8081961 | 4.5106649 | 4.7029104 | 11.349239 | 4.5767412 | 3.128911328 |
| 73 | 5.665781 | 5.3762474 | 9.0559845 | 4.1834087 | 4.7691326 | 6.7439976 | 9.9439468 | 6.0161572 | 15.486548 | 4.7850533 | 4.450749063 |
| 74 | 5.025444 | 3.1717796 | 9.2826805 | 3.9895172 | 4.2592316 | 8.6592159 | 10.013004 | 4.5302181 | 11.602167 | 4.4715405 | 2.448144722 |
| 75 | 4.5240583 | 2.6844778 | 7.7499533 | 3.0117826 | 3.7377176 | 8.0228796 | 12.213325 | 3.581912 | 10.494123 | 4.0907145 | 2.535797238 |
| 76 | 4.8825455 | 2.5715904 | 8.0251122 | 3.0577049 | 3.8657093 | 8.0068359 | 12.610359 | 4.2992859 | 10.865618 | 4.4899883 | 2.93904233 |
| 77 | 6.0346565 | 5.2544746 | 10.624882 | 3.0538769 | 5.7548447 | 9.1580696 | 12.504139 | 5.7377129 | 5.3379478 | 4.8705101 | 5.233915329 |
| 78 | 4.2554588 | 4.0154266 | 8.2918015 | 3.7863007 | 4.3989143 | 7.0379524 | 12.141994 | 5.2991524 | 7.8457804 | 4.4283257 | 2.343722677 |
| 79 | 4.4808102 | 4.618082 | 7.6345921 | 3.334549 | 6.8996754 | 7.9729443 | 12.449724 | 6.68083 | 6.7956181 | 5.1912289 | 3.59647665 |
| 80 | 3.5905533 | 4.2805481 | 9.1418533 | 2.7016544 | 5.2004204 | 8.1654911 | 16.323757 | 6.9940758 | 9.1762667 | 4.0863724 | 1.886667252 |
| 81 | 5.1053648 | 3.3260164 | 7.818099 | 3.3960562 | 5.9570799 | 8.2210455 | 15.093215 | 4.6656408 | 11.287598 | 4.9428778 | 2.36485498 |
| 82 | 4.2695055 | 4.1537752 | 7.4494257 | 5.283639 | 7.5772142 | 8.6587267 | 11.219998 | 3.8089056 | 1.6760826 | 5.5660295 | 4.1007406 |
| 83 | 5.1751413 | 4.0136061 | 8.4640894 | 3.4076071 | 5.6426973 | 7.9102564 | 11.418458 | 5.3755217 | 11.12338 | 4.946969 | 3.028760934 |
| 84 | 5.1224127 | 3.7330236 | 7.6938047 | 3.1801519 | 7.0318289 | 7.9006834 | 4.4125643 | 5.065011 | 9.3352203 | 4.7552786 | 3.299940991 |
| 85 | 4.8259001 | 3.2483702 | 8.2201548 | 3.2543402 | 5.5091867 | 7.8117228 | 4.7421312 | 5.0071325 | 9.4460039 | 4.0432177 | 3.117703128 |
| 86 | 3.8437815 | 2.9439936 | 7.9037714 | 3.6963415 | 3.1313524 | 7.6168127 | 16.857751 | 5.1354818 | 7.0905972 | 4.041007 | 2.449674296 |
| 87 | 4.6788807 | 4.8719082 | 6.1927242 | 2.5160122 | 6.1927242 | 6.1927242 | 1.3834972 | 2.6370678 | 5.7391224 | 4.7232189 | 3.637290716 |
| 88 | 4.6306572 | 5.0412045 | 7.9254208 | 3.4664078 | 5.9971943 | 8.0712719 | 16.6537 | 6.5381622 | 9.6589165 | 4.5920143 | 3.573766422 |
| 89 | 5.091136 | 4.0114603 | 8.7528734 | 4.0594187 | 4.841279 | 8.1309938 | 6.6686697 | 5.0175886 | 10.446953 | 4.6354799 | 2.263884473 |
| 90 | 3.6829586 | 2.133812 | 6.6971989 | 2.4376373 | 4.2509975 | 6.4840126 | 11.832287 | 3.0617752 | 8.8186607 | 3.5157871 | 1.302146673 |
| 91 | 4.2910566 | 3.169816 | 6.1635771 | 3.1947947 | 5.0116339 | 7.6724844 | 2.5648909 | 3.9064131 | 8.154808 | 4.0520849 | 2.312858748 |
| 92 | 4.3950396 | 2.9380293 | 7.220788 | 2.8971224 | 5.1786814 | 8.1741896 | 4.9788065 | 4.8974047 | 6.7537317 | 4.3945589 | 3.071442103 |
| 93 | 3.9823589 | 2.1863804 | 6.9405136 | 2.4545174 | 3.8275719 | 7.2859535 | 10.179878 | 4.8181171 | 10.503537 | 4.0852432 | 2.453442907 |
| 94 | 4.6902666 | 3.0996313 | 7.2337408 | 3.0751581 | 6.0504293 | 7.2653418 | 7.8899374 | 4.5528479 | 11.515194 | 4.3330793 | 3.044015765 |
| 95 | 4.242384 | 4.5720615 | 8.9003201 | 3.4492626 | 4.3224754 | 8.3662395 | 2.4441528 | 6.2674694 | 8.7630796 | 4.3212776 | 2.416175222 |
| 96 | 4.3316956 | 2.2713966 | 7.3341255 | 2.902998 | 3.9789848 | 7.2666492 | 8.9573669 | 4.197834 | 10.871378 | 3.8435287 | 2.817202663 |
| 97 | 4.1868353 | 3.0170584 | 8.5095701 | 3.0003977 | 4.606122 | 9.0532866 | 10.279391 | 4.2117243 | 11.482874 | 4.1540098 | 1.650451684 |
| 98 | | | | | | | | | | | |

| | Y | Z | AA | AB | AC | AD | AE | AF |
|---|---|---|---|---|---|---|---|---|
| | IGHD*0.039 | HNRNPH3*−0.327 | RHOQ*−2.182 | STK24*1.073 | TRADD*0.171 | FKBP4*0.023 | SQLE*0.112 | ZNF185*0.378 |
| 1 | | | | | | | | |
| 2 | 0.647423558 | −1.213630929 | −10.56193536 | 4.168630828 | 1.486155942 | 0.083760762 | 0.642800613 | 3.48058766 |
| 3 | 0.343435181 | −1.354435936 | −11.31586628 | 4.578032778 | 1.515189262 | 0.104460337 | 0.725928497 | 2.974392403 |
| 4 | 0.315184899 | −1.085253516 | −10.58692301 | 3.893408113 | 1.482606053 | 0.086020499 | 0.693360489 | 3.041690992 |
| 5 | 0.638061447 | −1.118446054 | −11.5892364 | 4.592403898 | 1.531694744 | 0.084616449 | 0.622153198 | 3.089784897 |
| 6 | 0.393399802 | −1.309006345 | −11.99172535 | 4.613713965 | 1.645001063 | 0.116701087 | 0.765038284 | 3.189619051 |
| 7 | 0.406648762 | −0.979727646 | −10.92029638 | 4.164600079 | 1.510797237 | 0.081907713 | 0.635041931 | 2.926781914 |
| 8 | 0.44350494 | −1.270785513 | −11.45073054 | 5.910556139 | 1.462830036 | 0.089129826 | 0.729617554 | 3.096487106 |
| 9 | 0.678844494 | −1.232328609 | −11.56948017 | 4.665378983 | 1.473542324 | 0.088127682 | 0.556053528 | 3.325747227 |
| 10 | 0.60703874 | −1.317588186 | −11.13422717 | 4.50105662 | 1.363008173 | 0.080826517 | 0.617821136 | 3.296674519 |
| 11 | 0.337904537 | −1.235538185 | −11.27369857 | 4.278312439 | 1.533459904 | 0.094297245 | 0.65021344 | 3.133988045 |
| 12 | 0.539231154 | −1.003409337 | −10.80957285 | 4.131827088 | 1.321551547 | 0.070944341 | 0.669974747 | 3.24624753 |
| 13 | 0.661753504 | −0.81931061 | −9.647382584 | 3.380925812 | 1.47349601 | 0.078682814 | 0.692299103 | 2.906158344 |
| 14 | 0.413609674 | −0.933189111 | −9.320743067 | 3.742654609 | 1.535905263 | 0.07952477 | 0.56166423 | 2.995598883 |
| 15 | 0.636689163 | −0.904024446 | −8.088305195 | 2.79864575 | 1.279586117 | 0.082627998 | 0.53552948 | 2.75712719 |
| 16 | 0.376081561 | −1.247392596 | −9.571377077 | 3.965505212 | 1.463107106 | 0.092168084 | 0.587652756 | 2.991416491 |
| 17 | 0.396169736 | −1.261985999 | −10.97620022 | 4.35286851 | 1.450306111 | 0.079699061 | 0.603727036 | 2.846174074 |
| 18 | 0.642070178 | −1.198906549 | −10.09023301 | 4.021162093 | 1.499436713 | 0.08572355 | 0.568274368 | 3.259200617 |
| 19 | 0.598156162 | −1.075915123 | −12.71349362 | 6.098798988 | 1.678981201 | 0.098965786 | 0.511307831 | 3.169811989 |
| 20 | 0.439777539 | −1.464256625 | −11.00161239 | 4.451595775 | 1.517416423 | 0.101941224 | 0.622150101 | 3.162110144 |
| 21 | 0.428668608 | −1.267054522 | −10.71642891 | 4.448741812 | 1.48457131 | 0.08368919 | 0.61540976 | 3.191269009 |
| 22 | 0.661399907 | −0.967489034 | −9.533953859 | 3.488034865 | 1.392266868 | 0.073391925 | 0.716438187 | 2.699627409 |
| 23 | 0.670009821 | −0.915060247 | −9.255425152 | 3.426863461 | 1.395135252 | 0.072495045 | 0.716100983 | 2.779198483 |
| 24 | 0.177174926 | −0.687216863 | −7.684089079 | 2.578354402 | 1.315920453 | 0.069331496 | 0.347711349 | 3.113925756 |
| 25 | 0.349089715 | −0.690259286 | −7.955781973 | 2.321747424 | 1.339687486 | 0.072728187 | 0.502754257 | 3.140769201 |
| 26 | 0.370504054 | −1.012177354 | −9.323032076 | 3.385893897 | 1.423174773 | 0.091507855 | 0.368123459 | 2.864365786 |
| 27 | 0.588369787 | −0.903072676 | −8.623791821 | 3.268025946 | 1.508592745 | 0.09128726 | 0.507121674 | 3.006597759 |
| 28 | 0.345200189 | −0.790856967 | −9.462016546 | 3.569044843 | 1.561133477 | 0.088057513 | 0.377698685 | 3.225521582 |
| 29 | 0.353066497 | −1.194134597 | −10.74157263 | 4.269107923 | 1.642851039 | 0.106554666 | 0.410974106 | 3.427155275 |
| 30 | 0.169996545 | −0.919639163 | −9.716283838 | 4.396135365 | 1.535269584 | 0.089972352 | 0.480456497 | 3.116311472 |
| 31 | 0.283148973 | −0.628692315 | −8.880125463 | 2.624842582 | 1.554678838 | 0.089140113 | 0.336152847 | 3.078124884 |
| 32 | 0.386568063 | −1.028940619 | −9.499298262 | 3.47937781 | 1.514273252 | 0.095322661 | 0.513770584 | 3.140913397 |
| 33 | 0.230340142 | −0.795268418 | −9.782823252 | 3.129250081 | 1.481596272 | 0.080500461 | 0.323576431 | 3.174055304 |
| 34 | 0.227709683 | −1.049804109 | −9.66692864 | 4.120292535 | 1.423412215 | 0.08386052 | 0.599871353 | 3.271426958 |
| 35 | 0.463378993 | −1.085169316 | −9.743806051 | 3.740722632 | 1.428192367 | 0.094408914 | 0.628378708 | 3.054460951 |
| 36 | 0.578902642 | −0.919610473 | −8.682916925 | 1.976062831 | 1.498866918 | 0.08958569 | 0.561136581 | 3.075862455 |
| 37 | 0.654403885 | −1.24045047 | −10.35292594 | 3.646703537 | 1.418563734 | 0.098636154 | 0.587139954 | 3.005788101 |
| 38 | 0.418133643 | −0.931444613 | −10.39956554 | 4.059732036 | 1.448097705 | 0.079010318 | 0.544963821 | 3.027691046 |
| 39 | 0.551479726 | −1.095382453 | −10.4720106 | 4.07643217 | 1.464232347 | 0.096126122 | 0.664344925 | 3.118658255 |
| 40 | 0.389904302 | −0.999254228 | −10.73575855 | 4.486185109 | 1.474694146 | 0.080222857 | 0.583825912 | 3.046944036 |
| 41 | 0.24900824 | −0.770927473 | −8.470057808 | 4.905731981 | 1.445275635 | 0.065690083 | 0.485885406 | 2.649868046 |
| 42 | 0.603148358 | −0.95577901 | −9.28082691 | 3.255821136 | 1.481283488 | 0.087969161 | 0.612450974 | 2.887274855 |
| 43 | 0.303343297 | −0.679610805 | −8.153298487 | 2.592150432 | 1.246605323 | 0.068320447 | 0.470482864 | 3.097638868 |
| 44 | 0.516902604 | −0.909678626 | −10.57204029 | 3.236464534 | 1.389862115 | 0.06467129 | 0.532233383 | 3.06941403 |
| 45 | 0.344499616 | −1.089699271 | −10.50873046 | 3.806802772 | 1.374958714 | 0.085534387 | 0.580191757 | 3.17592552 |
| 46 | 0.287354977 | −0.980869022 | −10.79187881 | 3.567508881 | 1.250884335 | 0.08162524 | 0.614143616 | 2.955958443 |
| 47 | 0.440580282 | −1.118139816 | −11.35756371 | 3.544812252 | 1.398385403 | 0.093595472 | 0.471964874 | 3.271360989 |
| 48 | 0.569314917 | −0.291657246 | −9.696096859 | 2.196816708 | 1.464966688 | 0.065551062 | 0.073275909 | 2.935542515 |
| 49 | 0.461813453 | −0.843735131 | −9.762238817 | 3.222641898 | 1.350997293 | 0.087402198 | 0.620041641 | 3.108533564 |
| 50 | 0.122202003 | −0.781882193 | −9.592142551 | 2.683036205 | 1.359210732 | 0.076283047 | 0.665437286 | 2.904926193 |
| 51 | 0.288711007 | −1.096308652 | −10.71721757 | 5.30804456 | 1.245441922 | 0.08585871 | 0.666327988 | 2.947763449 |
| 52 | 0.418755515 | −0.86422128 | −10.82877555 | 3.003483335 | 1.265932059 | 0.081112302 | 0.600458603 | 2.794086313 |
| 53 | 0.366990366 | −0.929216434 | −10.34558862 | 3.138485296 | 1.308690702 | 0.090779827 | 0.643111328 | 3.228733898 |
| 54 | 0.441018196 | −0.914088518 | −11.33826944 | 3.157317972 | 1.348451151 | 0.081500301 | 0.576249023 | 3.247846298 |
| 55 | 0.300246918 | −0.855947548 | −10.33779767 | 3.088912912 | 1.170731998 | 0.072412834 | 0.701402649 | 2.999783798 |
| 56 | 0.297973143 | −0.784174301 | −9.724434792 | 3.108523291 | 1.256995531 | 0.083427862 | 0.476508636 | 2.923669453 |
| 57 | 0.332416815 | −0.493495646 | −5.739798512 | 1.858442516 | 1.125322191 | 0.058025858 | 0.240934967 | 2.835555874 |
| 58 | 0.455885994 | −0.630366646 | −8.015631237 | 2.215420166 | 1.119577595 | 0.061415968 | 0.440323029 | 2.526159073 |
| 59 | 0.281708588 | −0.72732502 | −9.950118603 | 3.576004255 | 1.289104671 | 0.074337193 | 0.543241913 | 2.974457291 |
| 60 | 0.349196795 | −0.898050619 | −10.62719917 | 3.262500166 | 1.317563467 | 0.075784212 | 0.623817749 | 3.04558247 |
| 61 | 0.581465595 | −0.756802471 | −8.401845337 | 2.376564182 | 1.307404015 | 0.07574666 | 0.515169708 | 3.049301994 |
| 62 | 0.351571735 | −0.443324976 | −7.994892815 | 3.497341711 | 1.179020943 | 0.058053144 | 0.446671906 | 2.893575119 |
| 63 | 0.385579874 | −0.944429798 | −11.52560194 | 3.33956842 | 1.246868368 | 0.078079044 | 0.782419403 | 2.846521946 |
| 64 | 0.547128556 | −1.106242993 | −11.09277321 | 3.092628488 | 1.321315736 | 0.108362464 | 0.627548141 | 3.200314756 |
| 65 | 0.261202918 | −1.097529862 | −11.97320935 | 3.419069041 | 1.27767011 | 0.087414876 | 0.74703093 | 3.190713495 |
| 66 | 0.636924002 | −0.952854778 | −10.13146223 | 3.286197575 | 1.232158705 | 0.077126999 | 0.703818726 | 3.251095745 |
| 67 | 0.330637487 | −1.050802969 | −10.7352945 | 2.985883726 | 1.215198561 | 0.089011248 | 0.619368408 | 2.901747402 |
| 68 | 0.266299032 | −0.529916782 | −8.648588032 | 2.33256465 | 1.431511173 | 0.083039095 | 0.41119104 | 2.778765175 |
| 69 | 0.610592485 | −1.362666321 | −12.39689037 | 3.965555353 | 1.397524023 | 0.10015547 | 0.509668274 | 3.362165257 |
| 70 | 0.510031403 | −0.852628824 | −11.39886159 | 3.311362385 | 1.410464287 | 0.076793594 | 0.748637909 | 2.682475708 |
| 71 | 0.40470054 | −0.956417682 | −9.714823034 | 3.202800256 | 1.291240182 | 0.073693919 | 0.589533707 | 2.978300463 |
| 72 | 0.35081965 | −0.890242169 | −9.638411749 | 3.208323989 | 1.462551988 | 0.082084176 | 0.550719894 | 3.329498114 |
| 73 | 0.603512146 | −1.086592918 | −12.36273419 | 5.768713467 | 1.548573349 | 0.096218401 | 0.534142853 | 2.549231083 |
| 74 | 0.414667934 | −0.882066982 | −10.96551888 | 3.403319546 | 1.587338367 | 0.091758896 | 0.477033936 | 3.27318362 |
| 75 | 0.402282231 | −0.852936622 | −9.871495302 | 2.880444686 | 1.325242009 | 0.069271001 | 0.418624374 | 3.032648489 |
| 76 | 0.477555702 | −0.713374031 | −10.65371422 | 2.759316525 | 1.372294178 | 0.070327213 | 0.432959442 | 3.026583984 |
| 77 | 0.487661419 | −0.829537468 | −13.16762054 | 5.638051289 | 1.816854778 | 0.070239168 | 0.644542603 | 3.461750313 |
| 78 | 0.237564011 | −1.159666586 | −9.285411171 | 4.30855278 | 1.417898048 | 0.087084915 | 0.492678406 | 2.660346016 |
| 79 | 0.280370001 | −1.232203245 | −9.777127781 | 4.955202036 | 1.305515242 | 0.076694626 | 0.772763641 | 3.01377293 |
| 80 | 0.336062279 | −0.828450977 | −7.834587265 | 4.593028107 | 1.56325692 | 0.062138052 | 0.582447083 | 3.086555637 |
| 81 | 0.588635385 | −0.933462293 | −11.13990599 | 3.568815625 | 1.336894933 | 0.078109292 | 0.667192947 | 3.107555197 |
| 82 | 0.289044036 | −1.2713509 | −9.316061003 | 4.457000806 | 1.273851794 | 0.121523696 | 0.848647995 | 3.27299869 |
| 83 | 0.539079108 | −1.11349167 | −11.29215839 | 4.306599315 | 1.447359286 | 0.078374963 | 0.631982101 | 2.990076914 |
| 84 | 0.544950368 | −1.198074529 | −11.17710447 | 4.005534369 | 1.315640611 | 0.073143495 | 0.787564835 | 2.986458326 |
| 85 | 0.241502744 | −0.936450454 | −10.53011397 | 3.485501193 | 1.405646464 | 0.074849824 | 0.617028915 | 2.952831202 |
| 86 | 0.657452285 | −0.97026077 | −8.38713117 | 3.158905099 | 1.351544909 | 0.085015855 | 0.350711472 | 2.879155203 |
| 87 | 0.193814352 | −1.056990103 | −10.20931767 | 5.227557486 | 1.058955843 | 0.05786828 | 0.693585114 | 2.340849758 |
| 88 | 0.523215162 | −1.101637882 | −10.104094 | 5.409212378 | 1.35524695 | 0.079727379 | 0.67168576 | 3.050940777 |
| 89 | 0.392310187 | −1.239765332 | −11.10885871 | 4.304296906 | 1.496741355 | 0.09336663 | 0.542223251 | 3.073515673 |
| 90 | 0.354854118 | −0.644766701 | −8.036215672 | 2.289580223 | 1.145221006 | 0.056065659 | 0.476111725 | 2.450956764 |
| 91 | 0.558662347 | −1.060296664 | −9.363085573 | 3.401212586 | 1.053971681 | 0.073480277 | 0.561302994 | 2.900199102 |
| 92 | 0.49079886 | −0.902305833 | −9.589976316 | 3.152505427 | 1.234754748 | 0.066633815 | 0.580012314 | 3.089843657 |
| 93 | 0.574432343 | −0.772158663 | −8.689507191 | 2.345986155 | 1.186827827 | 0.056453899 | 0.428688049 | 2.754090431 |
| 94 | 0.263591844 | −0.914935819 | −10.23416174 | 3.325904395 | 1.236969678 | 0.070728637 | 0.677648087 | 2.746299185 |
| 95 | 0.449821329 | −0.733269533 | −9.256881794 | 4.905822031 | 1.521954729 | 0.07933304 | 0.484117249 | 3.162438549 |
| 96 | 0.549726025 | −0.825768431 | −9.451759705 | 2.437208591 | 1.254135464 | 0.066768953 | 0.445646301 | 2.746793415 |
| 97 | 0.439120259 | −0.798371341 | −9.135674601 | 3.237303634 | 1.455136491 | 0.069009147 | 0.515885666 | 3.422142317 |
| 98 | | | | | | | | |

| | AG | | | | | | | AN | AO |
|---|---|---|---|---|---|---|---|---|---|
| | HLA- | AH | Al | AJ | AK | AL | AM | coefficient = | SUM (risk |
| | DQB1*−0.105 | GYPC*−0.981 | RBM8A*0.717 | BCHE*0.29 | age*0.057 | race*0.586 | BMI*0.004 | −4.544 | score) |
| 1 | | | | | | | | | |
| 2 | −1.743063426 | −5.673843369 | 3.58909792 | 3.09896513 | 3.192 | 0 | 0.098269896 | −4.544 | −0.338638565 |
| 3 | −0.519040718 | −5.553996031 | 3.87261343 | 3.611697474 | 1.653 | 0 | 1.306122449 | −4.544 | 1.389841029 |
| 4 | −1.471819167 | −4.426702437 | 3.279541846 | 2.29549325 | 4.218 | 0.586 | 0.078853286 | −4.544 | −0.0284697 |
| 5 | −1.483366127 | −6.906192249 | 3.412810759 | 2.960544624 | 2.223 | 0 | 0.09550173 | −4.544 | −2.244905063 |
| 6 | −1.253007016 | −4.722444314 | 3.669195067 | 2.326281633 | 2.223 | 0 | 0.09550173 | −4.544 | −1.417318988 |
| 7 | −0.349711475 | −4.594525011 | 2.965329884 | 2.935126591 | 2.166 | 0 | 0.123204995 | −4.544 | −1.130774288 |
| 8 | −1.465314445 | −7.358267155 | 3.441868864 | 2.612466965 | 2.451 | 0.586 | 0.098765432 | −4.544 | −1.311089901 |
| 9 | −0.545436916 | −6.070618584 | 3.803019213 | 2.657817631 | 2.736 | 0 | 0.128395062 | −4.544 | 1.059411236 |
| 10 | 0.538940506 | −5.963622957 | 3.694650312 | 2.571332264 | 2.736 | 0 | 0.128395062 | −4.544 | 0.278465885 |
| 11 | −1.707028255 | −5.406127899 | 3.419778522 | 3.19330966 | 2.28 | 0 | 0.123025471 | −4.544 | −2.094571026 |
| 12 | −1.612069573 | −5.303025115 | 3.28958117 | 3.046681795 | 1.311 | 0.586 | 0.09936392 | −4.544 | −1.692925748 |
| 13 | −1.781644049 | −4.61063526 | 3.254576191 | 4.47865387 | 1.482 | 0 | 0.082199546 | −4.544 | −1.16640178 |
| 14 | −1.705877395 | −5.298510129 | 3.194164515 | 3.004870062 | 1.539 | 0 | 0.109714286 | −4.544 | −1.878879959 |
| 15 | −1.364666748 | −4.582566753 | 3.037588125 | 2.939497709 | 0.57 | 0.586 | 0.093490305 | −4.544 | −1.814468836 |
| 16 | −1.789696469 | −5.318570289 | 3.512482604 | 2.945591555 | 1.482 | 0 | 0.13322449 | −4.544 | −1.40354278 |
| 17 | −1.709389358 | −5.35695609 | 3.597541291 | 2.770068674 | 4.218 | 0.586 | 0.078853286 | −4.544 | 0.659466622 |
| 18 | −0.730635982 | −5.522493104 | 3.541179671 | 3.651254921 | 2.622 | 0 | 0.108549656 | −4.544 | 1.682435403 |
| 19 | −1.610420437 | −6.119639769 | 3.434367748 | 3.178424902 | 1.881 | 0 | 0.170431589 | −4.544 | −2.657496298 |
| 20 | −1.786521964 | −5.939770995 | 3.404350293 | 2.51065959 | 1.71 | 0 | 0.089623508 | −4.544 | −2.421252231 |
| 21 | −1.742061167 | −5.140898197 | 3.591004995 | 2.946102095 | 1.824 | 0.586 | 0.143005112 | −4.544 | −0.051024715 |
| 22 | −1.114125667 | −5.145445928 | 3.340863639 | 2.70260272 | 3.705 | 0 | 0.095553764 | −4.544 | −0.419238952 |
| 23 | −1.071702433 | −5.075407511 | 3.261118641 | 2.489454203 | 3.705 | 0 | 0.095553764 | −4.544 | −0.185081938 |
| 24 | −1.478520656 | −3.861056828 | 2.536117679 | 2.783220196 | 3.249 | 0.586 | 0.109714286 | −4.544 | −0.201839735 |
| 25 | −1.603534999 | −4.66198504 | 2.487475986 | 2.667092533 | 1.539 | 0.586 | 0.118948177 | −4.544 | −2.315822574 |
| 26 | −0.652252278 | −5.237308024 | 3.273426078 | 2.66286993 | 1.881 | 0 | 0.153712974 | −4.544 | −0.058362408 |
| 27 | −0.517278328 | −4.962306456 | 3.053276876 | 1.936290188 | 1.881 | 0 | 0.153712974 | −4.544 | −0.580089076 |
| 28 | −0.510131335 | −4.164861314 | 2.799551122 | 2.119244108 | 2.223 | 0 | 0.107569293 | −4.544 | −0.015426178 |
| 29 | −0.658755498 | −5.661643739 | 3.610369088 | 2.681865005 | 2.223 | 0 | 0.107569293 | −4.544 | 0.975774219 |
| 30 | −1.452650971 | −5.447323171 | 2.720684787 | 3.610364704 | 1.881 | 0 | 0.077732053 | −4.544 | −1.189858234 |
| 31 | −1.25755939 | −3.736452195 | 2.43948252 | 3.248076277 | 3.135 | 0 | 0.131170307 | −4.544 | −0.771060596 |
| 32 | −1.63795568 | −5.620052728 | 3.446133628 | 2.778550386 | 1.881 | 0 | 0.08534276 | −4.544 | −1.084368135 |
| | AG | AH | Al | AJ | AK | AL | AM | AN | AO |
| 33 | −1.447255855 | −4.768554053 | 3.032144517 | 2.863787613 | 3.135 | 0 | 0.131170307 | −4.544 | −2.042262716 |
| 34 | −1.677575612 | −4.572469314 | 3.299679299 | 3.039323769 | 2.622 | 0.586 | 0.124604082 | −4.544 | 1.201648274 |
| 35 | −1.552120671 | −5.184574559 | 3.250281341 | 3.471800108 | 2.85 | 0 | 0.13 | −4.544 | 0.506124667 |
| 36 | −1.222300773 | −4.470220691 | 2.934225211 | 3.063769398 | 1.881 | 0 | 0.117777778 | −4.544 | −0.560626375 |
| 37 | −0.582776957 | −5.451947617 | 3.481886667 | 3.111422195 | 1.824 | 0 | 0.093296301 | −4.544 | −0.578709129 |
| 38 | −0.680054183 | −5.292051061 | 3.162689916 | 3.305573425 | 1.653 | 0.586 | 0.136228571 | −4.544 | 0.514907485 |
| 39 | −1.423314586 | −5.507932134 | 2.788140126 | 2.083911753 | 1.938 | 0 | 0.166608757 | −4.544 | −2.689144927 |
| 40 | −0.561847272 | −5.023255025 | 3.128927373 | 2.683509741 | 2.223 | 0 | 0.202562327 | −4.544 | −0.542414462 |
| 41 | −1.678887691 | −5.47243626 | 2.686849079 | 2.837245893 | 2.223 | 0 | 0.202562327 | −4.544 | 0.057447524 |
| 42 | −1.756983504 | −4.232430802 | 2.727372199 | 3.919358397 | 2.166 | 0 | 0.132258329 | −4.544 | −1.782741428 |
| 43 | −1.818899074 | −4.602384604 | 3.039958806 | 2.827998924 | 1.596 | 0 | 1.765864712 | −4.544 | −0.496338957 |
| 44 | −1.296235042 | −4.54867442 | 2.678973933 | 3.244677563 | 1.596 | 0 | 0.082862775 | −4.544 | −3.414367616 |
| 45 | −1.684182773 | −5.055039554 | 3.480567647 | 2.360358658 | 1.995 | 0.586 | 0.098269896 | −4.544 | −0.845241564 |
| 46 | −1.511340466 | −4.454577284 | 3.217505499 | 2.413741894 | 2.964 | 0.586 | 0.103806228 | −4.544 | −0.62332996 |
| 47 | −0.60361722 | −5.370286806 | 3.665486904 | 3.198936939 | 1.482 | 0 | 0.080908103 | −4.544 | −2.080359931 |
| 48 | −1.532770929 | −5.047724453 | 2.323057955 | 2.812324295 | 1.368 | 0 | 0.095408434 | −4.544 | −4.718022419 |
| 49 | −0.496234088 | −5.85747775 | 3.516672835 | 3.837270699 | 1.824 | 0 | 0.081481481 | −4.544 | −0.087260528 |
| 50 | −1.270671272 | −3.852557316 | 3.03673613 | 3.071783991 | 2.337 | 0 | 0.163901172 | −4.544 | −2.14515011 |
| 51 | −1.375613494 | −6.903455752 | 3.684741591 | 2.14208621 | 2.394 | 0.586 | 0.171996775 | −4.544 | −0.919684552 |
| 52 | −1.150183167 | −5.205330771 | 3.287574946 | 2.663034487 | 1.824 | 0.586 | 0.12345679 | −4.544 | −2.591754946 |
| 53 | −0.489689512 | −5.137644339 | 3.433779694 | 2.255292788 | 1.938 | 0.586 | 0.112171266 | −4.544 | −0.381808687 |
| 54 | −0.482024517 | −4.869946644 | 3.404932194 | 2.689445667 | 3.078 | 0 | 0.103586752 | −4.544 | −0.743450964 |
| 55 | −1.363537216 | −4.739103733 | 3.695964546 | 2.477063789 | 1.596 | 0.586 | 0.114902413 | −4.544 | −1.424518714 |
| 56 | −1.098177538 | −4.249427021 | 3.012701105 | 2.674562569 | 1.596 | 0 | 0.091161432 | −4.544 | −1.793279654 |
| 57 | −0.711984587 | −3.612114195 | 2.340807632 | 1.038220167 | 1.197 | 0.586 | 0.227265473 | −4.544 | −0.866125137 |
| 58 | −1.425966282 | −3.605224771 | 2.50345808 | 2.651604309 | 2.052 | 0 | 0.114600551 | −4.544 | −1.798598719 |
| 59 | −0.200933905 | −4.080903717 | 2.727113045 | 3.184314919 | 2.394 | 0 | 0.0990625 | −4.544 | 1.759408747 |
| 60 | −0.185884094 | −4.578210812 | 3.214466761 | 3.681746826 | 2.394 | 0 | 0.0984375 | −4.544 | 0.994533155 |
| 61 | −1.565484295 | −3.626770592 | 2.835555794 | 2.790062981 | 3.078 | 0.586 | 0.0990625 | −4.544 | −0.597428816 |
| 62 | −1.040124607 | −5.46035638 | 2.611429018 | 2.832399359 | 1.539 | 0 | 0.103266521 | −4.544 | −2.140048058 |
| 63 | −0.930893068 | −5.390504139 | 3.156410723 | 2.94349021 | 2.109 | 0 | 0.061354289 | −4.544 | −3.371854159 |
| 64 | −0.102415595 | −5.476809978 | 3.786990621 | 2.460128851 | 1.539 | 0 | 0.092160081 | −4.544 | −1.626089684 |
| 65 | −0.905832982 | −5.067201762 | 3.652093617 | 3.554556818 | 1.539 | 0 | 0.092160081 | −4.544 | −2.495050369 |
| 66 | −1.714795389 | −4.810131031 | 3.626604183 | 3.162710171 | 2.679 | 0 | 0.133326804 | −4.544 | 0.945323698 |
| 67 | −0.405639524 | −4.388833998 | 3.258936001 | 3.055910511 | 2.223 | 0 | 0.108256206 | −4.544 | −0.425913492 |
| 68 | −1.673837242 | −3.892047071 | 2.680338083 | 2.552292938 | 1.881 | 0 | 0.094685015 | −4.544 | −1.93837261 |
| 69 | −1.221026545 | −5.118703102 | 3.973277447 | 3.218480167 | 1.881 | 0 | 0.153712974 | −4.544 | −1.971325019 |
| 70 | −1.374616642 | −4.080660473 | 2.788977762 | 3.77373806 | 2.28 | 0.586 | 0.107497984 | −4.544 | −2.165762197 |
| 71 | −1.21426187 | −4.495422658 | 3.055498492 | 2.635169125 | 2.964 | 0.586 | 0.103806228 | −4.544 | 0.755255217 |
| 72 | −0.473619819 | −4.613555125 | 3.281523454 | 3.291279411 | 2.679 | 0 | 0.132601738 | −4.544 | 1.337484879 |
| 73 | −1.044114418 | −5.901850164 | 3.430883183 | 4.491099043 | 3.42 | 0.586 | 0.15125 | −4.544 | 2.691080898 |
| 74 | −1.051365452 | −4.44414398 | 3.206094503 | 3.364628468 | 3.306 | 0.586 | 0.152154213 | −4.544 | 0.423228916 |
| 75 | −1.282399077 | −3.513855712 | 2.933042264 | 3.043295527 | 2.85 | 0 | 0.111227074 | −4.544 | −0.462811819 |
| 76 | −1.324087715 | −4.217599457 | 3.21932163 | 3.151029148 | 1.938 | 0 | 0.097850137 | −4.544 | −1.968495132 |
| 77 | −1.312934589 | −5.628696316 | 3.492155743 | 1.548004875 | 2.565 | 0 | 0.112011943 | −4.544 | −0.412601451 |
| 78 | −1.27490942 | −5.198468479 | 3.175109493 | 2.275276308 | 3.021 | 0.586 | 0.120943148 | −4.544 | −0.736279853 |
| 79 | −1.307221041 | −6.553894232 | 3.722111097 | 1.970729237 | 2.679 | 0.586 | 0.087833509 | −4.544 | −0.36797733 |
| 80 | −1.713994503 | −6.861188335 | 2.929928993 | 2.661117334 | 1.026 | 0 | 0.087833509 | −4.544 | −2.967185915 |
| 81 | −1.584787574 | −4.576993655 | 3.544043361 | 3.27340332 | 3.819 | 0 | 0.126935165 | −4.544 | −0.30370931 |
| 82 | −1.178099828 | −3.736536395 | 3.990843186 | 0.486063957 | 2.337 | 0 | 0.100585778 | −4.544 | 1.232252413 |
| 83 | −1.198938088 | −5.273386748 | 3.546976796 | 3.225780115 | 3.591 | 0.586 | 0.098765432 | −4.544 | 0.648780066 |
| 84 | −0.463319249 | −4.968775815 | 3.409534747 | 2.707213898 | 1.368 | 0 | 0.109120218 | −4.544 | −1.744172206 |
| 85 | −0.497923779 | −4.911997012 | 2.898987062 | 2.739341135 | 3.192 | 0 | 0.108869212 | −4.544 | −0.586224337 |
| 86 | −1.770063844 | −5.03790768 | 2.897402049 | 2.056273174 | 2.85 | 0 | 0.11687363 | −4.544 | −1.856355491 |
| 87 | −0.14526721 | −2.586963507 | 3.386547964 | 1.664345493 | 2.166 | 0.586 | 0.123356648 | −4.544 | 2.593633166 |
| 88 | −1.748638487 | −6.413937149 | 3.292474262 | 2.801085777 | 3.306 | 0 | 0.096179451 | −4.544 | 0.247226799 |
| 89 | −0.700210319 | −4.922254432 | 3.323639108 | 3.029616318 | 4.047 | 0.586 | 0.136055828 | −4.544 | 0.77356094 |
| 90 | −1.242390118 | −3.003601479 | 2.520819368 | 2.557411613 | 1.596 | 0 | 0.125806703 | −4.544 | −2.596000115 |
| 91 | −0.26931354 | −3.83219123 | 2.90534489 | 2.364894333 | 3.477 | 0.586 | 0.102648257 | −4.544 | 1.228688208 |
| 92 | −0.522774682 | −4.804353982 | 3.150898736 | 1.958582201 | 3.363 | 0.586 | 0.096539985 | −4.544 | 0.477601033 |
| 93 | −1.068887215 | −4.726572916 | 2.929119392 | 3.046025782 | 1.653 | 0 | 0.085948502 | −4.544 | −2.287110696 |
| 94 | −0.828443427 | −4.466343753 | 3.106817885 | 3.339406242 | 3.477 | 0.586 | 0.139500279 | −4.544 | 1.025997257 |
| 95 | −0.256636047 | −6.148387487 | 3.098356052 | 2.541293097 | 3.648 | 0 | 0.145945404 | −4.544 | 1.514081841 |
| 96 | −0.940523529 | −4.118075169 | 2.755810112 | 3.152699604 | 2.565 | 0 | 0.090322761 | −4.544 | −0.998812943 |
| 97 | −1.079336085 | −4.13170152 | 2.97842504 | 3.330033436 | 2.166 | 0 | 0.113029387 | −4.544 | −0.312546486 |
| 98 | | | | | | | | | |

TABLE 9

| AFFX | Symbol | FC | FDR |
|---|---|---|---|
| 221805_at | NEFL | 1.713 | 0.0498 |
| 203032_s_at | FH | 1.608 | 0.005 |
| 215236_s_at | PICALM | 1.582 | 0.0129 |
| 203548_s_at | LPL | 1.567 | 0.0491 |
| 203549_s_at | LPL | 1.52 | 0.0342 |
| 217294_s_at | ENO1 | 1.509 | 0.0105 |
| 213872_at | N/A | 1.504 | 0.0123 |
| 214279_s_at | NDRG2 | 1.496 | 0.0071 |
| 215563_s_at | MST1L | 1.482 | 0.0426 |
| 213167_s_at | SLC5A3 | 1.475 | 0.01 |
| 210165_at | DNASE1 | 1.465 | 0.0371 |
| 211538_s_at | HSPA2 | 1.46 | 0.0165 |
| 211668_s_at | PLAU | 1.449 | 0.0166 |
| 211150_s_at | DLAT | 1.419 | 1.00E−04 |
| 201337_s_at | VAMP3 | 1.417 | 0.017 |
| 210735_s_at | CA12 | 1.415 | 0.0032 |
| 212281_s_at | TMEM97 | 1.406 | 0.0176 |
| 202784_s_at | NNT | 1.385 | 0.005 |
| 201835_s_at | PRKAB1 | 1.378 | 0.0146 |
| 220324_at | LINC00472 | 1.361 | 0.0362 |
| 214359_s_at | HSP90AB1 | 1.358 | 0.0088 |
| 203962_s_at | NEBL | 1.355 | 7.00E−04 |
| 201490_s_at | PPIF | 1.353 | 0.0042 |
| 203641_s_at | COBLL1 | 1.345 | 0.0165 |
| 212183_at | NUDT4 | 1.34 | 0.0105 |
| 203293_s_at | LMAN1 | 1.338 | 0.0146 |
| 219929_s_at | ZFYVE21 | 1.337 | 0.027 |
| 209218_at | SQLE | 1.335 | 0.0156 |
| 214691_x_at | MINDY2 | 1.333 | 0.0088 |
| 208750_s_at | ARF1 | 1.33 | 0.0335 |
| 201790_s_at | DHCR7 | 1.329 | 0.0031 |
| 207549_x_at | CD46 | 1.328 | 0.0444 |
| 210403_s_at | KCNJ1 | 1.322 | 0.0341 |
| 212282_at | TMEM97 | 1.322 | 0.0226 |
| 212568_s_at | DLAT | 1.322 | 1.00E−04 |
| 212595_s_at | DAZAP2 | 1.322 | 0.0064 |
| 212787_at | YLPM1 | 1.318 | 0.0032 |
| 210935_s_at | WDR1 | 1.317 | 0.0346 |
| 214581_x_at | TNFRSF21 | 1.317 | 0.0225 |
| 200866_s_at | PSAP | 1.316 | 0.0107 |
| 218434_s_at | AACS | 1.314 | 0.0042 |
| 208712_at | CCND1 | 1.31 | 0.0186 |
| 201559_s_at | CLIC4 | 1.309 | 0.0494 |
| 210649_s_at | ARID1A | 1.305 | 0.0186 |
| 211450_s_at | MSH6 | 1.303 | 0.022 |
| 220999_s_at | CYFIP2 | 1.302 | 0.0494 |
| 217744_s_at | PERP | 1.301 | 0.0252 |
| 208116_s_at | MAN1A1 | 1.3 | 0.0286 |
| 211574_s_at | CD46 | 1.297 | 0.0382 |
| 213562_s_at | SQLE | 1.297 | 0.0086 |
| 214959_s_at | API5 | 1.295 | 0.0149 |
| 212009_s_at | STIP1 | 1.293 | 0.029 |
| 204149_s_at | GSTM4 | 1.288 | 0.0346 |
| 209627_s_at | OSBPL3 | 1.285 | 0.0185 |
| 213110_s_at | COL4A5 | 1.285 | 0.0225 |
| 206893_at | SALL1 | 1.283 | 0.0157 |
| 209772_s_at | CD24 | 1.283 | 0.0376 |
| 212507_at | TMEM131 | 1.282 | 0.0047 |
| 203961_at | NEBL | 1.28 | 0.007 |
| 220477_s_at | TMEM230 | 1.28 | 0.0225 |
| 207627_s_at | TFCP2 | 1.279 | 0.0031 |
| 219675_s_at | UXS1 | 1.277 | 0.0165 |
| 206302_s_at | NUDT4 | 1.276 | 0.0426 |
| 216899_s_at | SKAP2 | 1.275 | 0.0195 |
| 219204_s_at | SRR | 1.275 | 0.0042 |
| 204367_at | SP2 | 1.273 | 0.0094 |
| 215714_s_at | SMARCA4 | 1.271 | 0.0086 |
| 211681_s_at | PDLIM5 | 1.27 | 0.0419 |
| 211689_s_at | TMPRSS2 | 1.27 | 0.005 |
| 212266_s_at | SRSF5 | 1.27 | 0.0175 |
| 210832_x_at | PTGER3 | 1.269 | 0.0282 |
| 202593_s_at | GDE1 | 1.265 | 0.0107 |
| 213658_at | N/A | 1.265 | 0.0318 |
| 212305_s_at | MIA3 | 1.264 | 0.0024 |
| 202539_s_at | HMGCR | 1.262 | 0.0177 |
| 203634_s_at | CPT1A | 1.262 | 0.0417 |
| 213122_at | TSPYL5 | 1.262 | 0.0185 |
| 200722_s_at | CAPRIN1 | 1.26 | 0.0151 |
| 221761_at | ADSS2 | 1.26 | 0.0346 |
| 206245_s_at | IVNS1ABP | 1.258 | 0.0331 |
| 208675_s_at | DDOST | 1.258 | 0.0246 |
| 221871_s_at | TFG | 1.258 | 0.0082 |
| 210154_at | ME2 | 1.256 | 0.0208 |
| 212325_at | LIMCH1 | 1.256 | 0.0047 |
| 210655_s_at | FOXO3 | 1.255 | 0.0416 |
| 204032_at | BCAR3 | 1.253 | 0.0089 |
| 202783_at | NNT | 1.252 | 0.0042 |
| 206113_s_at | RAB5A | 1.251 | 0.0494 |
| 202444_s_at | ERLIN1 | 1.249 | 0.0367 |
| 214720_x_at | SEPTIN10 | 1.248 | 0.0212 |
| 217188_s_at | ERG28 | 1.248 | 0.0185 |
| 204156_at | SIK3 | 1.246 | 0.0185 |
| 209925_at | OCLN | 1.246 | 0.044 |
| 212599_at | AUTS2 | 1.244 | 0.0138 |
| 212388_at | USP24 | 1.243 | 0.0028 |
| 34764_at | LARS2 | 1.243 | 0.0108 |
| 207622_s_at | ABCF2 | 1.242 | 0.0054 |
| 212093_s_at | MTUS1 | 1.242 | 0.0495 |
| 217356_s_at | PGK 1.00 | 1.242 | 0.0063 |
| 202242_at | TSPAN7 | 1.241 | 0.0229 |
| 204361_s_at | SKAP2 | 1.241 | 0.0186 |
| 213461_at | NUDT21 | 1.241 | 0.0168 |
| 214889_at | FAM149A | 1.239 | 0.0485 |
| 211852_s_at | ATRN | 1.238 | 0.0426 |
| 212335_at | GNS | 1.238 | 0.005 |
| 221669_s_at | ACAD8 | 1.238 | 0.0304 |
| 201634_s_at | CYB5B | 1.237 | 0.0072 |
| 211749_s_at | VAMP3 | 1.237 | 0.029 |
| 203723_at | ITPKB | 1.236 | 0.0042 |
| 203116_s_at | FECH | 1.235 | 0.0201 |
| 220773_s_at | GPHN | 1.235 | 0.0346 |
| 200894_s_at | FKBP4 | 1.234 | 0.0188 |
| 201259_s_at | SYPL1 | 1.234 | 0.0107 |
| 200790_at | ODC1 | 1.233 | 0.005 |
| 200918_s_at | SRPRA | 1.232 | 0.0084 |
| 212392_s_at | PDE4DIP | 1.232 | 0.0473 |
| 201075_s_at | SMARCC1 | 1.231 | 0.0278 |
| 221487_s_at | ENSA | 1.231 | 0.0129 |
| 205273_s_at | PITRM1 | 1.23 | 0.005 |
| 208453_s_at | XPNPEP1 | 1.23 | 0.0185 |
| 214101_s_at | NPEPPS | 1.229 | 0.0381 |
| 200778_s_at | SEPTIN2 | 1.227 | 0.0322 |
| 209186_at | ATP2A2 | 1.226 | 0.029 |
| 200923_at | LGALS3BP | 1.225 | 0.0346 |
| 208694_at | PRKDC | 1.225 | 0.0061 |
| 211797_s_at | NFYC | 1.225 | 0.0242 |
| 217805_at | ILF3 | 1.225 | 0.0146 |
| 201131_s_at | CDH1 | 1.224 | 0.0252 |
| 221550_at | COX15 | 1.224 | 0.0042 |
| 202601_s_at | HTATSF1 | 1.223 | 0.0426 |
| 210793_s_at | NUP98 | 1.223 | 0.0089 |
| 215030_at | GRSF1 | 1.22 | 0.0063 |
| 205761_s_at | DUS4L | 1.219 | 0.0261 |
| 215471_s_at | MAP7 | 1.219 | 0.0185 |
| 202101_s_at | RALB | 1.218 | 0.0097 |
| 205822_s_at | HMGCS1 | 1.217 | 0.0387 |
| 216511_s_at | TCF7L2 | 1.217 | 0.0375 |
| 212193_s_at | LARP1 | 1.216 | 0.0494 |
| 200883_at | UQCRC2 | 1.215 | 0.0042 |
| 209624_s_at | MCCC2 | 1.215 | 0.0097 |
| 222258_s_at | SH3BP4 | 1.215 | 0.0156 |
| 212154_at | SDC2 | 1.214 | 0.0218 |
| 204312_x_at | CREB1 | 1.213 | 0.0313 |
| 210658_s_at | GGA2 | 1.213 | 0.0064 |
| 212381_at | USP24 | 1.213 | 0.0082 |
| 203209_at | RFC5 | 1.212 | 0.0105 |
| 204040_at | RNF144A | 1.212 | 0.0492 |
| 210046_s_at | IDH2 | 1.211 | 0.0187 |
| 205895_s_at | NOLC1 | 1.21 | 0.0031 |
| 219121_s_at | ESRP1 | 1.21 | 0.0147 |
| 201929_s_at | PKP4 | 1.209 | 0.0393 |
| 211812_s_at | B3GALNT1 | 1.209 | 0.0387 |
| 218129_s_at | NFYB | 1.209 | 0.0444 |
| 202662_s_at | ITPR2 | 1.208 | 0.0107 |
| 202966_at | CAPN6 | 1.207 | 0.0261 |
| 208953_at | LARP4B | 1.207 | 0.0386 |
| 209036_s_at | MDH2 | 1.206 | 0.0024 |
| 201120_s_at | PGRMC1 | 1.205 | 0.0494 |
| 202226_s_at | CRK | 1.205 | 0.0146 |
| 203544_s_at | STAM | 1.205 | 0.0185 |
| 208854_s_at | STK24 | 1.205 | 0.0188 |
| 213302_at | PFAS | 1.205 | 0.0061 |
| 210949_s_at | EIF3CL | 1.204 | 0.0146 |
| 210976_s_at | PFKM | 1.204 | 0.0042 |
| 201370_s_at | CUL3 | 1.203 | 0.0137 |
| 217860_at | NDUFA10 | 1.203 | 0.0095 |
| 218331_s_at | TASOR2 | 1.202 | 0.0434 |
| 200606_at | DSP | 1.201 | 0.0043 |
| 201378_s_at | UBAP2L | 1.2 | 0.007 |
| 212164_at | TMEM183A | 1.2 | 0.0275 |
| 213149_at | DLAT | 1.2 | 0.0131 |
| 218595_s_at | HEATR1 | 1.2 | 0.0259 |
| 200927_s_at | RAB14 | 1.199 | 0.0231 |
| 204468_s_at | TIE1 | 1.199 | 0.0466 |
| 205732_s_at | NCOA2 | 1.199 | 0.0376 |
| 215832_x_at | PICALM | 1.199 | 0.0223 |
| 211337_s_at | TUBGCP4 | 1.198 | 0.0227 |
| 216202_s_at | SPTLC2 | 1.198 | 0.0446 |
| 203210_s_at | RFC5 | 1.197 | 0.007 |
| 207791_s_at | RAB1A | 1.197 | 0.0151 |
| 208407_s_at | CTNND1 | 1.197 | 0.0042 |
| 208459_s_at | XPO7 | 1.197 | 0.0421 |
| 218716_x_at | MTO1 | 1.197 | 0.0183 |
| 202845_s_at | RALBP1 | 1.196 | 0.0024 |
| 209105_at | NCOA1 | 1.196 | 0.0042 |
| 201383_s_at | NBR1 | 1.195 | 0.0385 |
| 211323_s_at | ITPR1 | 1.195 | 0.0416 |
| 212377_s_at | NOTCH2 | 1.194 | 0.0146 |
| 218342_s_at | ERMP1 | 1.194 | 0.0361 |
| 221542_s_at | ERLIN2 | 1.194 | 0.0275 |
| 201052_s_at | PSMF1 | 1.193 | 0.0154 |
| 202179_at | BLMH | 1.191 | 0.0486 |
| 204832_s_at | BMPR1A | 1.191 | 0.0146 |
| 207781_s_at | ZNF711 | 1.191 | 0.0346 |
| 208308_s_at | GPI | 1.191 | 0.0108 |
| 208990_s_at | HNRNPH3 | 1.191 | 0.0054 |
| 212310_at | MIA3 | 1.191 | 0.0476 |
| 218827_s_at | CEP192 | 1.191 | 0.0183 |
| 201169_s_at | BHLHE40 | 1.19 | 0.0399 |
| 201444_s_at | ATP6AP2 | 1.189 | 0.0322 |
| 204796_at | EML1 | 1.189 | 0.0466 |
| 215549_x_at | CTAGE9 | 1.189 | 0.0291 |
| 204128_s_at | RFC3 | 1.188 | 0.0138 |
| 217725_x_at | SERBP1 | 1.188 | 0.0084 |
| 220238_s_at | KLHL7 | 1.188 | 0.0175 |
| 201662_s_at | ACSL3 | 1.187 | 0.0072 |
| 210480_s_at | MYO6 | 1.186 | 0.0494 |
| 212709_at | NUP160 | 1.186 | 0.0434 |
| 213260_at | FOXC1 | 1.186 | 0.0346 |
| 218172_s_at | DERL1 | 1.186 | 0.0386 |
| 207275_s_at | ACSL1 | 1.185 | 0.0232 |
| 218917_s_at | ARID1A | 1.185 | 0.0491 |
| 201048_x_at | RAB6A | 1.184 | 0.0314 |
| 201686_x_at | API5 | 1.184 | 0.0311 |
| 210543_s_at | PRKDC | 1.184 | 0.0338 |
| 218156_s_at | TSR1 | 1.184 | 0.0105 |
| 200723_s_at | CAPRIN1 | 1.183 | 0.0082 |
| 208351_s_at | MAPK1 | 1.183 | 0.0437 |
| 212482_at | RMND5A | 1.183 | 0.0187 |
| 218659_at | ASXL2 | 1.182 | 0.0293 |
| 36830_at | MIPEP | 1.182 | 0.0446 |
| 203428_s_at | ASF1A | 1.181 | 0.0498 |
| 209943_at | FBXL4 | 1.181 | 0.0303 |
| 212415_at | SEPTIN6 | 1.181 | 0.0372 |
| 200687_s_at | SF3B3 | 1.18 | 0.005 |
| 205094_at | PEX12 | 1.18 | 0.0363 |
| 209236_at | SLC23A2 | 1.18 | 0.0261 |
| 210191_s_at | PHTF1 | 1.18 | 0.0232 |
| 210257_x_at | CUL4B | 1.18 | 0.0485 |
| 216457_s_at | SF3A1 | 1.18 | 0.0363 |
| 207809_s_at | ATP6AP1 | 1.179 | 0.0294 |
| 200793_s_at | ACO2 | 1.178 | 0.0346 |
| 203194_s_at | NUP98 | 1.178 | 0.0186 |
| 212506_at | PICALM | 1.178 | 0.0293 |
| 214934_at | ATP9B | 1.178 | 0.0376 |
| 219217_at | NARS2 | 1.178 | 0.0356 |
| 219433_at | BCOR | 1.178 | 0.0382 |
| 200613_at | AP2M1 | 1.177 | 0.0434 |
| 201799_s_at | OSBP | 1.177 | 0.0054 |
| 208773_s_at | ANKHD1 | 1.176 | 0.0057 |
| 208310_s_at | CCZ1B | 1.175 | 0.0417 |
| 208666_s_at | ST13 | 1.175 | 0.0257 |
| 208813_at | GOT1 | 1.175 | 0.0107 |
| 215424_s_at | SNW1 | 1.175 | 0.0042 |
| 222036_s_at | MCM4 | 1.175 | 0.0365 |
| 214113_s_at | RBM8A | 1.174 | 0.0056 |
| 218884_s_at | GUF1 | 1.174 | 0.0346 |
| 202889_x_at | MAP7 | 1.173 | 0.0324 |
| 212038_s_at | VDAC1 | 1.173 | 0.0186 |
| 213280_at | RAP1GAP2 | 1.173 | 0.041 |
| 214136_at | NUDT13 | 1.173 | 0.0417 |
| 218594_at | HEATR1 | 1.173 | 0.024 |
| 212752_at | CLASP1 | 1.172 | 0.0437 |
| 214462_at | SOCS6 | 1.172 | 0.0126 |
| 200647_x_at | EIF3CL | 1.171 | 0.0231 |
| 216944_s_at | ITPR1 | 1.171 | 0.0331 |
| 207776_s_at | CACNB2 | 1.17 | 0.0082 |
| 202717_s_at | CDC16 | 1.169 | 0.0157 |
| 203963_at | CA12 | 1.169 | 0.0363 |
| 208683_at | CAPN2 | 1.169 | 0.0042 |
| 209137_s_at | USP10 | 1.169 | 0.0115 |
| 218174_s_at | TMEM254 | 1.169 | 0.0363 |
| 207821_s_at | PTK2 | 1.168 | 0.0396 |
| 211941_s_at | PEBP1 | 1.168 | 0.0426 |
| 214908_s_at | TRRAP | 1.168 | 0.0238 |
| 217300_at | U80771 | 1.168 | 0.0494 |
| 217786_at | PRMT5 | 1.168 | 0.0072 |
| 200615_s_at | AP2B1 | 1.167 | 0.0457 |
| 200662_s_at | TOMM20 | 1.167 | 0.024 |
| 201529_s_at | RPA1 | 1.167 | 0.0492 |
| 218170_at | ISOC1 | 1.167 | 0.0327 |
| 219081_at | ANKHD1 | 1.167 | 0.0129 |
| 219581_at | TSEN2 | 1.167 | 0.0183 |
| 202529_at | PRPSAP1 | 1.165 | 0.0082 |
| 207079_s_at | MED6 | 1.165 | 0.0494 |
| 209535_s_at | AF127481 | 1.164 | 0.0282 |
| 210950_s_at | FDFT1 | 1.164 | 0.0378 |
| 216035_x_at | TCF7L2 | 1.164 | 0.0497 |
| 200764_s_at | CTNNA1 | 1.163 | 0.0089 |
| 203801_at | MRPS14 | 1.163 | 0.0371 |
| 208598_s_at | HUWE1 | 1.163 | 0.0084 |
| 219374_s_at | ALG9 | 1.163 | 0.0311 |
| 204593_s_at | MIEF1 | 1.162 | 0.0061 |
| 211997_x_at | H3-3B | 1.162 | 0.0352 |
| 220735_s_at | SENP7 | 1.162 | 0.0468 |
| 202085_at | TJP2 | 1.16 | 0.0397 |
| 203688_at | PKD2 | 1.16 | 0.027 |
| 200753_x_at | SRSF2 | 1.159 | 0.0366 |
| 201576_s_at | GLB1 | 1.159 | 0.0165 |
| 203350_at | AP1G1 | 1.159 | 0.0252 |
| 212260_at | GIGYF2 | 1.159 | 0.0186 |
| 212407_at | EEF1AKNMT | 1.159 | 0.0084 |
| 217980_s_at | MRPL16 | 1.158 | 0.024 |
| 215230_x_at | EIF3C | 1.157 | 0.0363 |
| 201118_at | PGD | 1.156 | 0.0286 |
| 212428_at | ECPAS | 1.156 | 0.0037 |
| 212186_at | ACACA | 1.155 | 0.0387 |
| 214086_s_at | PARP2 | 1.155 | 0.025 |
| 201054_at | HNRNPA0 | 1.154 | 0.0201 |
| 207058_s_at | PRKN | 1.154 | 0.0409 |
| 208883_at | UBR5 | 1.154 | 0.0417 |
| 211139_s_at | NAB1 | 1.154 | 0.0442 |
| 212272_at | LPIN1 | 1.154 | 0.0271 |
| 212711_at | CAMSAP1 | 1.154 | 0.0301 |
| 213021_at | GOSR1 | 1.154 | 0.0129 |
| 217842_at | LUC7L2 | 1.154 | 0.042 |
| 203972_s_at | PEX3 | 1.153 | 0.048 |
| 206200_s_at | ANXA11 | 1.153 | 0.0412 |
| 212025_s_at | FLII | 1.153 | 0.034 |
| 218298_s_at | DGLUCY | 1.153 | 0.0265 |
| 218689_at | FANCF | 1.153 | 0.0129 |
| 221958_s_at | WLS | 1.153 | 0.0408 |
| 201685_s_at | TOX4 | 1.152 | 0.0063 |
| 208631_s_at | HADHA | 1.151 | 0.0208 |
| 200684_s_at | UBE2L3 | 1.15 | 0.048 |
| 212121_at | TCTN3 | 1.15 | 0.0105 |
| 201903_at | UQCRC1 | 1.149 | 0.0376 |
| 208643_s_at | XRCC5 | 1.149 | 0.0175 |
| 211404_s_at | APLP2 | 1.149 | 0.0181 |
| 212403_at | UBE3B | 1.149 | 0.0231 |
| 216338_s_at | YIPF3 | 1.148 | 0.0379 |
| 200828_s_at | ZNF207 | 1.147 | 0.0303 |
| 201928_at | PKP4 | 1.147 | 0.0363 |
| 202798_at | SEC24B | 1.147 | 0.0268 |
| 214248_s_at | TRIM2 | 1.147 | 0.0264 |
| 218289_s_at | UBA5 | 1.147 | 0.0188 |
| 205300_s_at | SNRNP35 | 1.146 | 0.0296 |
| 218177_at | CHMP1B | 1.146 | 0.0428 |
| 200040_at | KHDRBS1 | 1.145 | 0.0104 |
| 200657_at | SLC25A5 | 1.145 | 0.0187 |
| 202097_at | NUP153 | 1.145 | 0.0446 |
| 206015_s_at | FOXJ3 | 1.145 | 0.0063 |
| 207546_at | ATP4B | 1.145 | 0.0185 |
| 212644_s_at | MAPK1IP1L | 1.145 | 0.0286 |
| 217759_at | TRIM44 | 1.145 | 0.0188 |
| 200597_at | EIF3A | 1.144 | 0.0497 |
| 212133_at | NIPA2 | 1.143 | 0.0156 |
| 220367_s_at | SAP130 | 1.143 | 0.0443 |
| 201112_s_at | CSE1L | 1.142 | 0.0321 |
| 205417_s_at | DAG1 | 1.142 | 0.0443 |
| 201528_at | RPA1 | 1.141 | 0.0479 |
| 202374_s_at | RAB3GAP2 | 1.141 | 0.0249 |
| 212141_at | MCM4 | 1.141 | 0.0479 |
| 201570_at | SAMM50 | 1.14 | 0.0168 |
| 208649_s_at | VCP | 1.14 | 0.0378 |
| 220486_x_at | TMEM164 | 1.14 | 0.0491 |
| 201093_x_at | SDHA | 1.139 | 0.0147 |
| 208021_s_at | RFC1 | 1.139 | 0.0165 |
| 214988_s_at | SON | 1.139 | 0.0216 |
| 217828_at | SLTM | 1.139 | 0.0434 |
| 208777_s_at | PSMD11 | 1.138 | 0.0381 |
| 203978_at | NUBP1 | 1.137 | 0.0225 |
| 208855_s_at | STK24 | 1.137 | 0.0129 |
| 211255_x_at | DEDD | 1.137 | 0.0453 |
| 212729_at | DLG3 | 1.137 | 0.0149 |
| 203073_at | COG2 | 1.136 | 0.024 |
| 208674_x_at | DDOST | 1.136 | 0.0296 |
| 200609_s_at | WDR1 | 1.135 | 0.0084 |
| 217857_s_at | RBM8A | 1.135 | 0.0107 |
| 201086_x_at | SON | 1.134 | 0.0138 |
| 202395_at | NSF | 1.134 | 0.0242 |
| 202622_s_at | ATXN2 | 1.134 | 0.0346 |
| 208660_at | CS | 1.134 | 0.0376 |
| 209239_at | NFKB1 | 1.134 | 0.036 |
| 210685_s_at | UBE4B | 1.134 | 0.0376 |
| 212470_at | SPAG9 | 1.134 | 0.0498 |
| 213604_at | ELOA | 1.134 | 0.0149 |
| 218569_s_at | KBTBD4 | 1.134 | 0.0294 |
| 201713_s_at | RANBP2 | 1.133 | 0.024 |
| 212053_at | PDXDC1 | 1.133 | 0.0122 |
| 215792_s_at | DNAJC11 | 1.133 | 0.0372 |
| 215533_s_at | UBE4B | 1.132 | 0.0479 |
| 204245_s_at | RPP14 | 1.131 | 0.0466 |
| 201218_at | CTBP2 | 1.13 | 0.0397 |
| 203334_at | DHX8 | 1.13 | 0.0361 |
| 212163_at | KIDINS220 | 1.13 | 0.0494 |
| 212957_s_at | LINC01278 | 1.13 | 0.0149 |
| 217403_s_at | ZNF227 | 1.13 | 0.0433 |
| 212832_s_at | CKAP5 | 1.129 | 0.0234 |
| 200683_s_at | UBE2L3 | 1.128 | 0.0395 |
| 200854_at | NCOR1 | 1.128 | 0.0305 |
| 201968_s_at | PGM1 | 1.128 | 0.0426 |
| 202521_at | CTCF | 1.128 | 0.0097 |
| 206665_s_at | BCL2L1 | 1.128 | 0.0363 |
| 217076_s_at | HOXD3 | 1.128 | 0.0376 |
| 200765_x_at | CTNNA1 | 1.127 | 0.0091 |
| 201706_s_at | PEX19 | 1.127 | 0.028 |
| 203585_at | ZNF185 | 1.127 | 0.028 |
| 209139_s_at | PRKRA | 1.127 | 0.0288 |
| 211662_s_at | VDAC2 | 1.127 | 0.0188 |
| 200693_at | YWHAQ | 1.125 | 0.0072 |
| 208915_s_at | GGA2 | 1.125 | 0.0166 |
| 214647_s_at | HFE | 1.125 | 0.0296 |
| 217746_s_at | PDCD6IP | 1.125 | 0.0146 |
| 200643_at | HDLBP | 1.124 | 0.0146 |
| 204764_at | FNTB | 1.124 | 0.0376 |
| 202100_at | RALB | 1.123 | 0.0147 |
| 201985_at | WASHC5 | 1.122 | 0.0426 |
| 208905_at | CYCS | 1.122 | 0.0328 |
| 212429_s_at | GTF3C2 | 1.122 | 0.0398 |
| 201021_s_at | DSTN | 1.121 | 0.0278 |
| 202491_s_at | ELP1 | 1.121 | 0.0386 |
| 208617_s_at | PTP4A2 | 1.121 | 0.0213 |
| 200708_at | GOT2 | 1.12 | 0.0259 |
| 207922_s_at | MAEA | 1.12 | 0.0318 |
| 209350_s_at | GPS2 | 1.12 | 0.0303 |
| 217758_s_at | TM9SF3 | 1.12 | 0.0242 |
| 201771_at | SCAMP3 | 1.119 | 0.0146 |
| 210844_x_at | CTNNA1 | 1.119 | 0.0084 |
| 201322_at | ATP5F1B | 1.118 | 0.0149 |
| 213762_x_at | RBMX | 1.118 | 0.0346 |
| 208619_at | DDB1 | 1.117 | 0.0153 |
| 208717_at | OXA1L | 1.117 | 0.0174 |
| 201384_s_at | NBR1 | 1.115 | 0.0165 |
| 211240_x_at | CTNND1 | 1.115 | 0.0287 |
| 213860_x_at | CSNK1A1 | 1.115 | 0.0387 |
| 217448_s_at | TOX4 | 1.115 | 0.0382 |
| 217920_at | MAN1A2 | 1.115 | 0.0418 |
| 211558_s_at | DHPS | 1.114 | 0.034 |
| 201190_s_at | PITPNA | 1.113 | 0.0311 |
| 206576_s_at | CEACAM1 | 1.113 | 0.0446 |
| 213190_at | COG7 | 1.113 | 0.0446 |
| 201176_s_at | ARCN1 | 1.112 | 0.0097 |
| 212630_at | EXOC3 | 1.112 | 0.0446 |
| 217795_s_at | TMEM43 | 1.111 | 0.0346 |
| 200030_s_at | SLC25A3 | 1.11 | 0.0094 |
| 211427_s_at | KCNJ13 | 1.11 | 0.0431 |
| 200816_s_at | PAFAH1B1 | 1.109 | 0.0194 |
| 201175_at | TMX2 | 1.109 | 0.0409 |
| 208608_s_at | SNTB1 | 1.109 | 0.0315 |
| 212142_at | MCM4 | 1.109 | 0.0337 |
| 222021_x_at | SDHAP1 | 1.109 | 0.0385 |
| 203353_s_at | MBD1 | 1.108 | 0.017 |
| 204992_s_at | PFN2 | 1.108 | 0.0277 |
| 206621_s_at | EIF4H | 1.108 | 0.0372 |
| 201800_s_at | OSBP | 1.107 | 0.0275 |
| 203267_s_at | DRG2 | 1.107 | 0.0337 |
| 200855_at | NCOR1 | 1.106 | 0.0433 |
| 211078_s_at | STK3 | 1.106 | 0.0387 |
| 200065_s_at | ARF1 | 1.105 | 0.0496 |
| 201390_s_at | CSNK2B | 1.105 | 0.043 |
| 202585_s_at | NFX1 | 1.105 | 0.0188 |
| 214991_s_at | PIGO | 1.105 | 0.0346 |
| 208778_s_at | TCP1 | 1.104 | 0.0363 |
| 201191_at | PITPNA | 1.102 | 0.0415 |
| 201969_at | NASP | 1.102 | 0.0453 |
| 207279_s_at | NEBL | 1.101 | 0.0496 |
| 212072_s_at | CSNK2A1 | 1.101 | 0.0146 |
| 202738_s_at | PHKB | 1.099 | 0.0341 |
| 203082_at | BMS1 | 1.099 | 0.0397 |
| 221486_at | ENSA | 1.098 | 0.0426 |
| 200595_s_at | EIF3A | 1.097 | 0.0346 |
| 200794_x_at | DAZAP2 | 1.096 | 0.0147 |
| 212696_s_at | RNF4 | 1.096 | 0.0347 |
| 213738_s_at | ATP5F1A | 1.096 | 0.036 |
| 200886_s_at | PGAM1 | 1.094 | 0.0072 |
| 217595_at | GSPT1 | 1.093 | 0.0376 |
| 220607_x_at | NELFCD | 1.091 | 0.0346 |
| 200668_s_at | UBE2D3 | 1.09 | 0.0431 |
| 217839_at | TFG | 1.09 | 0.017 |
| 202802_at | DHPS | 1.089 | 0.0313 |
| 200860_s_at | CNOT1 | 1.088 | 0.0372 |
| 221767_x_at | HDLBP | 1.086 | 0.0288 |
| 208724_s_at | RAB1A | 1.084 | 0.0134 |
| 207125_at | ZNF225 | 1.079 | 0.0364 |
| 209128_s_at | SART3 | 1.076 | 0.0331 |
| 200614_at | CLTC | 1.073 | 0.0273 |
| 209174_s_at | QRICH1 | 1.073 | 0.034 |
| 214585_s_at | VPS52 | 1.071 | 0.0382 |
| 209517_s_at | ASH2L | 1.07 | 0.0346 |
| 201442_s_at | ATP6AP2 | 1.065 | 0.0379 |
| 200004_at | EIF4G2 | 1.064 | 0.0466 |
| 220799_at | GCM2 | −1.03 | 0.0327 |
| 215831_at | AF113018 | −1.041 | 0.0275 |
| 221696_s_at | STYK1 | −1.044 | 0.028 |
| 215018_at | CEP295 | −1.047 | 0.0478 |
| 210258_at | RGS13 | −1.048 | 0.0215 |
| 207496_at | MS4A2 | −1.053 | 0.0296 |
| 214405_at | AF035318 | −1.053 | 0.0346 |
| 200095_x_at | RPS10 | −1.056 | 0.0147 |
| 208280_at | CDRT1 | −1.061 | 0.0265 |
| 205493_s_at | DPYSL4 | −1.065 | 0.0331 |
| 204269_at | PIM2 | −1.072 | 0.0497 |
| 210634_at | KLHL20 | −1.073 | 0.0365 |
| 207988_s_at | ARPC2 | −1.076 | 0.0476 |
| 206131_at | CLPS | −1.077 | 0.0365 |
| 207194_s_at | ICAM4 | −1.079 | 0.0428 |
| 216589_at | N/A | −1.081 | 0.0494 |
| 205721_at | GFRA2 | −1.083 | 0.0154 |
| 203538_at | CAMLG | −1.084 | 0.0489 |
| 61874_at | CACFD1 | −1.085 | 0.0339 |
| 208587_s_at | OR1E1 | −1.087 | 0.0494 |
| 206680_at | CD5L | −1.089 | 0.0346 |
| 208054_at | HERC4 | −1.09 | 0.0275 |
| 216606_x_at | LYPLA2 | −1.096 | 0.0494 |
| 221113_s_at | WNT16 | −1.096 | 0.0275 |
| 204697_s_at | CHGA | −1.098 | 0.0382 |
| 205055_at | ITGAE | −1.099 | 0.0397 |
| 219594_at | NINJ2 | −1.101 | 0.0304 |
| 210222_s_at | RTN1 | −1.102 | 0.0363 |
| 215536_at | HLA-DQB2 | −1.105 | 0.0179 |
| 216957_at | USP22 | −1.105 | 0.0084 |
| 219019_at | PIDD1 | −1.105 | 0.0188 |
| 216573_at | IGLV1-40 | −1.109 | 0.0304 |
| 209686_at | S100B | −1.111 | 0.0446 |
| 204198_s_at | RUNX3 | −1.112 | 0.0346 |
| 217145_at | AF103574 | −1.113 | 0.0107 |
| 213958_at | CD6 | −1.114 | 0.0107 |
| 222001_x_at | N/A | −1.114 | 0.0129 |
| 215568_x_at | LYPLA2 | −1.115 | 0.022 |
| 216789_at | TMEM92-AS1 | −1.116 | 0.0381 |
| 220575_at | FAM106A | −1.116 | 0.0331 |
| 202305_s_at | FEZ2 | −1.117 | 0.0444 |
| 209166_s_at | MAN2B1 | −1.118 | 0.0286 |
| 213527_s_at | ZNF688 | −1.118 | 0.0324 |
| 210321_at | GZMH | −1.119 | 0.0469 |
| 220024_s_at | PRX | −1.119 | 0.0498 |
| 222211_x_at | SCAND2P | −1.119 | 0.0097 |
| 64064_at | GIMAP5 | −1.119 | 0.0382 |
| 221462_x_at | KLK15 | −1.12 | 0.005 |
| 209235_at | CLCN7 | −1.121 | 0.027 |
| 202191_s_at | GAS7 | −1.122 | 0.0288 |
| 212886_at | CCDC69 | −1.122 | 0.0346 |
| 202040_s_at | KDM5A | −1.123 | 0.0396 |
| 215621_s_at | IGHD | =1.123 | 0.017 |
| 218913_s_at | GMIP | −1.124 | 0.0086 |
| 209879_at | SELPLG | −1.125 | 0.0287 |
| 217312_s_at | COL7A1 | −1.125 | 0.0384 |
| 218346_s_at | SESN1 | −1.125 | 0.0082 |
| 204077_x_at | ENTPD4 | −1.126 | 0.0363 |
| 204336_s_at | RGS19 | −1.126 | 0.0376 |
| 203675_at | NUCB2 | −1.128 | 0.0382 |
| 206121_at | AMPD1 | −1.129 | 0.0097 |
| 217360_x_at | IGHJ3 | −1.129 | 0.0324 |
| 217764_s_at | RAB31 | −1.13 | 0.0417 |
| 210448_s_at | P2RX5 | −1.132 | 0.0231 |
| 216558_x_at | IGHG3 | −1.133 | 0.0321 |
| 217198_x_at | IGHJ3 | −1.133 | 0.0226 |
| 207133_x_at | ALPK1 | −1.136 | 0.0376 |
| 210629_x_at | LST1 | −1.136 | 0.029 |
| 212873_at | ARHGAP45 | −1.136 | 0.005 |
| 38149_at | ARHGAP25 | −1.136 | 0.0327 |
| 201723_s_at | GALNT1 | −1.137 | 0.024 |
| 209924_at | CCL18 | −1.138 | 0.0296 |
| 205641_s_at | TRADD | −1.139 | 0.0126 |
| 221710_x_at | EVA1B | −1.14 | 0.0208 |
| 209534_x_at | AKAP13 | −1.142 | 0.0275 |
| 205639_at | AOAH | −1.144 | 0.0213 |
| 219468_s_at | CUEDC1 | −1.144 | 0.0371 |
| 214656_x_at | MYO1C | −1.148 | 0.0444 |
| 205180_s_at | ADAM8 | −1.149 | 0.0252 |
| 221978_at | HLA-F | −1.149 | 0.0088 |
| 204923_at | SASH3 | −1.15 | 0.0296 |
| 207741_x_at | TPSAB1 | −1.152 | 0.0425 |
| 210769_at | CNGB1 | −1.153 | 0.0285 |
| 209225_x_at | TNPO1 | −1.154 | 0.0278 |
| 204786_s_at | IFNAR2 | −1.155 | 0.0494 |
| 202947_s_at | GYPC | −1.156 | 0.015 |
| 216829_at | IGKV1-17 | −1.157 | 0.0186 |
| 204948_s_at | FST | −1.161 | 0.0351 |
| 217549_at | NCKAP1L | −1.161 | 0.0304 |
| 216542_x_at | IGHV3-20 | −1.162 | 0.0464 |
| 33304_at | ISG20 | −1.164 | 0.044 |
| 211635_x_at | IGHV1-69 | −1.165 | 0.0331 |
| 219183_s_at | CYTH4 | −1.166 | 0.0088 |
| 215633_x_at | LST1 | −1.168 | 0.0147 |
| 204882_at | ARHGAP25 | −1.169 | 0.0097 |
| 211192_s_at | CD84 | −1.169 | 0.0149 |
| 202096_s_at | TSPO | −1.172 | 0.0364 |
| 213193_x_at | TRBV19 | −1.172 | 0.048 |
| 209579_s_at | MBD4 | −1.175 | 0.0201 |
| 211908_x_at | IGK | −1.175 | 0.0421 |
| 204319_s_at | RGS10 | −1.176 | 0.0185 |
| 211633_x_at | IGHG1 | −1.178 | 0.028 |
| 209083_at | CORO1A | −1.18 | 0.0491 |
| 214574_x_at | LST1 | −1.18 | 0.0128 |
| 202156_s_at | CELF2 | −1.181 | 0.0084 |
| 204365_s_at | REEP1 | −1.185 | 0.0385 |
| 219117_s_at | FKBP11 | −1.191 | 0.0476 |
| 209906_at | C3AR1 | −1.192 | 0.031 |
| 205098_at | CCR1 | −1.193 | 0.0376 |
| 216412_x_at | IGLV1-40 | −1.193 | 0.0064 |
| 213160_at | DOCK2 | −1.196 | 0.0061 |
| 217763_s_at | RAB31 | −1.2 | 0.0187 |
| 210084_x_at | TPSAB1 | −1.201 | 0.0208 |
| 203507_at | CD68 | −1.203 | 0.0107 |
| 214181_x_at | LST1 | −1.203 | 0.0156 |
| 221698_s_at | CLEC7A | −1.204 | 0.0242 |
| 204220_at | GMFG | −1.206 | 0.0086 |
| 211650_x_at | IGK | −1.207 | 0.0185 |
| 204912_at | IL10RA | −1.208 | 0.0151 |
| 204057_at | IRF8 | −1.209 | 0.0408 |
| 214470_at | KLRB1 | −1.209 | 0.039 |
| 205831_at | CD2 | −1.211 | 0.0208 |
| 209354_at | TNFRSF14 | −1.211 | 0.0078 |
| 202435_s_at | CYP1B1 | −1.212 | 0.0461 |
| 212119_at | RHOQ | −1.212 | 0.0088 |
| 214916_x_at | IGHV3-23 | −1.213 | 0.0426 |
| 221666_s_at | PYCARD | −1.216 | 0.0208 |
| 38487_at | STAB1 | −1.218 | 0.0054 |
| 204150_at | STAB1 | −1.219 | 0.0129 |
| 205683_x_at | TPSAB1 | −1.221 | 0.0165 |
| 211639_x_at | SKAP2 | −1.223 | 0.0304 |
| 207134_x_at | TPSB2 | −1.227 | 0.0107 |
| 202957_at | HCLS1 | −1.231 | 0.0395 |
| 217281_x_at | IGHG1 | −1.231 | 0.0473 |
| 204971_at | CSTA | −1.234 | 0.0363 |
| 214770_at | MSR1 | −1.234 | 0.0104 |
| 218960_at | TMPRSS4 | −1.235 | 0.0042 |
| 209606_at | CYTIP | −1.239 | 0.0442 |
| 204232_at | FCER1G | −1.241 | 0.0494 |
| 216365_x_at | IGLJ3 | −1.245 | 0.0242 |
| 1405_i_at | CCL5 | −1.246 | 0.0446 |
| 205952_at | KCNK3 | −1.247 | 0.0083 |
| 203760_s_at | SLA | −1.249 | 0.0082 |
| 211634_x_at | IGHG1 | −1.254 | 0.0138 |
| 206208_at | CA4 | −1.255 | 0.0149 |
| 202800_at | SLC1A3 | −1.256 | 0.0146 |
| 216510_x_at | IGHV3-23 | −1.267 | 0.0442 |
| 204118_at | CD48 | −1.268 | 0.0072 |
| 206209_s_at | CA4 | −1.271 | 0.0449 |
| 204446_s_at | ALOX5 | −1.273 | 0.0186 |
| 213813_x_at | AI345238 | −1.275 | 0.0346 |
| 204829_s_at | FOLR2 | −1.276 | 0.0024 |
| 210915_x_at | TRBC1 | −1.276 | 0.0105 |
| 211881_x_at | IGLJ3 | −1.278 | 0.0175 |
| 221286_s_at | MZB1 | −1.278 | 0.005 |
| 201721_s_at | LAPTM5 | −1.282 | 0.0242 |
| 222303_at | ETS2 | −1.287 | 0.0457 |
| 213539_at | CD3D | −1.288 | 0.0037 |
| 213566_at | RNASE6 | =1.29 | 0.0233 |
| 211868_x_at | IGLJ3 | −1.295 | 0.0185 |
| 205419_at | GPR183 | −1.299 | 0.0149 |
| 213674_x_at | IGHD | −1.303 | 0.0086 |
| 217227_x_at | N/A | −1.303 | 0.0042 |
| 215214_at | IGLV3-25 | −1.304 | 0.0187 |
| 216491_x_at | IGHM | −1.304 | 0.0169 |
| 211637_x_at | IGHV4-59 | −1.306 | 0.0147 |
| 204655_at | CCL5 | −1.31 | 0.0165 |
| 204787_at | VSIG4 | −1.311 | 0.0411 |
| 215388_s_at | CFH | −1.311 | 0.0426 |
| 216560_x_at | IGLV3-10 | −1.311 | 0.024 |
| 219666_at | MS4A6A | −1.317 | 0.0157 |
| 217258_x_at | IGLV1-40 | −1.319 | 0.0056 |
| 205267_at | POU2AF1 | −1.322 | 0.0084 |
| 203305_at | F13A1 | −1.325 | 0.0181 |
| 205798_at | IL7R | −1.343 | 0.0223 |
| 217480_x_at | N/A | −1.347 | 0.0147 |
| 34210_at | CD52 | −1.358 | 0.0479 |
| 217235_x_at | IGLV@ | −1.361 | 0.009 |
| 205624_at | CPA3 | −1.363 | 0.0305 |
| 212587_s_at | PTPRC | −1.365 | 0.0282 |
| 214149_s_at | ATP6V0E1 | −1.367 | 0.0114 |
| 209173_at | AGR2 | −1.373 | 0.0232 |
| 204259_at | MMP7 | −1.376 | 0.0195 |
| 209795_at | CD69 | −1.377 | 0.0048 |
| 211654_x_at | HLA-DQB1 | −1.387 | 0.017 |
| 204122_at | TYROBP | −1.392 | 0.005 |
| 204774_at | EVI2A | −1.395 | 0.0078 |
| 211643_x_at | IGKC | −1.396 | 0.0294 |
| 217157_x_at | IGKC | −1.398 | 0.0107 |
| 214777_at | IGKV4-1 | −1.403 | 0.03 |
| 216853_x_at | IGLV3-19 | −1.409 | 0.005 |
| 218232_at | C1QA | −1.411 | 0.0146 |
| 204661_at | CD52 | −1.412 | 0.0072 |
| 217179_x_at | IGLV1-51 | −1.416 | 0.0037 |
| 211798_x_at | IGLJ3 | −1.417 | 0.0131 |
| 219607_s_at | MS4A4A | −1.423 | 0.0215 |
| 202953_at | C1QB | −1.441 | 0.0072 |
| 216984_x_at | IGLJ3 | −1.447 | 0.0156 |
| 205433_at | BCHE | −1.456 | 0.0231 |
| 40665_at | FMO3 | −1.462 | 0.0471 |
| 217028_at | CXCR4 | −1.471 | 0.0082 |
| 217148_x_at | IGLV2-14 | −1.483 | 0.0149 |
| 213502_x_at | GUSBP11 | −1.498 | 0.0053 |
| 214768_x_at | IGKV2D-28 | −1.498 | 0.0091 |
| 213975_s_at | LYZ | −1.546 | 0.0189 |
| 216207_x_at | IGKV1D-13 | −1.548 | 0.0146 |
| 214836_x_at | IGKC | −1.56 | 0.0201 |
| 217378_x_at | IGKV1OR2-108 | −1.583 | 0.0107 |
| 212999_x_at | HLA-DQB1 | −1.584 | 0.0042 |
| 202238_s_at | NNMT | −1.594 | 0.0346 |
| 216401_x_at | IGKV1-37 | −1.611 | 0.0089 |
| 216576_x_at | IGKC | −1.636 | 0.0156 |
| 202237_at | NNMT | −1.638 | 0.0214 |
| 210072_at | CCL19 | −1.683 | 0.0267 |
| 214669_x_at | IGKC | −1.687 | 0.0486 |
| 215946_x_at | IGLL3P | −1.691 | 0.0042 |
| 211644_x_at | IGKC | −1.713 | 0.0088 |
| 211645_x_at | IGKV1-17 | −1.75 | 0.0086 |
| 215379_x_at | IGLL5 | −1.809 | 0.0261 |
| 215121_x_at | IGLL5 | −1.832 | 0.0379 |
| 215176_x_at | IGKC | −1.861 | 0.0208 |
| 209138_x_at | IGLL5 | −1.886 | 0.0357 |
| 209480_at | HLA-DQB1 | −1.907 | 0.0107 |
| 214677_x_at | IGLL5 | −1.915 | 0.0488 |
| 212592_at | JCHAIN | −1.944 | 0.0289 |
| 213831_at | HLA-DQA1 | −2.038 | 0.0061 |
| 211430_s_at | IGHM | −2.097 | 0.0319 |

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Hart A, Lentine K L, Smith J M, et al. OPTN/SRTR 2019 Annual Data Report: Kidney. Am J Transplant. 2021; 21 Suppl 2:21-137. doi:10.1111/ajt.16502
2. Ojo A O. Expanded criteria donors: process and outcomes. Semin Dial. 2005; 18(6):463-468. doi:10.1111/j.1525-139X.2005.00090.x
3. Filiopoulos V, Boletis J N. Renal transplantation with expanded criteria donors: Which is the optimal immunosuppression? World J Transplant. 2016; 6(1):103-114. doi:10.5500/wjt.v6.i1.103
4. Rao P S, Ojo A. The alphabet soup of kidney transplantation: SCD, DCD, ECD-fundamentals for the practicing nephrologist. Clin J Am Soc Nephrol. 2009; 4(11):1827-1831. doi:10.2215/CJN.02270409
5. Dahmen M, Becker F, Pavenstadt H, Suwelack B, Schatte-Natgen K, Reuter S. Validation of the Kidney Donor Profile Index (KDPI) to assess a deceased donor's kidneys' outcome in a European cohort. Sci Rep. 2019; 9(1):11234. doi:10.1038/s41598-019-47772-7
6. Lentine K L, Kasiske B, Axelrod D A. Procurement Biopsies in Kidney Transplantation: More Information May Not Lead to Better Decisions. J Am Soc Nephrol. 2021; 32(8):1835-1837. doi:10.1681/ASN.2021030403
7. Gaber L W, Moore L W, Alloway R R, Amiri M H, Vera S R, Gaber A O. Glomerulosclerosis as a determinant of posttransplant function of older donor renal allografts. Transplantation. 1995; 60(4):334-339. doi: 10,1097/00007890-199508270-00006
8. von Moos S, Akalin E, Mas V, Mueller T F. Assessment of Organ Quality in Kidney Transplantation by Molecular Analysis and Why It May Not Have Been Achieved, Yet. Front Immunol. 2020; 11:833. doi: 10.3389/fimmu.2020.00833
9. Supplitt S, Karpinski P, Sasiadek M, Laczmanska I. Current Achievements and Applications of Transcriptomics in Personalized Cancer Medicine. Int J Mol Sci. 2021; 22(3):1422. doi:10.3390/ijms22031422
10. Supplitt S, Karpinski P, Sasiadek M, Laczmanska I. Current Achievements and Applications of Transcriptomics in Personalized Cancer Medicine. Int J Mol Sci. 2021; 22(3):1422. Published Jan. 31, 2021. doi:10.3390/ijms22031422
11. Irizarry R A, Hobbs B, Collin F, et al. Exploration, normalization, and summaries of high density oligonucleotide array probe level data. Biostatistics. 2003; 4(2):249-264. doi:10.1093/biostatistics/4.2.249
12. Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann Intern Med. 1999; 130(6):461-470. doi:10.7326/0003-4819-130-6-199903160-00002
13. Benjamini Y, Hochberg Y. Controlling the False Discovery Rate: A Practical and Powerful Approach to Multiple Testing. J R Stat Soc Ser B Methodol. 1995; 57(1):289-300. doi:10.1111/j.2517-6161.1995.tb02031.x
14. Kuhn M. Building Predictive Models in R Using the caret Package. J Stat Softw. 2008; 28(1):1-26. doi:10.18637/jss.v028.i05
15. Friedman J, Hastie T, Tibshirani R. Regularization Paths for Generalized Linear Models via Coordinate Descent. J Stat Softw. 2010; 33(1):1-22.
16. Harrell F E. Multivariable Modeling Strategies. In: Harrell Jr Frank E, ed. Regression Modeling Strategies: With Applications to Linear Models, Logistic and Ordinal Regression, and Survival Analysis. Springer Series in Statistics. Springer International Publishing; 2015:63-102. doi:10.1007/978-3-319-19425-7_4
17. Zhou Y, Zhou B, Pache L, et al. Metascape provides a biologist-oriented resource for the analysis of systems-level datasets. Nat Commun. 2019; 10(1):1523. Published Apr. 3, 2019. doi:10.1038/s41467-019-09234-6
18. Herath S, Dai H, Erlich J, et al. Selection and validation of reference genes for normalisation of gene expression in ischaemic and toxicological studies in kidney disease. PLoS One. 2020; 15(5): e0233109. doi:10.1371/journal.pone.0233109
19. Reese P P, Harhay M N, Abt P L, Levine M H, Halpern S D. New Solutions to Reduce Discard of Kidneys Donated for Transplantation. J Am Soc Nephrol. 2016; 27(4):973-980. doi:10.1681/ASN.2015010023
20. Moeckli B, Sun P, Lazeyras F, et al. Evaluation of donor kidneys prior to transplantation: an update of current and emerging methods. Transpl Int. 2019; 32(5):459-469. doi:10.1111/tri.13430
21. Stegall M D, Gaston R S, Cosio F G, Matas A. Through a glass darkly: seeking clarity in preventing late kidney transplant failure. J Am Soc Nephrol. 2015; 26(1):20-29. doi:10.1681/ASN.2014040378
22. Bae S, Massie A B, Thomas A G, et al. Who can tolerate a marginal kidney? Predicting survival after deceased donor kidney transplant by donor-recipient combination. Am J Transplant. 2019; 19(2):425-433. doi:10.1111/ajt.14978
23. Husain S A, Shah V, Alvarado Verduzco H, et al. Impact of Deceased Donor Kidney Procurement Biopsy Technique on Histologic Accuracy. Kidney Int Rep. 2020:5(11):1906-1913. Published Aug. 14, 2020. doi:10.1016/j.ekir.2020.08.004
24. Hall I E, Parikh C R, Schröppel B, et al. Procurement Biopsy Findings Versus Kidney Donor Risk Index for Predicting Renal Allograft Survival. Transplant Direct. 2018; 4(8): e373. doi:10.1097/TXD.0000000000000816
25. Zhong Y, Schaubel D E, Kalbfleisch J D, Ashby V B, Rao P S, Sung R S. Reevaluation of the Kidney Donor Risk Index. Transplantation. 2019; 103(8):1714-1721 doi: 10.1097/TP.0000000000002498
26. Hauser P, Schwarz C, Mitterbauer C, et al. Genome-wide gene-expression patterns of donor kidney biopsies distinguish primary allograft function. Lab Invest. 2004; 84(3):353-361. doi:10.1038/labinvest.3700037
27. Kainz A, Perco P, Mayer B, et al. Gene-expression profiles and age of donor kidney biopsies obtained before transplantation distinguish medium term graft function. Transplantation. 2007; 83(8):1048-1054. doi:10.1097/01.tp.0000259960.56786.ec
28. Mueller T F, Reeve J, Jhangri G S, et al. The transcriptome of the implant biopsy identifies donor kidneys at increased risk of delayed graft function. Am J Transplant. 2008; 8(1):78-85. doi:10.1111/j.1600-6143.2007.02032.x
29. Mas V R, Archer K J, Yanek K. et al. Gene expression patterns in deceased donor kidneys developing delayed graft function after kidney transplantation. Transplantation. 2008; 85(4):626-635. doi:10.1097/TP.0b013e318165491f
30. Bodonvi-Kovacs G. Putheti P, Marino M, et al. Gene expression profiling of the donor kidney at the time of transplantation predicts clinical outcomes 2 years after transplantation. Hum Immunol. 2010; 71(5):451-455. doi:10.1016/j.humimm.2010.02.013
31. Mas V R, Scian M J, Archer K J, et al. Pretransplant transcriptome profiles identify among kidneys with delayed graft function those with poorer quality and outcome. Mol Med. 2011; 17(11-12):1311-1322. doi:10.2119/molmed.2011.00159
32. Goncalves-Primo A, Mourão TB, Andrade-Oliveira V, et al. Investigation of apoptosis-related gene expression levels in preimplantation biopsies as predictors of delayed kidney graft function. Transplantation. 2014; 97(12):1260-1265. doi:10.1097/01.TP.0000442579.12285.e8
33. McGuinness D, Mohammed S, Monaghan L, et al. A molecular signature for delayed graft function. Aging Cell. 2018; 17(5): e12825. doi:10.111/acel.12825
34. Yang J, Snijders M L H, Haasnoot G W, et al. Elevated intragraft expression of innate immunity and cell death-related markers is a risk factor for adverse graft outcome. Transpl Immunol. 2018; 48:39-46. doi:10.1016/j.trim.2018.02.009
35. Guerrieri D, Re L, Petroni J, et al. Gene expression profile in delay graft function: inflammatory markers are associated with recipient and donor risk factors. Mediators Inflamm. 2014; 2014:167361. doi:10.1155/2014/167361
36. Ferdinand J R, Hosgood S A, Moore T, et al. Cytokine absorption during human kidney perfusion reduces delayed graft function-associated inflammatory gene signature. Am J Transplant. 2021; 21(6):2188-2199. doi:10.1111/ajt.16371
37. Mueller T F, Solez K, Mas V. Assessment of kidney organ quality and prediction of outcome at time of transplantation. Semin Immunopathol. 2011; 33(2):185-199. doi: 10.1007/s00281-011-0248-x
38. Cippá PE, Liu J, Sun B, Kumar S, Naesens M, McMahon A P. A late B lymphocyte action in dysfunctional tissue repair following kidney injury and transplantation. Nat Commun. 2019; 10(1):1157. Published Mar. 11, 2019. doi:10.1038/s41467-019-09092-2
39. Kreepala C, Famulski K S, Chang J, Halloran P F. Comparing molecular assessment of implantation biopsies with histologic and demographic risk assessment. Am J Transplant. 2013; 13(2):415-426. doi:10.1111/ajt.12043
40. Kaminska D, Kościelska-Kasprzak K, Mazanowska 0, et al. Pretransplant Immune Interplay Between Donor and Recipient Influences Posttransplant Kidney Allograft Function. Transplant Proc. 2018; 50(6):1658-1661. doi: 10.1016/j.transproceed.2018.03.129
41. Lim M A, Bloom R D. Medical Therapies to Reduce Delayed Graft Function and Improve Long-Term Graft Survival: Are We Making Progress?. Clin J Am Soc Nephrol. 2020; 15(1):13-15 doi:10.2215/CJN.13961119
42. Kayler L K, Sokolich J, Magliocca J, Schold J D. Import kidney transplants from nonmandatory share deceased donors: characteristics, distribution and outcomes. J Transplant. 2011; 11(1):77-85. doi:10.111l/j.1600-6143.2010.03359.x
43. Le Meur Y, Aulagnon F, Bertrand D, et al. Effect of an Early Switch to Belatacept Among Calcineurin Inhibitor-Intolerant Graft Recipients of Kidneys From Extended-Criteria Donors. Am J Transplant. 2016; 16(7):2181-2186. doi:10.1111/ajt.13698
44. Scian M J, Maluf D G, Archer K J, et al. Identification of biomarkers to assess organ quality and predict posttransplantation outcomes. Transplantation. 2012; 94(8):851-858. doi:10.1097/TP.0b013e318263702b
45. Bontha S V, Maluf D G, Archer K J, et al. Effects of DNA Methylation on Progression to Interstitial Fibrosis and Tubular Atrophy in Renal Allograft Biopsies: A Multi-Omics Approach. Am J Transplant. 2017; 17(12):3060-3075. doi:10.1111/ajt.14372
46. Kingsmore K M, Bachali P, Catalina M D, et al. Altered expression of genes controlling metabolism characterizes the tissue response to immune injury in lupus. Sci Rep. 2021; 11(1):14789. Published Jul. 20, 2021. doi:10.1038/s41598-021-93034-w
47. Afshinnia F, Rajendiran T M, Soni T, et al. Impaired β-Oxidation and Altered Complex Lipid Fatty Acid Partitioning with Advancing CKD. J Am Soc Nephrol. 2018; 29(1):295-306. doi:10.1681/ASN.2017030350
48. Hallan S, Afkarian M, Zelnick L R, et al. Metabolomics and Gene Expression Analysis Reveal Down-regulation of the Citric Acid (TCA) Cycle in Non-diabetic CKD Patients. EBioMedicine. 2017; 26:68-77. doi:10.1016/j.ebiom.2017.10.027
49. Sharma K, Karl B, Mathew A V, et al. Metabolomics reveals signature of mitochondrial dysfunction in diabetic kidney disease. J Am Soc Nephrol. 2013; 24(11):1901-1912. doi:10.1681/ASN.2013020126
50. Grayson P C, Eddy S, Taroni J N, et al. Metabolic pathways and immunometabolism in rare kidney diseases. Ann Rheum Dis. 2018; 77(8): 1226-1233. doi:10.1136/annrheumdis-2017-212935
51. He J C, Chuang P Y, Ma'ayan A, Iyengar R. Systems biology of kidney diseases. Kidney Int. 2012; 81(1):22-39. doi:10.1038/ki.2011.314
52. Maggiore U, Leventhal J, Cravedi P. Rethinking clinical endpoints in kidney transplant trials Curr Opin Organ Transplant. 2020; 25(1):1-7. doi:10.1097/MOT.0000000000000719
53. Baek C H, Kim H, Yang W S. Han D J, Park S K. A postoperative 1-Year eGFR of More Than 45 ml/min May be the Cutoff Level for a Favorable Long-Term Prognosis in Renal Transplant Patients. Ann Transplant. 2016; 21:439-447. Published Jul. 15, 2016. doi:10.12659/aot.897938

1. A method of determining a graft function risk score for a kidney, comprising:
(a) obtaining a tissue sample from a kidney,
(b) measuring expression levels of one or more predictive genes in said sample,
(c) measuring expression levels of one or more housekeeping genes in said sample,
(d) calculating differences in expression levels measured for the one or more predictive genes versus expression levels measured for the one or more housekeeping genes, and
(e) calculating a graft function risk score based on the differences calculated in (d), wherein the graft function risk score is calculated using the following formula (I)
$$graft function risk score = {b}_{0} + {b}_{1} ( {X}_{1} ) + {b}_{2} ( {X}_{2} ) + … {b}_{p} ( {X}_{p} ) \qquad ( I )$$
wherein b₀ is the intercept in a logistic regression model, wherein each b₁₋ₚ is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample.

2. A method of determining a graft function risk score for a kidney, comprising:
(a) obtaining a tissue sample from a kidney,
(b) measuring expression levels of 13 predictive genes in said sample,
(c) measuring expression levels of two housekeeping genes in said sample,
(d) calculating differences in expression levels measured for each of the 13 predictive genes versus the mean value of expression levels measured for the two housekeeping genes, and
(e) calculating a graft function risk score based on the differences calculated in (d), wherein the graft function risk score is calculated using the following formula (II)
$$graft function risk score = - 4.544 + 0.29 ( Δ Ct BCHE ) + 0.023 ( Δ Ct FKBP 4 ) - 0.981 ( Δ Ct GYPC ) - 0.105 ( Δ Ct HLA - DQB 1 ) - 0.327 ( Δ Ct HNRNPH 3 ) + 0.039 ( Δ Ct IGHD ) + 0.975 ( Δ Ct NUDT 4 ) + 0.717 ( Δ Ct RBM 8 A ) - 2.182 ( Δ Ct RHOQ ) + 0.112 ( Δ Ct SQLE ) + 1.073 ( Δ Ct STK 24 ) + 0.171 ( Δ Ct TRADD ) + 0.378 ( Δ Ct ZNFI 85 ) + 0.057 ( donor age ) + 0.004 ( donor BMI ) + 0.586 ( donor race indicator variable ) \qquad ( II )$$
wherein the donor race indicator variable=0 for Caucasian and 1 for all other races,
wherein the 13 predictive genes are BCHE, FKBP4, GYPC, HLA-DQB1, HNRNPH3, IGHD, NUDT4, RBM8A, RHOQ, SQLE, STK24, TRADD, and ZNF185,
wherein the two housekeeping genes are ACTB and GAPDH, and
wherein each ΔCt in formula (II) represents the calculated difference in expression level of indicated predictive genes versus the mean value of expression levels for the two housekeeping genes for each of the 13 predictive genes.

3. The method of claim 1, further comprising converting the risk score into a probability score for a 0.0-1.0 probability scale, wherein the probability score is calculated using the following formula (III)
$$Probability score = \frac{{e}^{( {b}_{0} + {b}_{1} {X}_{1} + {b}_{2} {X}_{2} + ⋯ {b}_{p} {X}_{p} )}}{1 + {e}^{( {b}_{0} + {b}_{1} {X}_{1} + {b}_{2} {X}_{2} + ⋯ {b}_{p} {X}_{p} )}} \qquad ( III )$$
wherein b₀ is the intercept in a logistic regression model, wherein each b₁₋ₚ is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample, and e=2.71828.

4. The method of claim 1, wherein the predictive genes are selected from the group consisting of BCHE, FKBP4, GYPC, HLA-DQB1, HNRNPH3, IGHD, NUDT4, RBM8A, RHOQ, SQLE, STK24, TRADD, and ZNF185.

5. The method of claim 1, wherein the housekeeping genes are selected from the group consisting of ACTB and GAPDH.

6. The method of claim 1, wherein the kidney is a donor kidney.

7. The method of claim 1, wherein the expression levels of the genes are measured using qPCR.

8. The method of claim 1, wherein the calculated differences in expression levels measured for the one or more predictive genes versus expression levels measured for the one or more housekeeping genes in (d), is calculated using the mean value of expression levels measured for the housekeeping genes when the expression levels of two or more housekeeping genes are measured.

9. The method of claim 1, wherein the graft function risk score is one consideration in a decision of whether to transplant the kidney into a transplant recipient.

10. The method of claim 3, wherein the probability score is one consideration in a decision of whether to transplant the kidney into a transplant recipient.

11. The method of claim 1, wherein the graft function risk score is used to predict whether the kidney will continue to function for at least 24 months in a transplant recipient receiving the kidney.

12. The method of claim 3, wherein the probability score is the probability that the kidney will continue to function for at least 24 months in a transplant recipient receiving the kidney.

13. The method of claim 2, further comprising converting the risk score into a probability score for a 0.0-1.0 probability scale, wherein the probability score is calculated using the following formula (III)
$$Probability score = \frac{{e}^{( {b}_{0} + {b}_{1} {X}_{1} + {b}_{2} {X}_{2} + ⋯ {b}_{p} {X}_{p} )}}{1 + {e}^{( {b}_{0} + {b}_{1} {X}_{1} + {b}_{2} {X}_{2} + ⋯ {b}_{p} {X}_{p} )}} \qquad ( III )$$
wherein b₀ is the intercept in a logistic regression model, wherein each b₁₋ₚ is a regression coefficient for each independent value X₁₋ₚ, and wherein each X₁₋ₚ is the calculated difference in expression level measured for one predictive gene versus expression level measured for one or more housekeeping genes, and p is the number of predictive genes assayed in the tissue sample, and e=2.71828.

14. The method of claim 2, wherein the predictive genes are selected from the group consisting of BCHE, FKBP4, GYPC, HLA-DQB1, HNRNPH3, IGHD, NUDT4, RBM8A, RHOQ, SQLE, STK24, TRADD, and ZNF185.

15. The method of claim 2, wherein the housekeeping genes are selected from the group consisting of ACTB and GAPDH.

16. The method of claim 2, wherein the calculated differences in expression levels measured for the one or more predictive genes versus expression levels measured for the one or more housekeeping genes in (d), is calculated using the mean value of expression levels measured for the housekeeping genes when the expression levels of two or more housekeeping genes are measured.

17. The method of claim 2, wherein the graft function risk score is one consideration in a decision of whether to transplant the kidney into a transplant recipient.

18. The method of claim 13, wherein the probability score is one consideration in a decision of whether to transplant the kidney into a transplant recipient.

19. The method of claim 2, wherein the graft function risk score is used to predict whether the kidney will continue to function for at least 24 months in a transplant recipient receiving the kidney.

20. The method of claim 13, wherein the probability score is the probability that the kidney will continue to function for at least 24 months in a transplant recipient receiving the kidney.
